(19) United States
(12) Patent Application Publication
(10) Pub. No.: US 2026/0218297 A1
(43) Pub. Date: Jul. 30, 2026

(54) SEQUENCING PRIMER AND SEQUENCING METHOD

(71) Applicants: GENEMIND BIOSCIENCES COMPANY LIMITED, Shenzhen (CN); SHENZHEN JUMAI BIOTECHNOLOGY CO., LTD., Shenzhen (CN)

(72) Inventors: Lei SUN, Shenzhen (CN); Huan SHANG, Shenzhen (CN); Bo YANG, Shenzhen (CN); Mingliang YANG, Shenzhen (CN); Jinsen CAI, Shenzhen (CN); Yongzhi LI, Shenzhen (CN)

(73) Assignees: GENEMIND BIOSCIENCES COMPANY LIMITED, Shenzhen (CN); SHENZHEN JUMAI BIOTECHNOLOGY CO., LTD., Shenzhen (CN)

(21) Appl. No.: 19/444,212

(22) Filed: Jan. 9, 2026

(30) Foreign Application Priority Data
Jan. 24, 2025 (CN) 202510124960.7

(51) Int. Cl. C12Q 1/6874; C12Q 1/6804; G01N 21/64

(52) U.S. Cl. CPC C12Q 1/6874; C12Q 1/6804; G01N 21/6428; G01N 21/6458; G01N 2021/6439

(57) ABSTRACT

The present application presents a sequencing primer and a sequencing method. The sequencing primer comprises: a first nucleic acid segment, the first nucleic acid segment having a reactive group at the 3′ end; a second nucleic acid segment; a linker, the linker being bound to the 5′ end of the first nucleic acid segment and the 3′ end of the second nucleic acid segment, respectively, or the linker being bound to the 5′ end of the first nucleic acid segment and the 5′ end of the second nucleic acid segment, respectively, and when the linker is bound to the 5′ end of the first nucleic acid segment, the first nucleic acid segment having a 3′ occluded end. The primer has a three-segment structure, the first and third segments binding to a target molecule, and the second segment having a linking and bridging effect, so as to span a spatial distance, such that the sequencing primer has a more stable binding ability, and the accuracy of a sequencing result is improved.

### CROSS-REFERENCE TO RELATED APPLICATION

This application claims priority to Chinese Patent Application No. 202510124960.7, filed on Jan. 24, 2025, the entire contents of each of which are hereby incorporated by reference

### SEQUENCE LISTING

The instant application contains a Sequence Listing which has been submitted electronically in XML format and is hereby incorporated by reference in its entirety. The XML copy, created on Feb. 9, 2026, is named “2026 Feb. 9-Sequence Listing-20969-D023US00” and is 6,580 bytes in size.

### TECHNICAL FIELD

The present invention relates to the field of sequencing. In particular, the present application relates to a sequencing primer and a sequencing method.

### BACKGROUND

Next-Generation Sequencing (NGS), also known as high-throughput sequencing or massively parallel sequencing, was originally developed on the basis of the principle of pyrosequencing. With sequencing by synthesis as a basic design concept, and using a nucleic acid to be determined as a template, NGS allows introduction of specific nucleotides by means of a series of base extension reactions, which in turn allows completion of sequence determination of the nucleic acid to be determined on the basis of the type of the nucleotides introduced.

Because of the ability to simultaneously determine a large number of target genes and variation sites thereof at one time, high-throughput sequencing has high sensitivity and specificity of determination, allows both qualitative and quantitative determination, and has relatively low costs of determination for the same number of genes and sites. Thus, NGS exhibits very broad prospects of clinical and scientific research applications in fields such as noninvasive prenatal screening (NIPS), tumor gene mutations, genetic diseases, preimplantation genetic screening (PGS) and preimplantation genetic diagnosis (PGD), pathogenic microorganisms, and metagenomics. NGS has become the current most efficient instrument for DNA and RNA sequence analysis, and is also a supporting technology for research and for clinical disease diagnosis and treatment in the era of precision medicine.

In a conventional sequencing process, a primer is used as a substrate to hybridize with a nucleic acid template, and nucleotides or analogs are incorporated into the 3′ end of the primer strand by means of a polymerase, to realize the extension of the primer strand, thereby synthesizing a new strand complementary to the nucleic acid template. Thus, the stability of binding of the primer to the nucleic acid template directly affects the progress of the sequencing reaction. Increasing the length of the primer and performing base modification on the primer can improve the stability of binding of the primer to the nucleic acid molecule. However, increasing the length of the primer will increase the Tm value of the primer in a sequencing reagent, which is not conductive to the progress of the sequencing reaction. Modification of a base in the primer will significantly increase the cost of the primer.

Therefore, it is very important to find a primer capable of improving the stability of binding to a nucleic acid molecule.

### SUMMARY

The present invention aims to resolve one of the described technical problems to at least a certain extent, or at least provide a practical commercial option. For this purpose, an object of the present invention is to present a primer capable of improving the stability of binding to a nucleic acid molecule.

In one aspect of the present application, the present application presents a sequencing primer, comprising:
a first nucleic acid segment, the first nucleic acid segment having a reactive group at the 3′ end, the reactive group having reactivity to undergo a polymerization reaction with a nucleotide or an analog thereof, and the first nucleic acid segment being configured to specifically bind to at least a portion of a sequence within a first region at the 3′ end of a target nucleic acid molecule; a second nucleic acid segment, the second nucleic acid segment being configured to specifically bind to at least a portion of a sequence within a second region of a target nucleic acid molecule; and a linker, the linker being bound to the 5′ end of the first nucleic acid segment and the 3′ end of the second nucleic acid segment, respectively, or the linker being bound to the 5′ end of the first nucleic acid segment and the 5′ end of the second nucleic acid segment, respectively, and when the linker is bound to the 5′ end of the first nucleic acid segment, the first nucleic acid segment having a 3′ occluded end.

According to embodiments of the present disclosure, the sequencing primer (tripartite primer) consists of three parts, i.e., two nucleic acid segments and a linker, the two nucleic acid segments being located at two ends of the linker, respectively. When the sequencing primer is used to perform sequencing, the first nucleic acid segment, as a sequencing primer, hybridizes with the first region of the target nucleic acid molecule, and on that basis, the linker can span over a sequence region, such that the second nucleic acid segment at the 3′ end thereof hybridizes with the second region in the target nucleic acid molecule, thereby improving the binding stability between the primer and the target nucleic acid molecule, without modifying the base and adjusting conditions for the sequencing. Therefore, the target nucleic acid molecule using the sequencing primer can be subjected to a sequencing reaction in a harsher environment. Specifically, rapid sequencing can be performed under reaction conditions of stronger alkalinity and/or higher temperature, and a higher accuracy rate can be obtained.

In addition, the inventors have surprisingly found that, for a cluster of nucleic acid templates that each comprise a first inserted nucleic acid fragment and a second inserted nucleic acid fragment, when the sequencing primer is used as a sequencing primer for the first inserted nucleic acid fragment, since the 3′ end of the second nucleic acid segment in the sequencing primer does not have a reactive group, a portion of the second inserted nucleic acid fragments cannot be extended on the basis of the second nucleic acid segment, such that in the cluster of nucleic acid templates, there exists a difference between the number of the first inserted nucleic acid fragments capable of undergoing an extension reaction and the number of the second inserted nucleic acid fragments capable of undergoing an extension reaction. The difference can be used to obtain a difference in sequencing signals generated by the first inserted nucleic acid fragments and the second inserted nucleic acid fragments in each round of extension reaction, and hence, the first inserted nucleic acid fragments and the second inserted nucleic acid fragments can be synchronously sequenced on the basis of the signal difference.

As a possible embodiment of the present application, the reactive group comprises —OH.

As a possible embodiment of the present application, the linker binds to the 5′ end of the first nucleic acid segment through a first linking group, and the linker binds to the 3′ end or 5′ end of the second nucleic acid segment through a second linking group.

As a possible embodiment of the present application, the first linking group and/or the second linking group are each independently selected from a hydroxyl group, a carboxyl group, a sulfonate group, an ester group, a phosphate ester group, an amino group, an ether group, a saccharide group, an alkyl group, an amide group, an aromatic ring, or a biological macromolecule having a molecular weight of not more than 100,000.

As a possible embodiment of the present application, the biological macromolecule is selected from a polysaccharide, a polypeptide chain, and an oligomeric nucleic acid.

As a possible embodiment of the present application, the linker has a length of 0.3 nm to 100 nm.

As a possible embodiment of the present application, the linker comprises one or multiple hydrophilic groups.

As a possible embodiment of the present application, the hydrophilic group comprises at least one of a hydroxyl group, a carboxyl group, a sulfonate group, an ester group, a phosphate ester group, an amino group, a saccharide group, an ether group, and an amide group.

As a possible embodiment of the present application, the linker comprises at least one selected from an optionally substituted or unsubstituted C5-C20 alkylene, a C5-C20 alkylene in which one or more carbon atoms are substituted by a heteroatom, a small molecule compound, a polymer, a peptide chain, a polynucleotide, a polysaccharide, and an aromatic ring.

As a possible embodiment of the present application, the linker is selected from a polymer, the number of backbone atoms in the polymer being greater than or equal to 20 atoms, and a molecular weight thereof being greater than or equal to 2,000.

As a possible embodiment of the present application, the polymer comprises at least one of a hydroxyl group, a carboxyl group, a sulfonate group, an ester group, a phosphate ester group, an amino group, an ether group, an amide group, a saccharide group, and an alkyl group.

As a possible embodiment of the present application, the polymer comprises at least one selected from polyethylene glycol, polyvinyl alcohol, polyacrylamide, polymethacrylic acid, polyvinyl sulfonic acid, and polyvinylpyrrolidone; preferably, the polymer comprises at least one selected from iSp18 (polyethylene glycol 6), polyethylene glycol 200, polyethylene glycol 800, polyethylene glycol 2000, and polyethylene glycol 4000.

As a possible embodiment of the present application, the linker is selected from a nucleotide or polynucleotide composed of 1 to 40 bases, preferably a polynucleotide composed of 3 to 26 bases.

As a possible embodiment of the present application, the linker is selected from a small molecule compound the number of backbone atoms in which is greater than or equal to 6.

As a possible embodiment of the present application, in each sequencing primer, the molar ratio of the first nucleic acid segment to the second nucleic acid segment is 1:10 to 10:1.

As a possible embodiment of the present application, each sequencing primer comprises a number N of the first nucleic acid segments, and the N first nucleic acid segments are linked by means of 1 to N of the linkers, N being a positive integer less than or equal to 10.

As a possible embodiment of the present application, each sequencing primer comprises a number M of the second nucleic acid segments, and the M second nucleic acid segments are linked by means of 1 to M of the linkers, M being a positive integer less than or equal to 10.

As a possible embodiment of the present application, each sequencing primer comprises N of the first nucleic acid segments, and the N first nucleic acid segments are linked by means of one linker, N being a positive integer less than or equal to 10,
and each sequencing primer comprises M of the second nucleic acid segments, and the M second nucleic acid segments are linked by means of one linker, M being a positive integer less than or equal to 10.

In a possible embodiment of the present application, MEN; preferably, M=N=1.

As a possible embodiment of the present application, each sequencing primer comprises N of the first nucleic acid segments, and the N first nucleic acid segments are linked to the linker by means of N first linking groups, respectively, N being a positive integer less than or equal to 10;
and each sequencing primer comprises M of the second nucleic acid segments, and the M second nucleic acid segments are linked by means of M of the linkers, respectively, M being a positive integer less than or equal to 10.

In a possible embodiment of the present application, MEN; preferably, M=N=1.

As a possible embodiment of the present application, each sequencing primer comprises N of the first nucleic acid segments, and the N of the first nucleic acid segments are linked to one linker by means of N of the first linking groups, respectively, N being a positive integer less than or equal to 10;
and each sequencing primer comprises M of the second nucleic acid segments, and the M of the second nucleic acid segments are sequentially linked by means of M of the linkers, M being a positive integer less than or equal to 10,
and the first nucleic acid segments are bound to the linker at the 3′ end of the sequencing primer.

In a possible embodiment of the present application, MEN; preferably, M=N=1.

As a possible embodiment of the present application, each sequencing primer comprises one first nucleic acid segment;
and each sequencing primer comprises M of the second nucleic acid segments, and the M of the second nucleic acid segments are sequentially linked by means of M of the linkers, M being a positive integer less than or equal to 10,
and the first nucleic acid segments are bound to the linker at the 3′ end of the sequencing primer.

As a possible embodiment of the present application, each sequencing primer comprises N of the first nucleic acid segments, and the N of the first nucleic acid segments are linked to the linker by means of N first linking groups, respectively, N being a positive integer less than or equal to 10;
and each sequencing primer comprises M of the second nucleic acid segments, and the M of the second nucleic acid segments are linked by means of M of the linkers, respectively, M being a positive integer less than or equal to 10.

As a possible embodiment of the present application, each sequencing primer comprises N of the first nucleic acid segments, and the N of the first nucleic acid segments are linked to the linker by means of N first linking groups, respectively, N being a positive integer less than or equal to 10;
and each sequencing primer comprises M of the second nucleic acid segments, and the M of the second nucleic acid segments are linked to one linker by means of M second linking groups, respectively, M being a positive integer less than or equal to 10.

As a possible embodiment of the present application, each sequencing primer consists of one first nucleic acid molecule, one linker, and one second nucleic acid molecule.

As a possible embodiment of the present application, the target nucleic acid molecule comprises an inserted nucleic acid fragment, and a first region and a second region located at the 3′ end of the inserted nucleic acid fragment.

As a possible embodiment of the present application, a spacer sequence is provided between the first region and the second region.

As a possible embodiment of the present application, the spacer sequence comprises a tag sequence.

As a possible embodiment of the present application, the spacer sequence has a length of 3 nt to 26 nt.

As a possible embodiment of the present application, the first nucleic acid segment is configured to specifically bind to the first region of the sequencing library molecules; and/or the second nucleic acid segment is configured to specifically bind to the second region of the sequencing library molecules.

As a possible embodiment of the present application, in each sequencing primer, the number of first nucleic acid segments is greater than or equal to the number of second nucleic acid segments.

As a possible embodiment of the present application, each sequencing primer comprises a number X of first nucleic acid segments, and the X of the first nucleic acid segments specifically bind to the first regions of X of the target nucleic acid molecules, respectively, X being a positive integer less than or equal to 10.

As a possible embodiment of the present application, in each sequencing primer, the number of the first nucleic acid segments is equal to the number of the second nucleic acid segments.

As a possible embodiment of the present application, in each sequencing primer, the number of the first nucleic acid segments and the number of the second nucleic acid segments are both 1.

As a possible embodiment of the present application, the linker comprises at least one selected from an optionally substituted or unsubstituted C5-C20 alkylene, a C5-C20 alkylene in which one or more carbon atoms are substituted by a heteroatom, a polymer, and a polynucleotide.

As a possible embodiment of the present application, the linker is a polymer, and the polymer has a length of 300 or more atoms and a molecular weight greater than or equal to 4,000.

As a possible embodiment of the present application, the polymer comprises at least one of a hydroxyl group, a carboxyl group, a sulfonate group, an ester group, a phosphate ester group, an amino group, an ether group, a saccharide group, an alkyl group and an amide group.

As a possible embodiment of the present application, the polymer comprises at least one selected from polyethylene glycol, polyvinyl alcohol, polyacrylamide, polymethacrylic acid, polyvinyl sulfonic acid, and polyvinylpyrrolidone.

As a possible embodiment of the present application, the polymer is iSp18, polyethylene glycol 200, polyethylene glycol 800, polyethylene glycol 2000 or polyethylene glycol 4000.

As a possible embodiment of the present application, the length of the linker is greater than the length of the spacer sequence.

As a possible embodiment of the present application, the linker has a length of 0.3 nm to 100 nm.

As a possible embodiment of the present application, the linker is a polynucleotide, and the polynucleotide has a length of 1 nt to 40 nt, preferably 4 nt to 20 nt.

As a possible embodiment of the present application, there are multiple target nucleic acid molecules, and the multiple target nucleic acid molecules are derived from the same cluster of nucleic acid molecule clones, the target nucleic acid molecules each comprising a first inserted nucleic acid fragment and a second inserted nucleic acid fragment, the second inserted nucleic acid fragment being located at the 5′ end of the first inserted nucleic acid fragment; the 3′ end of the first inserted nucleic acid fragment is provided with a first primer binding site, and the first region and the second region are the first primer binding sites of different target nucleic acid molecules.

As a possible embodiment of the present application, a second primer binding site is provided between the first inserted nucleic acid fragment and the second inserted nucleic acid fragment.

As a possible embodiment of the present application, in each sequencing primer, the number of first nucleic acid segments is greater than, equal to or less than the number of second nucleic acid segments.

As a possible embodiment of the present application, the number of first nucleic acid segments is greater than, equal to or less than the number of second nucleic acid segments.

As a possible embodiment of the present application, the molar ratio of first nucleic acid segments to second nucleic acid segments is 1:1 to 1:5.

As a possible embodiment of the present application, the linker comprises at least one selected from an optionally substituted or unsubstituted C5-C20 alkylene, a C5-C20 alkylene in which one or more carbon atoms are substituted by a heteroatom, a polymer, and a polynucleotide.

As a possible embodiment of the present application, the linker is a polymer, and the polymer has a length of 300 or more atoms and a molecular weight greater than or equal to 4,000.

As a possible embodiment of the present application, the polymer comprises at least one of a hydroxyl group, a carboxyl group, a sulfonate group, an ester group, a phosphate ester group, an amino group, an ether group, and an amide group.

As a possible embodiment of the present application, the polymer comprises at least one selected from polyethylene glycol, polyvinyl alcohol, polyacrylamide, polymethacrylic acid, polyvinyl sulfonic acid, and polyvinylpyrrolidone.

As a possible embodiment of the present application, the polymer is iSp18, polyethylene glycol 200, polyethylene glycol 800, polyethylene glycol 2000 or polyethylene glycol 4000.

As a possible embodiment of the present application, the linker has a length of 0.3 nm to 100 nm.

As a possible embodiment of the present application, the linker has a length of 1 nt to 40 nt, preferably 4 nt to 20 nt.

In another aspect of the present application, the present application presents two sequencing methods. The first sequencing method comprises: (a1) hybridizing a sequencing primer to a nucleic acid template to form a primer-nucleic acid template complex, wherein the nucleic acid template comprises an inserted nucleic acid fragment, and a sequencing primer binding site and a known sequence located at the 3′ end of the inserted nucleic acid fragment, and the known sequence is located at the 3′ end of the sequencing primer binding site, and a spacer sequence is provided between the sequencing primer binding site and the known sequence; the sequencing primer comprises a linker, a first nucleic acid segment and a second nucleic acid segment, two ends of the linker binding to the 5′ end of the first nucleic acid segment and the 3′ end of the second nucleic acid segment, respectively, and the 3′ end of the first nucleic acid segment having a reactive group, the reactive group having reactivity to undergo a polymerization reaction with a nucleotide or an analog thereof; in the primer-nucleic acid template complex, the first nucleic acid segment is partially complementary or completely complementary to the sequencing primer binding site, the second nucleic acid segment is partially complementary or completely complementary to the known sequence of the nucleic acid template, and the sequence of the first nucleic acid segment is different from the sequence of the second nucleic acid segment;
(b1) Contacting one of the following substrates or substrate combinations i) to iii) and a polymerase with the primer-nucleic acid template complex, such that the substrate or substrate combination is bound to the polymerase and the primer-nucleic acid template complex, and/or the substrate or substrate combination is incorporated into the 3′ end of the primer strand of the primer-nucleic acid template complex,
i) A first nucleotide, the first nucleotide comprising a fluorescent label and a blocking group used to prevent the incorporation of a next nucleotide or an analog thereof into the primer strand,
ii) A second nucleotide, the second nucleotide being nucleotide containing a fluorescent label, or an analog thereof, and
iii) A third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand;
(c1) Exciting the fluorescent label to generate a fluorescence signal, and collecting a fluorescence image; and
(e1) Repeating step (b1) to step (c1) one or more times.

The first sequencing method can improve the binding stability between the sequencing primer and the nucleic acid template. Therefore, the target nucleic acid molecule using the sequencing primer can undergo a sequencing reaction in a harsher environment. Specifically, rapid sequencing can be performed under reaction conditions of stronger alkalinity and/or higher temperature, and a higher accuracy rate can be obtained.

As a possible embodiment of the present application, in step (b1), the substrate is i), and the first nucleotide, the polymerase and the primer-nucleic acid template complex are contacted under conditions suitable for a polymerization reaction to occur, such that the first nucleotide is incorporated into the 3′ end of the primer strand of the primer-nucleic acid template complex;
Step (c1) further comprises: determining, based on the fluorescence image, the type of the nucleotide or the analog incorporated into the primer strand.

As a possible embodiment of the present application, in step (b1), the substrate is ii), and the second nucleotide, the polymerase and the primer-nucleic acid template complex are contacted under non-polymerization reaction conditions, such that the second nucleotide is bound to the polymerase and the primer-nucleic acid template complex;
Step (c1) further comprises: determining, based on the fluorescence image, the type of the nucleotide or the analog thereof bound to the polymerase;

After step (c1), further comprised is:
(d1) Removing the second nucleotide; contacting the third nucleotide, the polymerase and the primer-nucleic acid template complex under conditions suitable for a polymerization reaction, so as to incorporate the third nucleotide into the 3′ end of the primer strand of the primer-nucleic acid template complex.

As a possible embodiment of the present application, contacting under non-polymerization reaction conditions comprises:
Contacting under conditions in which Mg²⁺ and/or Mn²⁺ are not contained;
Contacting under conditions in which at least one of Ca²⁺, Ni²⁺ and Si²⁺ is contained.

As a possible embodiment of the present application, removing the second nucleotide comprises:
Adding a chelating agent and performing rinsing, to remove the second nucleotide; and/or
Adding Mg²⁺ and/or Mn²⁺, and the third nucleotide.

As a possible embodiment of the present application, in step (b1), the substrate is iii), and the third nucleotide, the fluorescently labeled antibody, the polymerase and the primer-nucleic acid template complex are contacted under conditions suitable for a polymerization reaction, such that a nucleotide or an analog thereof having a blocking group is incorporated into the 3′ end of the primer strand of the primer-nucleic acid template complex, and the fluorescently labeled antibody is bound to the third nucleotide;
Step (c1) further comprises: determining, based on the fluorescence image, the type of the nucleotide or the analog thereof bound to the polymerase.

As a possible embodiment of the present application, after step (c1), further comprised is:
(d1) Removing the fluorescently labeled antibody.

As a possible embodiment of the present application, in step (b1), the conditions suitable for a polymerization reaction to occur include one or two of the following conditions 1) and 2):
1) A reaction temperature of 30° C. to 75° C.; and
2) A pH of 8.0 to 10.

As a possible embodiment of the present application, the conditions of for a polymerization reaction include: a pH of 9.0 to 10.0, and/or a temperature of 50° C. to 75° C.

As a possible embodiment of the present application, the conditions of for a polymerization reaction include: a pH of 9.5 to 10.0 and/or a temperature of 55° C. to 75° C.

As a possible embodiment of the present application, the sequence of the sequencing primer binding site is different from the sequence of the known sequence.

In a possible embodiment of the present application, the known sequence is a first adaptor.

As a possible embodiment of the present application, the spacer sequence comprises a tag sequence.

As a possible embodiment of the present application, the spacer sequence has a length of 3 nt to 26 nt.

As a possible embodiment of the present application, in each sequencing primer, the number of first nucleic acid segments is greater than or equal to the number of second nucleic acid segments.

As a possible embodiment of the present application, when the sequencing primer comprises multiple first nucleic acid segments and/or multiple second nucleic acid segments, the multiple first nucleic acid segments are linked by means of the same linker, and the multiple second nucleic acid segments are linked by means of the same linker.

As a possible embodiment of the present application, when the sequencing primer comprises multiple first nucleic acid segments and multiple second nucleic acid segments, the multiple first nucleic acid segments are linked to the second nucleic acid segments by means of multiple linkers, respectively, and each of the multiple linkers is linked to at least one of the other linkers.

As a possible embodiment of the present application, each sequencing primer comprises X first nucleic acid segments, and the X first nucleic acid segments are partially complementary or completely complementary to the sequencing primer binding sites of X nucleic acid templates, respectively, X being a positive integer greater than 1 and less than 10.

As a possible embodiment of the present application, in each sequencing primer, the number of the first nucleic acid segments is equal to the number of the second nucleic acid segments.

As a possible embodiment of the present application, in each sequencing primer, the number of the first nucleic acid segments and the number of the second nucleic acid segments are both 1.

As a possible embodiment of the present application, the linker comprises at least one selected from an optionally substituted or unsubstituted C5-C20 alkylene, a C5-C20 alkylene in which one or more carbon atoms are substituted by a heteroatom, a polymer, and a polynucleotide.

As a possible embodiment of the present application, the linker is selected from a polymer, and the number of backbone atoms in the linker is greater than or equal to 300 atoms, and a molecular weight thereof is greater than or equal to 4,000.

As a possible embodiment of the present application, the polymer comprises at least one of a hydroxyl group, a carboxyl group, a sulfonate group, an ester group, a phosphate ester group, an amino group, an ether group, and an amide group.

As a possible embodiment of the present application, the polymer comprises at least one selected from polyethylene glycol, polyvinyl alcohol, polyacrylamide, polymethacrylic acid, polyvinyl sulfonic acid, and polyvinylpyrrolidone.

As a possible embodiment of the present application, the linker is a polymer, and the polymer is iSp18, polyethylene glycol 200, polyethylene glycol 800, polyethylene glycol 2000 or polyethylene glycol 4000.

As a possible embodiment of the present application, the linker is a polynucleotide, and the length of the polynucleotide is greater than the length of the spacer sequence.

As a possible embodiment of the present application, the linker has a length of 0.3 nm to 100 nm.

As a possible embodiment of the present application, the linker is a polynucleotide, and the polynucleotide has a length of 1 nt to 40 nt, preferably 4 nt to 20 nt.

As a possible embodiment of the present application, the polynucleotide has a GC content of 0% to 70%.

As a possible embodiment of the present application, the nucleic acid template is fixed on a solid phase substrate by means of the 5′ end.

As a possible embodiment of the present application, a cluster of clones formed of the nucleic acid templates is provided on the solid phase substrate, and the nucleic acid templates each at least comprise, from the 3′ end to the 5′ end: a known sequence, a spacer sequence, a sequencing primer binding site, and an inserted nucleic acid fragment.

The second sequencing method comprises: (a2) hybridizing a first sequencing primer and a second sequencing primer to a cluster of nucleic acid templates to form primer-nucleic acid template complexes, wherein the cluster of nucleic acid templates at least comprises a first nucleic acid template and a second nucleic acid template, the first nucleic acid template and the second nucleic acid template each comprise a first inserted nucleic acid fragment and a second inserted nucleic acid fragment, and a first primer binding site and a second primer binding site located at the 3′ end of the first inserted nucleic acid fragment and the 3′ end of the second inserted nucleic acid fragment, respectively, and the first inserted nucleic acid fragment is located at the 3′ end of the second inserted nucleic acid fragment; the first sequencing primer comprises a linker, a first nucleic acid segment and a second nucleic acid segment, two ends of the linker are bound to the 5′ end of the first nucleic acid segment and the 3′ end or 5′ end of the second nucleic acid segment, respectively, and the 3′ end of the first nucleic acid segment has a reactive group, the reactive group having reactivity to undergo a polymerization reaction with a nucleotide or an analog thereof, and when the linker binds to the 5′ end of the second nucleic acid segment, the second nucleic acid segment has a 3′ occluded end; in the primer-nucleic acid template complexes, the first nucleic acid segment is partially complementary or completely complementary to the first primer binding site of the first nucleic acid template, the second nucleic acid segment is partially complementary or completely complementary to the first primer binding site of the second nucleic acid template, the second sequencing primer is partially complementary or completely complementary to the second primer binding site of the nucleic acid templates, and the sequence of the first nucleic acid segment is identical or complementary to the sequence of the second nucleic acid segment; the 3′ end of the second sequencing primer has a reactive group;
(b2) Contacting one of the following substrates or substrate combinations i) to iii) and a polymerase with the primer-nucleic acid template complexes, such that the substrate or substrate combination is bound to the polymerase and the primer-nucleic acid template complexes, and/or the substrate or substrate combination is incorporated into the 3′ end of the primer strand of the primer-nucleic acid template complexes,
iv) A first nucleotide, the first nucleotide comprising a fluorescent label, and a blocking group used to prevent the incorporation of a next nucleotide or an analog thereof into the primer strand,
v) A second nucleotide, the second nucleotide being a nucleotide containing a fluorescent label or an analog thereof, and

A third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand;
(c2) Exciting the fluorescently labeled antibody or nucleotide to generate a fluorescence signal, and collecting a fluorescence image; and
(f2) Repeating step (b2) to step (c2) one or more times.

In the second sequencing method, the sequencing binding sites for some of the first inserted nucleic acid fragments are blocked by using the second nucleic acid segment, causing the numbers of the extendable first sequencing primer and second sequencing primer to be different, such that the sequencing signals of the first inserted nucleic acid fragments become different from those of the second inserted nucleic acid fragments, and the first inserted nucleic acid fragments and the second inserted nucleic acid fragments are synchronously sequenced on the basis of the difference.

As a possible embodiment of the present application, in step (b2), the fluorescently labeled antibody substrate is i), and the first nucleotide, the polymerase and the primer-nucleic acid template complexes are contacted under conditions suitable for a polymerization reaction to occur, such that the first nucleotide is incorporated into the 3′ end of the primer strand of the primer-nucleic acid template complexes;
Step (c2) further comprises: determining, based on the fluorescence image, the type of the nucleotide or the analog incorporated into the primer strand.

As a possible embodiment of the present application, in step (b2), the substrate is ii), and the second nucleotide, the polymerase and the primer-nucleic acid template complexes are contacted under non-polymerization reaction conditions, such that the second nucleotide is bound to the polymerase and the primer-nucleic acid template complexes;
Step (c2) further comprises: determining, based on the fluorescence image, the type of the nucleotide or the analog thereof bound to the polymerase;
After step (c2), further comprised is:
(d2) Removing the second nucleotide; contacting the third nucleotide, the polymerase and the primer-nucleic acid template complex under conditions suitable for a polymerization reaction, so as to incorporate the third nucleotide into the 3′ end of the primer strand of the primer-nucleic acid template complex.

As a possible embodiment of the present application, contacting under non-polymerization reaction conditions comprises:
Contacting under conditions in which Mg²⁺ and/or Mn²⁺ are not contained;
Contacting under conditions in which at least one of Ca²⁺, Ni²⁺, and Si²⁺ is contained.

As a possible embodiment of the present application, removing the second nucleotide comprises:
Adding a chelating agent and performing rinsing, to remove the second nucleotide; and/or
adding Mg²⁺ and/or Mn²⁺, and the third nucleotide.

As a possible embodiment of the present application, in step (b1), the substrate is iii), and the third nucleotide, the fluorescently labeled antibody, the polymerase and the primer-nucleic acid template complex are contacted under conditions suitable for a polymerization reaction, such that a nucleotide or an analog thereof having a blocking group is incorporated into the 3′ end of the primer strand of the primer-nucleic acid template complex, and the fluorescently labeled antibody is bound to the third nucleotide;
Step (c2) further comprises: determining, based on the fluorescence image, the type of the nucleotide or the analog thereof bound to the polymerase.

As a possible embodiment of the present application, after step (c2), further comprised is:
(d2) Removing the fluorescently labeled antibody.

As a possible embodiment of the present application, in each sequencing primer, the molar ratio of the first nucleic acid segment to the second nucleic acid segment is 1:5 to 5:1.

As a possible embodiment of the present application, each first sequencing primer comprises N first nucleic acid segments, and the N first nucleic acid segments are linked by means of one linker, N being a positive integer less than or equal to 10.

As a possible embodiment of the present application, each first sequencing primer comprises N first nucleic acid segments, the 5′ end of the first nucleic acid segments has a first linking group, and the N first nucleic acid segments are linked to one linker by means of N first linking groups, respectively.

As a possible embodiment of the present application, each first sequencing primer comprises M second nucleic acid segments, and the M second nucleic acid segments are sequentially linked by means of 1 to M linkers, M being a positive integer less than or equal to 10.

As a possible embodiment of the present application, the linker is bound to the 5′ end of the first nucleic acid segment through a first linking group, and the linker is bound to the 3′ end of the second nucleic acid segment through a second linking group.

As a possible embodiment of the present application, each first sequencing primer comprises M second nucleic acid segments, and the M second nucleic acid segments are sequentially linked by means of M linkers, respectively, M being a positive integer less than or equal to 10.

As a possible embodiment of the present application, M=N=1.

As a possible embodiment of the present application, conditions for a polymerization reaction comprise: a pH of 8.5 to 10.0, and/or a temperature of 50° C. to 75° C.

As a possible embodiment of the present application, conditions for a polymerization reaction comprise: a pH of 9.8 to 10.0, and/or a temperature of 65° C. to 75° C.

As a possible embodiment of the present application, the linker comprises at least one selected from an optionally substituted or unsubstituted C5-C20 alkylene, a C5-C20 alkylene in which one or more carbon atoms are substituted by a heteroatom, a polymer, and a polynucleotide.

As a possible embodiment of the present application, the linker is selected from a polymer, and the number of backbone atoms in the linker is greater than or equal to 300 atoms, and a molecular weight thereof is greater than or equal to 4,000.

As a possible embodiment of the present application, the polymer comprises at least one of a hydroxyl group, a carboxyl group, a sulfonate group, an ester group, a phosphate ester group, an amino group, an ether group, and an amide group.

As a possible embodiment of the present application, the polymer comprises at least one selected from polyethylene glycol, polyvinyl alcohol, polyacrylamide, polymethacrylic acid, polyvinyl sulfonic acid, and polyvinylpyrrolidone.

As a possible embodiment of the present application, the polymer comprises at least one selected from iSp18, polyethylene glycol 200, polyethylene glycol 800, polyethylene glycol 2000 and polyethylene glycol 4000.

As a possible embodiment of the present application, the linker has a length of 0.3 nm to 100 nm.

As a possible embodiment of the present application, the linker has a length of 1 nt to 40 nt, preferably 4 nt to 20 nt.

As a possible embodiment of the present application, after step (c2) or before step (f2), further comprised is: determining the base type of the nucleotide incorporated or bound to the first sequencing primer strand and the second sequencing primer strand, respectively.

As a possible embodiment of the present application, determining the base type of the nucleotide incorporated or bound to the first sequencing primer strand and the second sequencing primer strand, respectively, comprises: on the basis of the sequencing signals at one or multiple specified positions in the fluorescence image, determining, in the sequencing signals, a first sequencing signal component derived from the first sequencing primer strand and a second sequencing signal component derived from the second sequencing primer strand, respectively, wherein the one or multiple specified positions at least comprise a first position, the sequencing signals at the first position comprise two fluorescence signals of different colors, and the second sequencing signal component is greater than the first sequencing signal component;
assigning the fluorescence signals corresponding to the first sequencing signal component in the fluorescence image obtained in step (c2) to the fluorescence signals of the first inserted nucleic acid fragment, and assigning the fluorescence signals corresponding to the second sequencing signal component to the fluorescence signals of the second inserted nucleic acid fragment; and
on the basis of the fluorescence signals to which the first sequencing signal component and the second sequencing signal component correspond, respectively, determining the base type of each nucleotide reversible terminator incorporated into the first sequencing primer strand and the second sequencing primer strand, respectively.

As a possible embodiment of the present application, the fluorescence signals are fluorescence color and fluorescence intensity.

Additional aspects and advantages of the present invention will be provided in part in the following description, and will become apparent in part from the following description, or may be understood by means of putting the present invention into practice.

### BRIEF DESCRIPTION OF THE DRAWINGS

The foregoing and/or additional aspects and advantages of the present invention will become apparent and readily understandable from the description of examples with reference to the following drawings, in which:

FIG. 1 shows a schematic structural diagram of a sequencing primer according to examples of the present invention;

FIG. 2 shows a schematic structural diagram of a target nucleic acid molecule according to examples of the present invention;

FIG. 3 shows a schematic structural diagram of the target nucleic acid molecule of FIG. 2 after hybridization with a sequencing primer;

FIG. 4 shows a structural diagram of another target nucleic acid molecule according to examples of the present disclosure;

FIG. 5 shows a schematic structural diagram of the target nucleic acid molecule of FIG. 4 after hybridization with a sequencing primer;

FIG. 6 shows a schematic structural diagram of the target nucleic acid molecule of FIG. 4 after hybridization with another sequencing primer;

FIG. 7 shows a schematic structural diagram of a nucleic acid template;

FIGS. 8A-8C show schematic structural diagrams of the tripartite primer according to Example 1 and the single primer of the Comparative Example of the present invention after hybridization with a nucleic acid template;

FIG. 9 shows a schematic diagram of a sequencing chip according to an embodiment of the present disclosure;

FIGS. 10A-10C show schematic structural diagrams of the tripartite primer of Example 2 and the single primer of the Control Example of the present invention after hybridization with a nucleic acid template;

FIG. 11 shows a statistical curve diagram of fluorescence signal brightness of the 11th to 20th rounds of sequencing of Example 2 and Control Example of the present invention;

FIG. 12 shows fluorescent images of G base obtained in the 5th round of sequencing in Example 2 and Control Example of the present invention; and

FIG. 13 shows the curves of florescence signal intensity change of 200 rounds of sequencing in Example 2 and the Control Example of the present invention.

### DETAILED DESCRIPTION

In order to make the technical problems to be solved, technical solutions and beneficial effects of the present application clearer and more understandable, the present application will be further described in detail below in conjunction with examples. It should be understood that the specific examples described herein are merely used to explain the present application, and are not intended to limit the present application.

The term “at least one” refers to one or multiple, and “multiple” refers to two or more than two. “At least one (kind)” or a similar expression thereof refers to any combination of these items, including a single item (a single one) or any combination of multiple items (multiple ones). For example, “at least one (kind) of a, b, or c” or “at least one (kind) of a, b, and c” may both denote: a, b, c, a-b (i.e., a and b), a-c, b-c, or a-b-c, wherein a, b and c may each be a single item (a single one) or multiple items (multiple ones).

The singular forms of “a”, “said” and “the” used in examples of the present application and the appended claims are also intended to include plural forms, unless the context clearly indicates otherwise.

The terms “first” and “second” are used for descriptive purposes only, are used for distinguishing purposes such as positions, objects, and the like from one another, and should not be understood as indicating or implying relative importance or implicitly specifying the number of the indicated technical features. For example, without departing from the scope of embodiments of the present application, the first nucleic acid segment may be referred to as the second nucleic acid segment, and the second nucleic acid segment may be referred to as the first nucleic acid segment. Thus, features defined by “first” and “second” may explicitly or implicitly include one or more of the features.

In the description of the present application, the concentrations or contents mentioned for relevant components or substances may not only refer to the specific contents of the components or substances, but also represent proportional relationships among the contents of the components or substances. Therefore, either scaling up or scaling down according to the contents of the relevant components in the examples of the description of the present application would be within the scope disclosed in the examples of the description of the present application. The content may be content in mass percentage, content in molar concentration percentage, etc., and the content in percentage may be a specific content in percentage, or may be a relative content in percentage determined by using the content of a reference as a standard.

It should be understood that, in various examples of the present application, the sequence numbers of respective processes do not mean the order of execution, and some or all of the steps may be executed in parallel or sequentially. The order of execution of the respective processes should be determined by the function and internal logic thereof, and should not constitute any limitation on the execution process of examples of the present application.

The term “sequencing” used in embodiments of the present application may also be referred to as “nucleic acid sequencing” or “gene sequencing”. The three terms are semantically interchangeable, and all refer to determining the type and arrangement order of bases or nucleotides (including nucleotide analogs) in a nucleic acid molecule. Said sequencing comprises a process of binding nucleotides to a template and collecting the corresponding signals emitted from the nucleotides (including analogs). Said sequencing includes sequencing by synthesis (SBS) and/or sequencing by ligation (SBL), includes DNA sequencing and/or RNA sequencing, and includes long fragment sequencing and/or short fragment sequencing; long fragment and short fragment are in relative terms, e.g., nucleic acid molecules longer than 1 Kb, 2 Kb, 5 Kb or 10 Kb may be referred to as long fragments, and nucleic acid molecules shorter than 1 Kb or 800 bp may be referred to as short fragments.

Sequencing generally comprises multiple cycles of a process, to achieve determination of the types and arrangement order of multiple bases or nucleotides on a nucleic acid template. In examples of the present application, each cycle of the “process to achieve determination of the types and arrangement order of multiple bases or nucleotides on a nucleic acid template” is referred to as “one cycle of sequencing”. A “cycle”, also referred to as a “sequencing cycle”, can be defined as one instance of base extension of the four kinds of nucleotide/base. In other words, a “sequencing cycle” can be defined as completing the determination of the type of the base or nucleotide at any designated position on the template. For a sequencing platform that achieves sequencing based on a polymerization or ligation reaction, one cycle of sequencing comprises a process of achieving binding of the four kinds of nucleotide (including nucleotide analogs) to the so-called nucleic acid template in a base-complementary manner at one time, and collecting the corresponding signals that are emitted. For a platform that achieves sequencing based on a polymerization reaction, the reaction system comprises nucleotides as reaction substrates, a polymerase, and a nucleic acid template onto which a segment of sequence (sequencing primer) is bound. On the basis of the principle of base pairing and the mechanism of the polymerization reaction, an added nucleotide as a reaction substrate, catalyzed by the polymerase, is linked to the sequencing primer to achieve binding of the nucleotide to a particular position of the nucleic acid template. Generally, one cycle of sequencing may comprise one instance or multiple instances of base extension (repeat). For example, the four kinds of nucleotide are sequentially added to a reaction system, and base extension and collection of a corresponding reaction signal are performed for each; one cycle of sequencing comprises four instances of base extension. Alternatively, for example, any combination of the four kinds of nucleotide is added to a reaction system. For example, the four kinds of nucleotide are added in sets of two and two, or sets of one and three, and base extension and collection of corresponding reaction signals are performed for the two sets respectively; one cycle of sequencing comprises two instances of base extension. As another example, the four kinds of nucleotide are added to a reaction system at the same time for base extension and collection of reaction signals; one cycle of sequencing comprises one instance of base extension.

The term “nucleotide” refers to the four kinds of natural nucleotides (e.g., dATP, dCTP, dGTP and dTTP, or ATP, CTP, GTP and UTP) or derivatives thereof, and sometimes is also directly represented by the bases (A, T or U, C, and G) that they contain. The nucleotides or bases referred to by the shown expressions may be known to a person of ordinary skill in the art from the context. By the type of a nucleotide, what is meant is which of the described dATP, dCTP, dGTP and dTTP, or derivatives thereof, the nucleotide belongs, or to which of the described ATP, CTP, GTP and UTP, or derivatives thereof, the nucleotide belongs.

The term “nucleic acid molecule” means a polymeric form of nucleotides of any length, and may include ribonucleotides or analogs thereof, deoxyribonucleotides or analogs thereof, and mixtures formed of these nucleotides or analogs thereof. The nucleic acid molecule may refer to a single-stranded polynucleotide or a double-stranded polynucleotide. The nucleotides in a nucleic acid molecule may include naturally occurring nucleotides and functionally alternative analogs thereof. Examples of analogs are capable of hybridizing to a nucleic acid in a sequence-specific manner, or are capable of serving as a template for the replication of a particular nucleotide sequence. A naturally occurring nucleotide typically has a backbone comprising a phosphodiester bond. The structure of an analog may have a backbone linkage that includes any type of substitution known in the art. A naturally occurring nucleotide typically has a deoxyribose sugar (e.g., found in DNA) or a ribose sugar (e.g., found in RNA). The structure of an analog may have an alternative sugar moiety, including any type known in the art. A nucleotide may comprise a natural base or non-natural base. The bases in natural DNA may include one or more of adenine, thymine, cytosine, and/or guanine, and the bases of natural RNA may include one or more of adenine, uracil, cytosine, and/or guanine. A nucleotide can also use any non-natural base or base analog, such as a locked nucleic acid (LNA) and a bridged nucleic acid (BNA).

The term “nucleic acid template” refers to a master nucleic acid molecule, or a fragment of the master nucleic acid molecule, to which nucleotides or nucleotide analogs are bound through successive cycles of base extension. The so-called nucleic acid template may be the entire sequence of a nucleic acid molecule or a partial fragment of the nucleic acid molecule. The “nucleic acid template” referred to may be a nucleic acid fragment used as a template in an extension reaction in which sequencing by synthesis is performed. Since the bases added by the extension reaction and the sequencing template satisfy the base pairing principle, the sequence of the sequencing template can be determined by determining the type of base added in each cycle of extension reaction. The sequence of the nucleic acid template herein may generally include, but is not limited to, at least one of a target molecule sequence, a UMI sequence and a sample tag sequence, sometimes also referred to herein as a “target nucleic acid molecule”.

The term “cluster” refers to a position site on a solid phase substrate, such as a chip, which position site may, in general, contain multiple nucleic acid molecules, but typically the multiple nucleic acid molecules at the position site are derived from the same nucleic acid molecule, such as an amplification cluster of multiple nucleic acid molecules formed from bridge PCR of the same nucleic acid molecule.

The term “primer”, also known as “probe”, refers to: an oligonucleotide or nucleic acid molecule that can be hybridized with a target sequence of interest. In examples, a primer functions as a substrate onto which nucleotides can be polymerized by means of a polymerase. For example, the primer may serve as a starting point for DNA or RNA synthesis. For example, a sequencing primer can hybridize to a synthetic nucleic acid template strand so as to prime the synthesis of a new strand that is complementary to the synthetic nucleic acid template strand. The primer may comprise any combination of nucleotides or analogs thereof. In some examples, the primer is a single-stranded oligonucleotide or polynucleotide.

The term “target nucleic acid molecule” or “nucleic acid template” nucleic acid molecule may refer to a sequence to be analyzed.

In examples of the present application, the term “library” is a pool/collection of nucleic acid molecules comprising multiple target fragments/fragments to be determined, which are derived from nucleic acids of a sample to be determined. Typically, the derived target fragments/fragments to be determined are processed, e.g., by adding a known sequence to one or both ends of the target fragments, e.g., by adding a linker (sequencing linker), such that the library can be linked or fixed onto a chip, so as to be suitable for being loaded onto a sequencing platform for sequencing.

The term “reversible terminator” refers to a nucleotide or an analog thereof that is capable of blocking the formation of a covalent bond between the 3′ hydroxyl moiety in the carbon sugar of the nucleotide or the analog thereof and the 5′ phosphoric acid of another nucleotide or an analog thereof. The blocking moiety on the nucleotide or the analog thereof may be reversible, and thus, may be removed or modified to allow the 3′ hydroxyl group to form a covalent bond with the 5′ phosphate of another nucleotide. For example, a “reversible terminator” may include a blocking moiety located, for example, at the 3′ position of a nucleotide, and may be a chemically cleavable moiety, such as allyl, azidomethyl, or methoxymethyl, or may be a group that may be enzymatically cleavable, such as a phosphate ester. The “reversible terminator” may or may not have a detectable label.

In examples of the present application, the term “bright spot” (spots or peaks), also referred to as a “bright point” or a “light point”, refers to a location on an image that has a relatively strong signal. For example, the signal at the location is stronger than that of a surrounding area, and appears as a relatively bright spot or point on the image. One bright spot or one location occupies one or multiple pixels. The signal at the bright spot/location may be from a target molecule or may be from a non-target substance.

In examples of the present application, the term “chip” refers to a solid phase substrate having a surface, such as a planar surface, to which biomolecules to be determined are linked, and is also referred to as a sequencing chip or a flow cell. The term “solid phase substrate” may also be referred to as a solid phase support, a sequencing chip or a sequencing biochip. In one example, the solid phase support is a substrate having an array of microwells, and each position corresponds to the position where one microwell lies. Illustratively, the solid phase support is a nanowell pattern chip, which comprises nanowells arranged in an array. The nanowells may also be referred to as nanopores, micropores, or holes. Nanowell and nanopore are interchangeable, and refer to a nano-sized porous structure formed on a surface of a solid support. The shape of the nanowell or nanopore is not strictly limited, and may be a circle, an ellipse, a triangle, a quadrangle or another polygon. The nanowell or nanopore may even have no particular shape. In order to facilitate the uniformity of the amplification of a nucleic acid template, in some examples, the nanowell or nanopore is configured to have a cross section having the shape of a symmetrical figure. Illustratively, the cross section of the nanowell or nanopore is a circle or a regular polygon. Examples of pattern chips include, for example, the pattern chip of Illumina, Inc., and the DNB (DNA nanoball) pattern chip of MGI Tech Co., Ltd. In one example, the solid phase support is a planar substrate, and a surface of the planar substrate comprises a plurality of surface-treated positions. An example of such a solid phase support is, for example, a random chip.

In examples of the present application, the term “channel” refers to channels formed in different ways during sequencing that are capable of screening and distinguishing between the four bases derived from A, C, G and T. The “channel” referred to in examples of the present application includes a four-channel scenario, which refers to four fluorescence signal optical channels formed during sequencing and imaging by using different kinds of excitation light, different fluorescent filters, etc., which are capable of screening and distinguishing between the four fluorescent bases derived from A, C, G and T/U (representing T or U). In actual sequencing, photographs will be taken in the four different fluorescence channels to form images. Ideally, each fluorescence channel has only the signal of the type of fluorescent base corresponding to the channel, but practically, due to the effect of fluorescence crosstalk, the fluorescence signals of other bases will appear in each channel in addition to the fluorescence signal of the corresponding fluorescent base. The “channel” referred to in examples of the present application also includes a double-channel scenario, which refers to fluorescence signal optical channels that use different kinds of excitation light, different fluorescent filters, etc. to screen and distinguish between fluorescence signals derived from fluorescent bases A, C, G and T/U each time, so as to obtain the four fluorescent bases A, C, G and T/U in two times. The “channel” referred to in examples of the present application also includes a single-channel scenario, which refers to fluorescence signal optical channels that use different signal intensities, two or more chemical reactions or imaging operations, etc., to screen and distinguish between fluorescence signals derived from bases A, C, G and T/U each time, so as to obtain the four fluorescent bases A, C, G and T/U. Of course, the number of channels is not limited by the above. The present application can also be used in sequencing methods using electrical signals, and the mechanism thereof is similar to the described methods using fluorescent signals.

In examples of the present application, “small molecule compound” refers to an organic compound having a molecular weight of less than 1,000 Daltons (Da).

In examples of the present application, “iSp18” refers to hexaethylene glycol, the main chain of which has a length of 18 atoms, that is, polyethylene glycol 6. In particular, iSp18 consists of 12 carbon atoms and 6 oxygen atoms, and can be used to introduce a relatively long spacer in an oligonucleotide. For example, iSp18 can be added to a DNA or RNA oligonucleotide to increase the length and flexibility thereof.

The degree of the stability of binding of a sequencing primer to a nucleic acid template affects the progress of nucleic acid sequencing. When the binding stability of the sequencing primer to the nucleic acid template is poor, the sequencing primer will easily detach from the nucleic acid template, such that the sequencing of the nucleic acid template cannot be performed normally due to loss of the primer. At present, most sequencing platforms perform a polymerization reaction at 50° C. to 60° C. and pH 8.5 to 9, and the binding stability of a sequencing primer to a nucleic acid template can be improved to a certain extent by reasonably configuring the length of the primer to reach a certain number of bases. With the development of sequencing applications, the demand for high-speed sequencing has gradually increased. Many researchers have sought to achieve a high sequencing speed by seeking harsh sequencing conditions, including seeking a high-speed sequencing reaction by increasing the reaction temperature, changing the pH condition, replacing with a new polymerase, etc., in the step of incorporating nucleotides or analogs thereof, i.e., the step of subjecting the nucleotides to a polymerization reaction. For example, the polymerization reaction is carried out in wider ranges of pH (in the range of from 8.5 to 10) and temperature (in the range of from 50° C. to 75° C.). However, as the reaction temperature and pH conditions increase, the double-stranded structure formed by the sequencing primer and the target nucleic acid molecule decreases steadily, and the sequencing primer will easily detach from the double-stranded structure, which is not conducive to the progress of sequencing. Compared with the chemical environment of low temperature and low pH, the detachment of the sequencing primer under high temperature and high pH conditions is significantly increased. In view of this, examples of the present application provide a new sequencing primer capable of improving the binding strength between the primer and the target nucleic acid molecule.

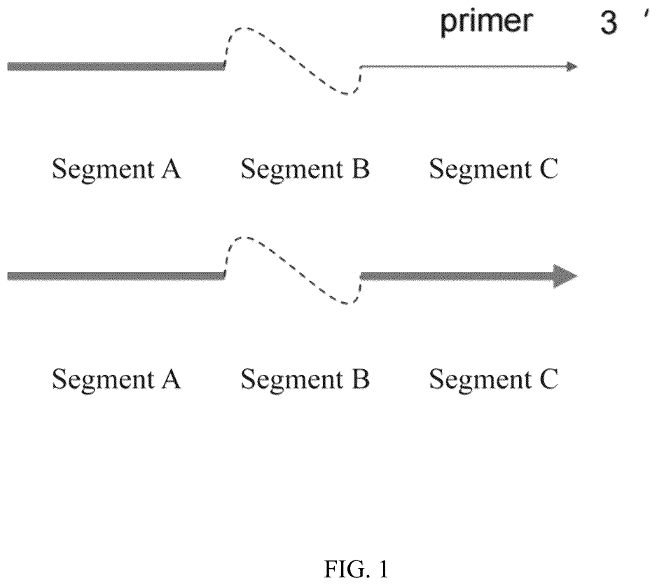

As shown in FIG. 1, the sequencing primer provided in examples of the present application comprises three parts, which are, respectively:
(1) A first nucleic acid segment (corresponding to segment C in FIG. 1).

The first nucleic acid segment is located at the 3′ end of the sequencing primer, and the 3′ end of the first nucleic acid segment has a reactive group. In examples of the present application, the reactive group at the 3′ end of the first nucleic acid segment has reactivity to undergo a polymerization reaction with a nucleotide or an analog thereof, such that the first nucleic acid segment can be extended at the 3′ end by means of the polymerization reaction. In some embodiments, the reactive group comprises —OH. In this case, the —OH at the 3′ end of the first nucleic acid segment reacts with a phosphate group of the nucleotide or the analog thereof to form a phosphate ester group, thereby achieving the extension of the nucleotide or the analog thereof. As part of the sequencing primer, the first nucleic acid segment is configured to specifically bind to at least a portion of a sequence within a first region at the 3′ end of a target nucleic acid molecule, and can undergo a polymerization reaction with the nucleotide or the analog thereof by means of the reactive group at the 3′ end, to achieve extension of the primer strand from the 5′ end to the 3′ end.
(2) A second nucleic acid segment. The second nucleic acid segment is located at the 5′ end of the sequencing primer and is configured to specifically bind to at least a portion of a sequence within a second region of the target nucleic acid molecule.

In the sequencing primer provided in examples of the present application, the number of the first nucleic acid segments is one or multiple, and the number of second nucleic acid segments may also be one or multiple. In some embodiments, in each sequencing primer, the molar ratio of the first nucleic acid segment to the second nucleic acid segment is 1:10 to 10:1. In this case, the sequencing primer may bind to one or multiple first regions and/or one or multiple second regions of the target nucleic acid molecule at the same time. When the sequencing primer comprises multiple first nucleic acid segments, the multiple first nucleic acid segments are bound to the first regions of the target nucleic acid molecule, respectively, and the binding strength between the sequencing primer and the target nucleic acid molecule is overall improved by the binding of one or multiple the second nucleic acid segments to the first region(s) of the target nucleic acid molecule. In some examples, in each sequencing primer, the number of the first nucleic acid segment and the number of the second nucleic acid segment are both not more than 5, so as to reduce the spatial hindrance to the binding of the first nucleic acid segment and the second nucleic acid segment to the target nucleic acid molecule. Illustratively, the ratio of the number of the first nucleic acid segment to the second nucleic acid segment is 1:1, 1:2, 1:3, 1:4, 1:5, 2:1, 2:2, 2:3, 2:4, 2:5, 3:1, 3:2, 3:3, 3:4, 3:5, 4:1, 4:2, 5:3, 4:4, 4:5, 5:1, 5:2, 5:3, 5:4, 5:5, etc.

In examples of the present application, from the 5′ end to the 3′ end, the sequence of the first nucleic acid segment and the sequence of second nucleic acid segment may be the same or different. In one example, from the 5′ end to the 3′ end, the sequence of the first nucleic acid segment and the sequence of second nucleic acid segment are the same. In another examples, from the 5′ end to the 3′ end, the sequence of the first nucleic acid segment and the sequence of second nucleic acid segment are complementary to each other. In a further example, from the 5′ end to the 3′ end, the sequence of the first nucleic acid segment and the sequence of second nucleic acid segment are different, and are not complementary to each other.
(3) A linker.

In examples of the present application, the linker mainly plays a role of connecting and bridging to achieve spatial spanning. In one implementation, the linker is bound to the 5′ end of the first nucleic acid segment and the 3′ end of the second nucleic acid segment, respectively. In some embodiments, the linker is bound to the 5′ end of the first nucleic acid segment through a first linking group, and is bound to the 3′ end of the second nucleic acid segment through a second linking group. In another implementation, the linker is bound to the 5′ end of the first nucleic acid segment and the 5′ end of the second nucleic acid segment, respectively, and the second nucleic acid segment has a 3′ blocked end. The “3′ occluded end” referred to in examples of the present application means that the 3′ end nucleotide of a nucleic acid molecule, in particular the 3′-OH of the five-carbon sugar on the 3′ end nucleotide, is occluded, resulting in the loss of the 3′ end that otherwise has activity to undergo a polymerization reaction with a nucleotide or an analog thereof. The 3′ occluded end can be achieved in various ways. For example, a blocking group that does not undergo a polymerization reaction with a nucleotides or an analog thereof, and particularly does not undergo a polymerization reaction with a phosphoric acid of the nucleotide or the analog thereof, is formed at the 3′ end, including but not limited to 3′ amino blocking, or using a dideoxynucleotide or an analogs thereof as the terminal nucleotide of the second nucleic acid segment, etc. In some embodiments, the linker is bound to the 5′ end of the first nucleic acid segment through a first linking group, and is bound to the 3′ end of the second nucleic acid segment through a second linking group.

In examples of the present application, one or multiple first linking groups are present in the linker. When multiple first linking groups are present in the linker, the multiple first linking groups may be bound to the 5′ end of one first nucleic acid segment, or may be bound to the 5′ ends of multiple first nucleic acid segments.

In examples of the present application, one or multiple second linking groups are present in the linker. When multiple second linking groups are present in the linker, the multiple second linking groups may be bound to the 3′ end or 5′ end of one second nucleic acid segment, or may be bound to the 3′ ends or 5′ ends of multiple second nucleic acid segments.

Each sequencing primer provided in examples of the present application comprises N first nucleic acid segments, and the N first nucleic acid segments are linked by means of 1 to N linkers, N being a positive integer less than or equal to 10. Similarly, each sequencing primer may comprise M second nucleic acid segments, and the M second nucleic acid segments are linked by means of 1 to M linkers, M being a positive integer less than or equal to 10. The above two configurations may exist in the same example.

In one embodiment, each sequencing primer comprises N first nucleic acid segments, and the N first nucleic acid segments are linked by means of one linker, N being a positive integer less than or equal to 10; each sequencing primer comprises M second nucleic acid segments, and the M second nucleic acid segments are linked by means of one linker, M being a positive integer less than or equal to 10. In this case, N first linking groups and M second linking groups are present in the linker, the N first nucleic acid segments are linked to the linker by means of the N first linking groups, and the M second nucleic acid segments are linked to the linker by means of the M second linking groups. In some examples, M≤N; that is, the number of second nucleic acid segments is less than or equal to the number of first nucleic acid segments in the sequencing primer. Illustratively, M=N=1; that is: the sequencing primer is composed of one first nucleic acid segment, one linker and one second nucleic acid segment, and the first nucleic acid segment and the second nucleic acid segment are located at two ends of the linker, respectively.

In some embodiments, each sequencing primer comprises N first nucleic acid segments, and the N first nucleic acid segments are linked to the linker by means of N first linking groups, respectively, N being a positive integer less than or equal to 10; and each sequencing primer comprises M second nucleic acid segments, and the M second nucleic acid segments are linked to the linker by means of M second linking groups, respectively, M being a positive integer less than or equal to 10. The N first linking groups are derived from one linker, and the M second nucleic acid segments are derived from one linker. In some examples, M & N; that is, the number of second nucleic acid segments is less than or equal to the number of first nucleic acid segments in the sequencing primer. For example, M=N=1; that is: the sequencing primer is composed of one first nucleic acid segment, one linker and one second nucleic acid segment, and the first nucleic acid segment and the second nucleic acid segment are located at two ends of the linker, respectively.

In some embodiments, each sequencing primer comprises N first nucleic acid segments, and the N first nucleic acid segments are linked to one linker by means of N first linking groups, respectively, N being a positive integer less than or equal to 10; each sequencing primer comprises M second nucleic acid segments, and the M second nucleic acid segments are sequentially linked by means of M linkers, respectively, M being a positive integer less than or equal to 10, and the first nucleic acid segments are bound to the linker near the 3′ end of the sequencing primer. In some examples, M≤N. Illustratively, M=N=1.

In some preferred embodiments, each sequencing primer comprises one first nucleic acid segment; each sequencing primer comprises M second nucleic acid segments, and the M second nucleic acid segments are sequentially linked by means of M linkers, respectively, M being a positive integer less than or equal to 10, and the first nucleic acid segments are bound to the linker near the 3′ end of the sequencing primer.

In some preferred embodiments, each sequencing primer comprises N first nucleic acid segments, and the N first nucleic acid segments are linked to the linker by means of N first linking groups, respectively, N being a positive integer less than or equal to 10; each sequencing primer comprises M second nucleic acid segments, and the M number of the second nucleic acid segments are linked by means of M linkers, respectively, M being a positive integer less than or equal to 10.

When the two ends of a linker comprises one first linker group and one second linker group, respectively, the structure of the linker is overall a linear structure. When a linker comprises multiple first linking groups and/or second linking groups at the same time, and the linker links multiple first nucleic acid segments and/or second nucleic acid segments by means of the first linking groups and/or second linking groups, the linker may form a star structure. The multiple first linking groups and/or second linking groups may be located at the ends of the linker, or may be provided in the structure of the linker in a radial construction.

In some embodiments, the first linking group and/or the second linking group are each independently selected from a hydroxyl group, a carboxyl group, a sulfonate group, an ester group, a phosphate ester group, an amino group, an ether group, a saccharide group, an alkyl group, an amide group, and an aromatic ring. The first linking group and the second linking group may also be each independently selected from a biological macromolecule having a molecular weight of not more than 100,000. In this case, the linker can link multiple first nucleic acid segments and/or second nucleic acid segments by means of the biological macromolecule at the same time, such that the two nucleic acid segments are formed on the structure of one biological macromolecule, which is beneficial to the binding of the two nucleic acid segments to a target region. Illustratively, the biological macromolecule may be a polysaccharide, a polypeptide chain, an oligomeric nucleic acid, etc.

In some embodiments, the linker has a length of 0.3 nm to 100 nm, to enable spanning over a certain sequence length or spatial distance. Illustratively, when the bound target nucleic acid molecules are nucleic acid molecules obtained by bridge amplification, the nucleic acid molecules at the two ends of the linker are bound to sequences, such as amplification primers (a complementary sequence of P5 or a complementary sequence of P7 and a sequencing primer site), at the two ends of an Index sequence, respectively. In this case, the length of the linker is greater than the length of the Index sequence. When the target nucleic acid molecules are a cluster of nucleic acid molecules comprising two inserted fragments, the nucleic acid molecules at the two ends of the linker can at least bind to the primer sites of adjacent target nucleic acid molecule clones, respectively. In this case, the length of the linker is greater than the distance between the adjacent nucleic acid molecule clones.

In examples of the present application, the type of the linker may be selected from a variety of options, and is not strictly limited. In one embodiment, the linker comprises one or multiple hydrophilic groups. Hydrophilic groups include, but are not limited to, a hydroxyl group, a carboxyl group, a sulfonate group, an ester group, a phosphate ester group, an amino group, a saccharide group, an ether group, and an amide group.

In some examples, the linker does not comprise a linear conjugated group such as a conjugated olefin, such that the linker has a better flexibility to achieve space spanning between the first region and the second region.

In some embodiments, the linker comprises at least one selected from an optionally substituted or unsubstituted C5-C20 alkylene, a C5-C20 alkylene in which one or more carbon atoms are substituted by a heteroatom, a small molecule compound, a polymer, a peptide chain, a polynucleotide, a polysaccharide, and an aromatic ring.

In some specific embodiments of the present invention, the linker is selected from a polymer, the number of backbone atoms in the linker being greater than or equal to 20 atoms, and a molecular weight thereof being greater than or equal to 2,000. In some examples, the polymer comprises at least one of a hydroxyl group, a carboxyl group, a sulfonate group, an ester group, a phosphate group, an amino group, an ether group, a saccharide group, an alkyl group, and an amide group. In one aspect, these groups have good hydrophilicity, and in another aspect, these groups can impart good flexibility to the linker, so as to achieve spanning over a nucleic acid sequence and/or space.

In examples of the present application, polymers that can be used as the linker include, but are not limited to, at least one of polyethylene glycol, polyvinyl alcohol, polyacrylamide, polymethacrylic acid, polyvinyl sulfonic acid, and polyvinylpyrrolidone. Illustratively, the polymer is at least one of iSp18 (polyethylene glycol 6), polyethylene glycol 200, polyethylene glycol 800, polyethylene glycol 2000, and polyethylene glycol 4000.

In some embodiments, the linker is selected from a small molecule compound the number of backbone atoms in which is greater than or equal to 6, and a molecular weight of which is no greater than 1,000.

In some embodiments, the linker is selected from a nucleotide or polynucleotide composed of 1 to 40 bases, preferably a polynucleotide composed of 3 to 26 bases.

The sequencing primer provided in examples of the present application is used to bind to a target nucleic acid molecule, and serves as a primer for generating a complementary strand of the target nucleic acid molecule. The target nucleic acid molecule comprises an inserted nucleic acid fragment. In some examples, the inserted nucleic acid fragment is a nucleic acid fragment having an unknown sequence.

In one embodiment, the target nucleic acid molecule comprises an inserted nucleic acid fragment, and further comprises a first region and a second region located at the 3′ end of the inserted nucleic acid fragment, and a spacer sequence is provided between the first region and the second region. The first nucleic acid segment and the second nucleic acid segment of the sequencing primer are partially complementary or completely complementary to the first region and the second region, respectively. When the first nucleic acid segment and the second nucleic acid segment of the sequencing primer are partially complementary to the first region and the second region, respectively, the complementary region accounts for 80% or more of the first region or the second region, such that the first nucleic acid segment is configured to specifically bind to the first region of the sequencing library molecule, and the second nucleic acid segment is configured to specifically bind to the second region of the sequencing library molecule.

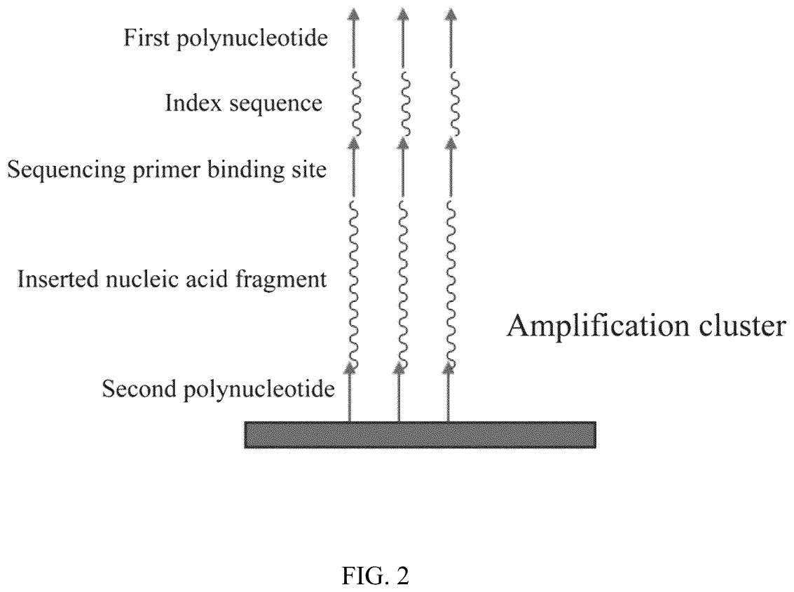

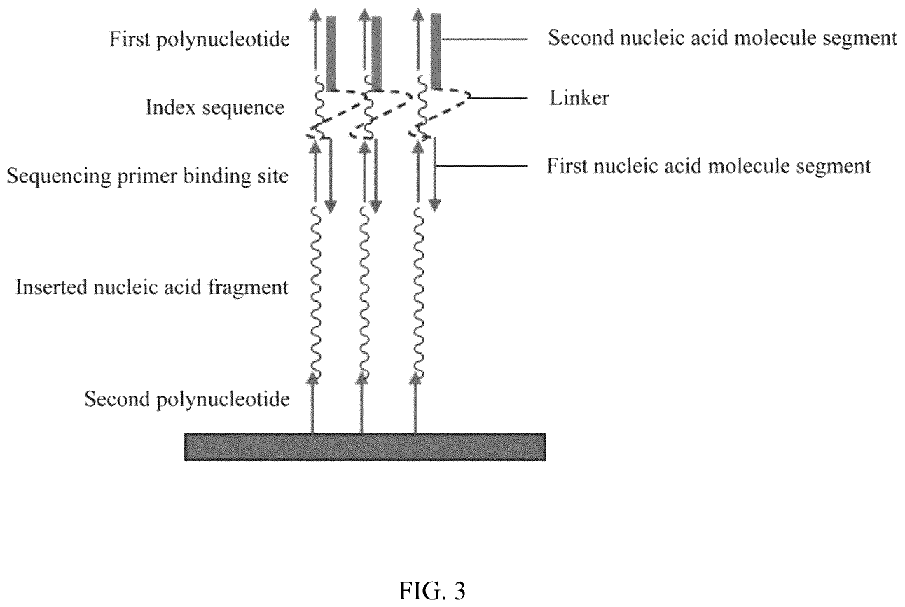

In one example, the structure of the target nucleic acid molecule is shown in FIG. 2, which comprises, sequentially from the 3′ end to the 5′ end, a first polynucleotide, an Index, a sequencing primer binding site, an inserted sequence and a second polynucleotide. In this case, the Index is an spacer sequence. As shown in FIG. 3, the linker of the sequencing primer spans over the spacer sequence, and the first nucleic acid segment and the second nucleic acid segment are bound to the sequencing primer binding site and the first polynucleotide, respectively, thereby improving the stability of binding of the first nucleic acid segment to the target nucleic acid molecule. In some examples, the target nucleic acid molecule is a nucleic acid molecule obtained by amplification, and the first polynucleotide and the second polynucleotide are amplification primers. Illustratively, for example, the first polynucleotide and the second polynucleotide are a primer set formed of a complementary sequence of P7 and P5, or a primer set formed of a complementary sequence of P5 and P7.

The length of the spacer sequence is related to the configuration of the target nucleic acid molecule, and in some examples, the length of the spacer sequence is 3 nt to 26 nt.

In some examples, the number of first nucleic acid segments is greater than or equal to the number of second nucleic acid segments. Hence, with a smaller number of the second nucleic acid segments, the binding ability of a larger number of first second nucleic acid segments to the target nucleic acid molecule may be increased. In one example, each sequencing primer comprises X first nucleic acid segments, and the X first nucleic acid segments specifically bind to the first regions of X target nucleic acid molecules, respectively, X being a positive integer less than or equal to 10. Illustratively, X is selected from 1, 2, 3, 4, 5, 6, 7, 8, etc. In one example, the number of first nucleic acid segments is equal to the number of second nucleic acid segments. Illustratively, in each sequencing primer, the number of first nucleic acid segments and the number of second nucleic acid segments are both 1.

The sequencing primer (tripartite primer) constructed in this embodiment is designed as two nucleic acid segments linked by means of a linker, such that the sequencing primer is separately bound to sites at different positions of the same target nucleic acid molecule by means of the linker “spanning over” a spatial distance, thereby improving the structural stability of a double-stranded structure formed by the sequencing primer and the target nucleic acid molecule. Therefore, when the sequencing primer is used to perform nucleic acid sequencing, rapid sequencing can be achieved under conditions of higher temperature and higher pH value without loss of accuracy, thereby improving the sequencing speed and the accuracy of the sequencing result.

In another embodiment, two or more target nucleic acid molecule clones form a cluster of clones, and each target nucleic acid molecule in the cluster of clones comprises at least two inserted nucleic acid fragments: a first inserted nucleic acid fragment and a second inserted nucleic acid fragment. The second inserted nucleic acid fragment is located at the 5′ end of the first inserted nucleic acid fragment. The target nucleic acid molecule further comprises primer binding sites used for performing sequencing of the first inserted nucleic acid fragment and the second inserted nucleic acid fragment, respectively. Specifically, a first primer binding site is provided at the 3′ end of the first inserted nucleic acid fragment, and is used to provide a primer binding site for sequencing the first inserted nucleic acid fragment; a second primer binding site is provided at the 3′ end of the second inserted nucleic acid fragment, and is used to provide a primer binding site for sequencing the second inserted nucleic acid fragment. Since, in the cluster of clones, the first inserted nucleic acid fragment and the second inserted nucleic acid fragment are in the same amount or molar ratio, when the first inserted nucleic acid fragment and the second inserted nucleic acid fragment are synchronously sequenced, the sequencing signals generated by the two fragments cannot be distinguished. The inventors have found that the sequencing primer provided by examples of the present application can cause the sequencing signals generated by the first inserted nucleic acid fragment and the second inserted nucleic acid fragment to be identifiably different when the two fragments are synchronously sequenced. For example, the first nucleic acid segment and the second nucleic acid segment bind to the first primer binding sites of different target nucleic acid molecules in the cluster of clones. That is, the first region and the second region in the target nucleic acid molecule are the first primer binding sites of different target nucleic acid molecules in the cluster of clones. In this case, since the 3′ end of some of the second nucleic acid segments does not have an extension activity, the first primer binding sites of some of the target nucleic acid molecules in the cluster of clones are blocked, such that the number of sequencing primers (i.e., first nucleic acid segments) bound to the first primer binding sites is less than the number of sequencing primers bound to the second primer binding sites. Thus, when the first inserted nucleic acid fragment and the second inserted nucleic acid fragment are synchronously extended, the sequencing signals generated by the two fragments are identifiably different, thereby achieving synchronous sequencing of two regions to be determined of the same target nucleic acid molecule.

In some examples, from the 5′ end to the 3′ end or from the 3′ end to the 5′ end, the sequence of the first nucleic acid segment is identical or complementary to the sequence of the second nucleic acid segment. In some examples, a second primer binding site is provided between the first inserted nucleic acid fragment and the second inserted nucleic acid fragment.

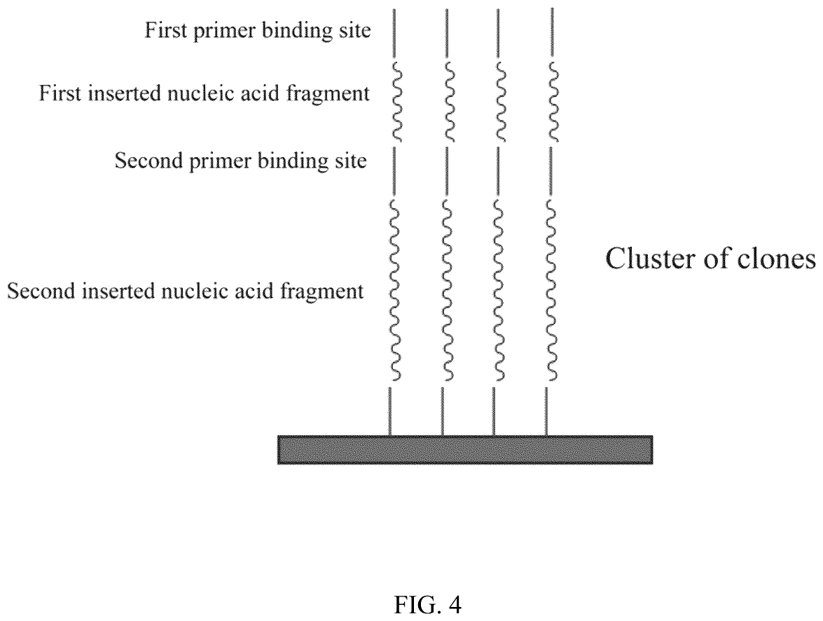

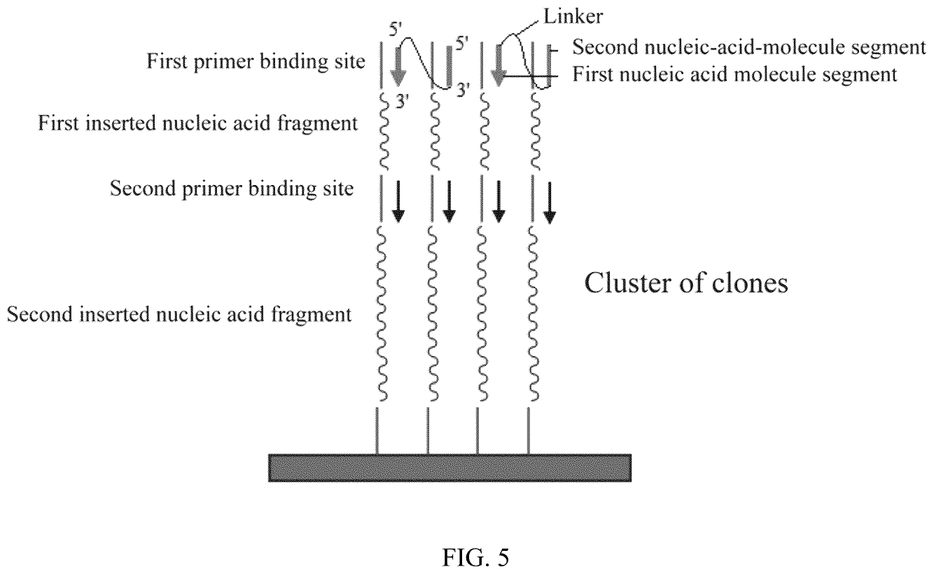

In one example, the structure of target nucleic acid molecules in a cluster of clones is shown in FIG. 4, which comprises, from the 3′ end to the 5′ end, a first primer binding site, a first inserted nucleic acid fragment, a second primer binding site, and a second inserted nucleic acid fragment. In one embodiment, the linker is bound to the 5′ end of the first nucleic acid segment and the 3′ end of the second nucleic acid segment, respectively. In this case, after binding with the sequencing primer provided in examples of the present application, as shown in FIG. 5: the first nucleic acid segment and the second nucleic acid segment are bound to the first primer binding sites of different target nucleic acid molecules in the cluster of clones, respectively. When a nucleotide or an analog thereof is used to extend the primer strand, the 3′ end of the first nucleic acid segment bound to the first primer binding site has a reactive group and is capable of undergoing a polymerization reaction with the nucleotide or the analog thereof. By contrast, due to being linked to the linker, the 3′ end of the second nucleic acid segment bound to the first primer binding sites loses the activity of undergoing a polymerization reaction with the nucleotide or the analog thereof, thus resulting in the first primer binding sites of some of the nucleic acid clones being blocked. When the second primer binding sites are not blocked, the number of the primer that binds to the first primer binding sites and can be extended is smaller than the number of the primer that binds to the second primer binding sites and can be extended. Therefore, the sequencing signals generated by sequencing the first inserted nucleic acid fragment and the sequencing signals generated by sequencing the second inserted nucleic acid fragment become different. Since the difference between the two fragments is related to the number of primer strands extendable for the two fragments, and the difference is basically stable during the sequencing process, synchronous sequencing of the two inserted nucleic acid fragments can be achieved by qualitatively determining the difference between the two fragments.

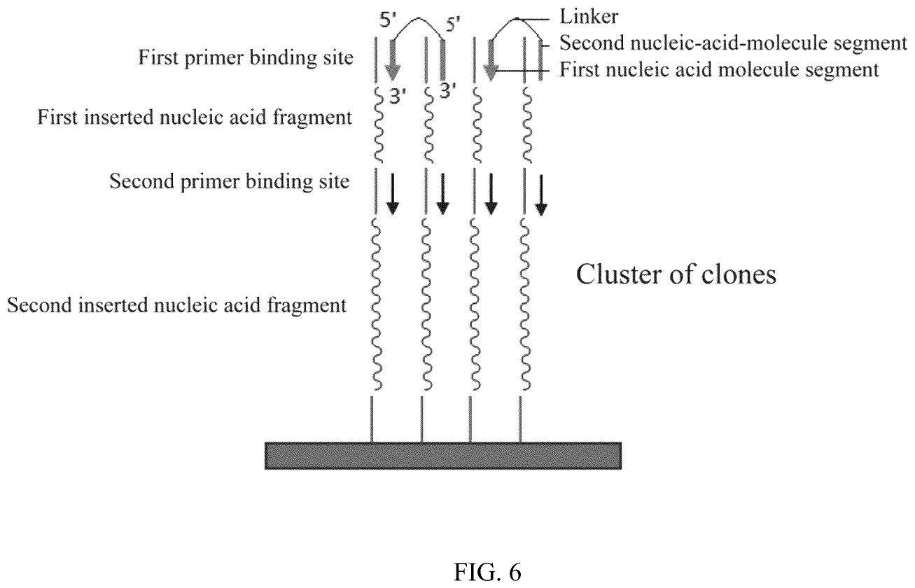

In another embodiment, the linker is bound to the 5′ end of the first nucleic acid segment and the 5′ end of the second nucleic acid segment, respectively, and the second nucleic acid segment has a 3′ occluded end. In this case, after binding with the sequencing primer provided in examples of the present application, as shown in FIG. 6: the first nucleic acid segment and the second nucleic acid segment are bound to the first primer binding sites of different target nucleic acid molecules in the cluster of clones. When a nucleotide or an analog thereof is used to extend the primer strand, the 3′ end of the first nucleic acid segment bound to the first primer binding site has a reactive group, and is capable of undergoing a polymerization reaction with the nucleotide or the analog thereof. By contrast, due to being blocked, the 3′ end of the second nucleic acid segment bound to the first primer binding site loses the activity of undergoing a polymerization reaction with the nucleotide or the analog thereof, thus resulting in the first primer binding sites of some of the nucleic acid clones being blocked. When the second primer binding sites are not blocked, the number of the primer that binds to the first primer binding sites and can be extended is smaller than the number of the primer that binds to the second primer binding sites and can be extended. Therefore, the sequencing signals generated by sequencing the first inserted nucleic acid fragments and the sequencing signals generated by sequencing the second inserted nucleic acid fragments become different. Since the difference between the two fragments is related to the number of primer strands extendable for the two fragments, and the difference is basically stable during the sequencing process, the two inserted nucleic acid fragments can be synchronously sequenced by qualitatively determining the difference between the two fragments.

In some examples, a first polynucleotide sequence is provided at the 3′ end of the first primer binding site. In some examples, a second polynucleotide sequence is provided at the 5′ end of the second inserted nucleic acid fragment. Illustratively, the first polynucleotide sequence and the second polynucleotide sequence are each independently an amplification primer, and the two constitute an amplification primer set, such as a primer set formed of a complementary sequence of P7 and P5, or a primer set formed of a complementary sequence of P5 and P7.

In examples of the present application, the number of the first nucleic acid segment may be greater than, equal to, or less than the number of the second nucleic acid segment in the sequencing primer. In some examples in which the number of first nucleic acid segments is greater than or less than the number of second nucleic acid segments in the sequencing primer, the number of first nucleic acid segments differs from the number of second nucleic acid segments by greater than or equal to 20%, such that the difference between the sequencing signals obtained by sequencing the first inserted nucleic acid fragment and the sequencing signals obtained by sequencing the second inserted nucleic acid fragment is more easily identified. In one example, the number of first nucleic acid segments is less than or equal to the number of second nucleic acid segments in the sequencing primer, and the molar ratio of the first nucleic acid segment to the second nucleic acid segment is 1:1 to 1:5. Illustratively, the molar ratio of the first nucleic acid segment to the second nucleic acid segment is 1:1, 1:2, 1:3, 1:4, 1:5, etc.

The sequencing primer (tripartite primer) constructed in this embodiment is designed as two nucleic acid segments linked by means of a linker, such that the sequencing primer is separately bound to sites at the same position of adjacent target nucleic acid molecules in the cluster of clones by means of the linker “spanning over” a spatial distance, such that when target nucleic acid molecules comprising two inserted nucleic acid fragments are sequenced, synchronous sequencing of the two inserted nucleic acid fragments can be achieved, thereby increasing sequencing throughput. On the basis of the two different types of target nucleic acid molecule described above, examples of the present application provide two methods for performing sequencing by using the foregoing sequencing primer.

A first sequencing method comprises the following steps:
(a1) Hybridizing the sequencing primer with a nucleic acid template, to form a primer-nucleic acid template complex.

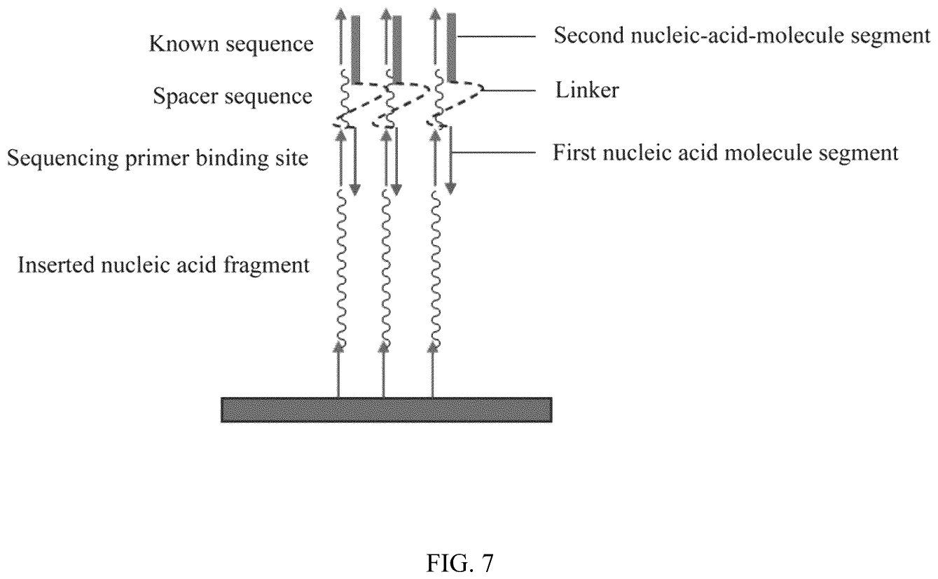

In this step, referring to FIG. 7, the nucleic acid template comprises an inserted nucleic acid fragment, the inserted nucleic acid fragment being a sequence to be determined having an unknown sequence. A sequencing primer binding site and a known sequence are located at the 3′ end of the inserted nucleic acid fragment, and the known sequence is located at the 3′ end of the sequencing primer binding site. It is to be understood that the sequencing primer binding site also has a known sequence, but the sequence of the sequencing primer binding site is different from that of the known sequence located at the 3′ end of the sequencing primer binding site. In some examples, the known sequence is a first adaptor. In some embodiments, the first adaptor serves as an amplification primer for amplification of the inserted nucleic acid fragment. Illustratively, the first adaptor is a complementary sequence of P5 or a complementary sequence of P7, and is used for hybridizing with P5 or P7 to achieve amplification of a nucleic acid molecule comprising the inserted nucleic acid fragment, such as bridge amplification. In some examples, a polynucleotide sequence is provided at the 5′ end of the inserted nucleic acid fragment, and is used for fixing or linking the inserted nucleic acid fragment. In some examples, the polynucleotide sequence at the 5′ end of the inserted nucleic acid fragment is a second adaptor. In some embodiments, the second adaptor serves as an amplification primer for amplification of the inserted nucleic acid fragment. Illustratively, the second adaptor is P5 or P7, and is used as an amplification primer to achieve amplification of a nucleic acid molecule comprising the inserted nucleic acid fragment, such as bridge amplification. In one example, the combination of the polynucleotide sequence at the 5′ end of the inserted nucleic acid fragment and the known sequence is a combination formed of a complementary sequence of P7 and P5, or a combination formed of a complementary sequence of P5 and P7.

In examples of the present application, a spacer sequence is present between the sequencing primer binding site and the known sequence. In some examples, the spacer sequence comprises a tag sequence, i.e., an Index sequence. In one example, the spacer sequence is a tag sequence. In this example, the length of the spacer sequence is 3 nt to 26 nt. In one example, referring to FIG. 2 and FIG. 3, from the 3′ end to the 5′ end, the nucleic acid template sequentially comprises a first polynucleotide, an Index sequence, a sequencing primer binding site, an inserted nucleic acid fragment, and a second polynucleotide sequence, wherein the combination of the first polynucleotide sequence and the second polynucleotide sequence is a combination formed of a complementary sequence of P7 and P5, or a combination formed of a complementary sequence of P5 and P7.

In some examples, the nucleic acid template provided in examples of the present application is fixed on a surface of a solid phase substrate by means of the 5′ end, and the method of fixing is not strictly limited. The nucleic acid template fixed on the surface of the solid phase substrate may be a single nucleic acid molecule formed on the surface of the solid phase substrate, or may be a cluster of clones formed of nucleic acid molecules. In one implementation, the cluster of clones may be formed by means such as bridge amplification. In one example, a cluster of clones formed by the nucleic acid template is provided on the solid phase substrate, and the nucleic acid template at least comprises, from the 3′ end to the 5′ end: a known sequence, a spacer sequence, a sequencing primer binding site, and an inserted nucleic acid fragment.

In this step, the sequencing primer is the sequencing primer (tripartite primer) described above, comprising the linker, the first nucleic acid segment and the second nucleic acid segment. The first nucleic acid segment is located at the 3′ end of the sequencing primer, and the 3′ end of the first nucleic acid segment has a reactive group. In examples of the present application, the reactive group at the 3′ end of the first nucleic acid segment has reactivity to undergo a polymerization reaction with a nucleotide or an analog thereof, such that the first nucleic acid segment can be extended at the 3′ end by means of the polymerization reaction. In some embodiments, the reactive group comprises —OH. In this case, the —OH at the 3′ end of the first nucleic acid segment reacts with a phosphate group of the nucleotide or the analog thereof to form a phosphate ester group, thereby achieving the extension of the nucleotide or the analog thereof. As part of the sequencing primer, the first nucleic acid segment is configured to be partially complementary or completely complementary to a sequence of the sequencing primer binding site of the nucleic acid template, and can undergo a polymerization reaction with the nucleotide or the analog thereof by means of the reactive group at the 3′ end to achieve extension of the primer strand from the 5′ end to the 3′ end. In one example, the first nucleic acid segment is configured to be completely complementary to a sequence of the sequencing primer binding site of the nucleic acid template.

The second nucleic acid segment is located at the 5′ end of the sequencing primer, and is configured to be partially complementary or completely complementary to a sequence of the known sequence of the target nucleic acid molecule. In one example, the second nucleic acid segment is configured to be completely complementary to a sequence of the known sequence of the target nucleic acid molecule.

Since the sequence composition of the sequencing primer binding site is generally different from that of the known sequence, the sequence of the first nucleic acid segment is different from that of the second nucleic acid segment in the sequencing primer.

In some examples, the nucleic acid template is a cluster of clones formed of nucleic acid molecules. In each sequencing primer, the number of the first nucleic acid segment is greater than or equal to the number of the second nucleic acid segment. In this case, the binding firmness between the first nucleic acid segment in a greater number and the sequencing primer binding site is improved by means of the second nucleic acid segment in a fewer number, thereby improving the binding stability between the sequencing primer as a whole and the nucleic acid template. Illustratively, in each sequencing primer, the number of first nucleic acid segments is 2, and the number of second nucleic acid segments is 1 or 2; in each sequencing primer, the number of first nucleic acid segments is 3, and the number of second nucleic acid segments is 1, 2 or 3; and in each sequencing primer, the number of first nucleic acid segments is 4, and the number of second nucleic acid segments is 1, 2, 3 or 4. In one example, the number of first nucleic acid segments is equal to the number of second nucleic acid segments in the sequencing primer. Illustratively, in each sequencing primer, the number of first nucleic acid segments and the number of second nucleic acid segments are both 1.

In this example, the linker is used to link the first nucleic acid segment used as a sequencing primer and the second nucleic acid segment used for enhancing the stability of primer linking, so as to span a spatial region, i.e., the spacer sequence, between the sequences bound by the two nucleic acid segments. Specifically, the linker is bound to the 5′ end of the first nucleic acid segment and the 3′ end of the second nucleic acid segment, respectively.

In some embodiments, the linker has a length of 0.3 nm to 100 nm, to enable spanning over a certain sequence length. Illustratively, the length of the linker may be in the range of 0.3 nm to 1 nm, 0.3 nm to 5 nm, 0.3 nm to 10 nm, 1 nm to 10 nm, 1 nm to 20 nm, 5 nm to 20 nm, 10 nm to 20 nm, 10 nm to 50 nm, 20 nm to 50 nm, 30 nm to 50 nm, 50 nm to 100 nm, 50 nm to 60 nm, 50 nm to 80 nm, etc. In examples of the present application, the type of the linker may be selected from a variety of options, and is not strictly limited. In one embodiment, the linker comprises one or multiple hydrophilic groups. The hydrophilic group comprises, but is not limited to, a hydroxyl group, a carboxyl group, a sulfonate group, an ester group, a phosphate ester group, an amino group, an ether group, and an amide group.

In some embodiments, the linker comprises at least one selected from an optionally substituted or unsubstituted C5-C20 alkylene, a C5-C20 alkylene in which one or more carbon atoms are substituted by a heteroatom, a small molecule compound, a polymer, a peptide chain, a polynucleotide, a polysaccharide, and an aromatic ring.

In some specific embodiments of the present invention, the linker is selected from a polymer, the number of backbone atoms in the polymer being greater than or equal to 300 atoms, and a molecular weight thereof being greater than or equal to 4,000. In some examples, the polymer comprises at least one of a hydroxyl group, a carboxyl group, a sulfonic acid group, an ester group, a phosphate group, an amino group, an ether group, and an amide group. In one aspect, these groups have good hydrophilicity; in another aspect, these groups can impart good flexibility to the linker, to achieve spanning over a nucleic acid sequence and/or space.

In examples of the present application, polymers that can be used as the linker include, but are not limited to, at least one of polyethylene glycol, polyvinyl alcohol, polyacrylamide, polymethacrylic acid, polyvinyl sulfonic acid, and polyvinylpyrrolidone. Illustratively, the polymer is at least one of iSp18 (polyethylene glycol 6), polyethylene glycol 200, polyethylene glycol 800, polyethylene glycol 2000, and polyethylene glycol 4000.

In some embodiments, the linker is selected from a small molecule compound the number of backbone atoms in which is greater than or equal to 6 and a molecular weight of which is no greater than 1,000.

In some specific embodiments of the present invention, the linker is a polynucleotide, and the length of the polynucleotide is greater than the length of the spacer sequence. Illustratively, when the spacer sequence is an Index sequence, the length of the linker is greater than the length of the Index sequence. In some examples, the GC content in the polynucleotide is 0% to 70%, which can reduce the influence of excessive hydrogen bonds on the flexibility of the linker. Of course, an excessively high GC content, such as a GC content of more than 70%, provides a favorable condition for the formation of a G-quadruplex, and a large number of G-quadruplex structures will appear in the polynucleotide structure, converting the linker into a more rigid structure.

In some embodiments, the linker is selected from a nucleotide or polynucleotide composed of 1 to 40 bases, preferably a polynucleotide composed of 3 to 26 bases.

In some embodiments, the linker is bound to the 5′ end of the first nucleic acid segment through a first linking group, and is bound to the 3′ end of the second nucleic acid segment through a second linking group. In examples of the present application, one or multiple first linking groups are present in the linker. When multiple first linking groups are present in the linker, the multiple first linking groups may be bound to the 5′ end of one first nucleic acid segment, or may be bound to the 5′ ends of multiple first nucleic acid segments. The manner in which the first linking group may be bound to the 5′ end of the first nucleic acid segment includes chemical bonding, hydrogen bond formation, etc. Illustratively, the first linking group is selected from a hydroxyl group, a carboxyl group, a sulfonate group, an ester group, a phosphate ester group, an amino group, an ether group, a saccharide group, an alkyl group, and an amide group.

In examples of the present application, one or multiple second linking groups are present in the linker. When multiple second linking groups are present in the linker, the multiple second linking groups may be bound to the 3′ end of one second nucleic acid segment, or may be bound to the 3′ ends of a multiple number of the second nucleic acid segments. The manner in which the second linking group may be bound to the 5′ end of the second nucleic acid segment includes chemical bonding, hydrogen bond formation, etc. Illustratively, the second linking group is selected from a hydroxyl group, a carboxyl group, a sulfonate group, an ester group, a phosphate ester group, an amino group, an ether group, a saccharide group, an alkyl group, an amide group, and aromatic ring, or a biological macromolecule having a molecular weight of not more than 100,000. Illustratively, the biological macromolecule is selected from a polysaccharide, a polypeptide chain, and an oligomeric nucleic acid.

In examples of the present application, one sequencing primer may only comprise one first nucleic acid segment, and the 5′ end of the first nucleic acid segment is linked to the linker. One sequencing primer may also comprise two or more first nucleic acid segments, and the 5′ ends of the multiple number of the first nucleic acid segments are linked to the linker by means of multiple first linking groups on the same linker.

In examples of the present application, one sequencing primer may only comprise one second nucleic acid segment, and the 3′ end of the second nucleic acid segment is linked to the linker. One sequencing primer may also comprise two or more second nucleic acid segments, and the 3′ ends of the multiple number of the second nucleic acid segments are linked to the linker via a multiple number of second linking groups on the same linker.

Of course, one sequencing primer may comprise one or multiple first nucleic acid segments, and at the same time comprise one or multiple second nucleic acid segments. Illustratively, one sequencing primer comprises one first nucleic acid segment and one second nucleic acid segment; or, one sequencing primer comprises two or more first nucleic acid segments and one second nucleic acid segment; or, one sequencing primer comprises one first nucleic acid segment and two or more second nucleic acid segments; or, one sequencing primer comprises two or more first nucleic acid segments and two or more second nucleic acid segments at the same time.

In some examples, when the sequencing primer comprises multiple first nucleic acid segments and/or multiple second nucleic acid segments, the multiple first nucleic acid segments are linked by means of the same linker, and the multiple second nucleic acid segments are linked by means of the same linker. In some examples, when the sequencing primer comprises multiple first nucleic acid segments and multiple second nucleic acid segments, the multiple first nucleic acid segments are linked to the second nucleic acid segments by means of multiple linkers, respectively, and each of the multiple linkers is linked to at least one of the other linkers.

In this step, the sequencing primer is contacted with the nucleic acid template, the first nucleic acid segment is partially complementary or completely complementary to the sequencing primer binding site, and the second nucleic acid segment is partially complementary or completely complementary to the known sequence of the nucleic acid template, such that hybridization of the sequencing primer with the nucleic acid template is achieved, and a primer-nucleic acid template complex is obtained. The primer-nucleic acid template complex thus obtained has improved structural stability, and hence, a chemical reaction under harsher conditions can be achieved. For example, rapid sequencing can be achieved under conditions of higher temperature and higher pH value without loss of accuracy, thereby improving sequencing speed and the accuracy of the sequencing result.

In one example, each sequencing primer comprises X first nucleic acid segments, and the X first nucleic acid segments are partially complementary or completely complementary to the sequencing primer binding sites of X nucleic acid templates, respectively, X being a positive integer greater than 1 and less than 10. Illustratively, X is selected from 1, 2, 3, 4, 5, 6, 7, 8, etc. In one example, the number of first nucleic acid segments is equal to the number of second nucleic acid segments. Illustratively, in each sequencing primer, the number of first nucleic acid segments and the number of second nucleic acid segments are both 1.
(b1) Contacting one of the following substrates or substrate combinations i) to iii) and a polymerase with the primer-nucleic acid template complex, such that the substrate or substrate combination is bound to the polymerase and the primer-nucleic acid template complex and/or the substrate or substrate combination is incorporated into the 3′ end of the primer strand of the primer-nucleic acid template complex,
i) a first nucleotide, the first nucleotide comprising a fluorescent label, and a blocking group used to prevent the incorporation of a next nucleotide or an analog thereof into the primer strand,
ii) a second nucleotide, the second nucleotide being a nucleotide containing a fluorescent label or an analog thereof, and
iii) a third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand.

In this step, the first nucleotide, the second nucleotide and the fluorescently labeled antibody comprise a fluorescent label, the first nucleotide polymerizes with the primer-nucleic acid template complex under the action of the polymerase, and the second nucleotide and the fluorescently labeled antibody can bind to the primer-nucleic acid template complex. The type of the nucleotide incorporated upon primer strand extension can be determined by means of determining the fluorescent label in the fluorescently labeled antibody or nucleotide that is bound to or polymerized with the primer-nucleic acid template complex.

In one embodiment, the substrate is i), i.e., a first nucleotide, the first nucleotide comprising a fluorescent label and a blocking group used to prevent the incorporation of a next nucleotide or an analog thereof into the primer strand, and in this case, step (b1) is (b11). In step (b11), the first nucleotide, the polymerase and the primer-nucleic acid template complex are contacted under conditions suitable for a polymerization reaction to occur. The reactive group at the 3′ end of the primer strand in the primer-nucleic acid template complex, i.e., the 3′ end of the first nucleic acid segment, is polymerized with the first nucleotide, such that the first nucleotide is incorporated into the 3′ end of the primer strand of the primer-nucleic acid template complex, thereby achieving extension of the 3′ end of the primer strand. The type of the first nucleotide incorporated at the 3′ end of the primer strand of the primer-nucleic acid template complex can be determined by means of performing signal acquisition and identification on the fluorescent label of the first nucleotide. In addition, since the first nucleotide further comprises a blocking group used to prevent the incorporation of another nucleotide into the primer strand, in each instance of step (b11), the 3′ end of the primer strand tends to introduce a nucleotide reversible terminator, so as to extend one nucleotide.

In this example, nucleotide reversible terminators include reversible terminator dATP, reversible terminator dTTP, reversible terminator dGTP and reversible terminator dCTP. In step (b11), the reversible terminator dATP, reversible terminator dTTP (or reversible terminator dUTP), reversible terminator dGTP, and reversible terminator dCTP may be added at the same time; or, the four kinds of reversible terminator may be grouped into two combinations, with one combination containing two of the reversible terminators and the other combination containing the other two of the reversible terminators, or one combination containing one of the reversible terminators and the other combination containing the other three of the reversible terminators, so as to add the four kinds of reversible terminator in two instances; or, of course, the four kinds of reversible terminator may also be added separately in four instances. One instance of addition refers to a process in which a reversible terminator or reversible terminators and the polymerase are added such that one nucleotide reversible terminator is incorporated into the 3′ end of the primer strand of at least one primer-nucleic acid template complex, and signal acquisition is achieved. In one example, step (b11) and step (c1) described below are regarded as a cycle of one instance of addition. In some examples, for the sake of sequencing efficiency, in step (b11), the reversible terminator dATP, reversible terminator dTTP (or reversible terminator dUTP), reversible terminator dGTP and reversible terminator dCTP are added at the same time to perform the polymerization reaction.

In this example, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the first nucleotide and the polymerase is contacted with the primer-nucleic acid template complex, such that the first nucleotide is incorporated into the 3′ end of the primer strand of the primer-nucleic acid template complex. The solution containing the first nucleotide and the polymerase may further contain a buffer, a divalent metal ion, an ammonium ion, etc., wherein the divalent metal ion may be at least one of Mg²⁺ and/or Mn²⁺, so as to promote the occurrence of the polymerization reaction.

In step (b11), the first nucleotide, the polymerase, and the primer-nucleic acid template complex are contacted under conditions suitable for a polymerization reaction to occur, such that one first nucleotide is incorporated into the 3′ end of the primer strand of at least one primer-nucleic acid template complex under the action of the polymerase. The conditions suitable for a polymerization reaction to occur refer to conditions capable of initiating a polymerization reaction between a phosphate group of the first nucleotide and the reactive group at the 3′ end of the primer strand. In some examples, the conditions suitable for a polymerization reaction to occur include one or two of the following conditions 1) and 2):
1) A reaction temperature of 30° C. to 75° C.; and
2) A pH of 8.0 to 10.

Illustratively, the described reaction temperature may specifically be 30° C., 32° C., 35° C., 36° C., 37° C., 38° C., 39° C., 40° C., 41° C., 42° C., 43° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the described pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application.

The described reaction temperature and pH cover relatively mild reaction conditions, for example, a reaction temperature of 30° C. to 55° C., and a pH of 8.0 to 8.5. However, it is worth noting that the present application uses the described sequencing primer to bind to the nucleic acid template, which improves the stability of the primer-nucleic acid template complex. On that basis, the polymerization reaction can be carried out under higher temperature and pH conditions, and the intactness and stability of the double-stranded structure can still be maintained. In some examples, the conditions for the polymerization reaction include: a reaction condition of a pH of 9.1 to 10.0, or a reaction condition of a temperature of 60° C. to 75° C., or reaction conditions of a pH of 9.1 to 10.0 and a temperature of 60° C. to 75° C. In some examples, the conditions for the polymerization reaction include: a reaction condition of a pH of 9.8 to 10.0, or a reaction condition of a temperature of 65° C. to 75° C., or reaction conditions of a pH of 9.8 to 10.0 and a temperature of 65° C. to 75° C.

In some examples, the polymerase is Phi29 or Klenow or a mutant thereof, and the temperature suitable for the polymerization reaction to occur is 30° C. to 45° C. In some examples, the polymerase is 9° N or KOD or a mutant thereof, and the temperature suitable for the polymerization reaction to occur is 50° C. to 75° C.

In some examples, step (b11) further comprises: washing to remove the first nucleotide which has not been reacted.

In one embodiment, the substrate is ii), i.e., a second nucleotide, the second nucleotide being a nucleotide containing a fluorescent label or an analog thereof, and in this case, step (b1) is (b12). In step (b12), the second nucleotide, the polymerase and the primer-nucleic acid template complex are contacted under non-polymerization reaction conditions. Under the action of the polymerase, the second nucleotide binds to the polymerase and the primer-nucleic acid template complex, to form a structurally larger complex structure. In the complex structure, the second nucleotide does not polymerize with the primer-nucleic acid template complex. That is, under non-polymerization reaction conditions, no polymerization occurs between the reactive group at the 3′ end of the primer strand of the primer-nucleic acid template complex, i.e., the 3′ end of the first nucleic acid segment, and the second nucleotide, and the second nucleotide is not incorporated into the 3′ end of the primer strand of the primer-nucleic acid template complex. The type of a next nucleotide incorporated at the 3′ end of the primer strand of the primer-nucleic acid template complex can be determined by performing signal acquisition and identification on the fluorescent label in the second nucleotide.

In some examples, since the second nucleotide does not undergo a polymerization reaction with the primer-nucleic acid template complex, the second nucleotide may not have a blocking group used to prevent the incorporation of another nucleotide into the primer strand. In some other examples, the second nucleotide may also have a blocking group used to prevent the incorporation of another nucleotide into the primer strand.

In this example, the second nucleotide includes fluorescently labeled nucleotide dATP or an analog thereof, fluorescently labeled nucleotide dTTP or an analog thereof, fluorescently labeled nucleotide dGTP or an analog thereof, and fluorescently labeled nucleotide dCTP or an analog thereof. In step (b12), the four kinds of fluorescently labeled nucleotides may be added at the same time; or, the four kinds of fluorescently labeled nucleotides may be grouped into two combinations, with one combination containing two of the fluorescently labeled nucleotides and the other combination containing the other two of the fluorescently labeled nucleotides, or one combination containing one of the fluorescently labeled nucleotides and the other combination containing the other three of the fluorescently labeled nucleotides, so as to add the four kinds of fluorescently labeled nucleotides in two instances; or, of course, the four kinds of fluorescently labeled nucleotides may also be added separately in four instances. On instance of addition refers to a process in which a fluorescently labeled nucleotide or fluorescently labeled nucleotides and the polymerase are added such that one nucleotide reversible terminator is incorporated into the 3′ end of the primer strand of at least one primer-nucleic acid template complex, and signal acquisition is achieved. In one example, step (b12) and step (c1) described below are regarded as a cycle of one instance of addition. In some examples, for the sake of sequencing efficiency, in step (b12), the four kinds of fluorescently labeled nucleotides are added at the same time.

In this example, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the second nucleotide and the polymerase is contacted with the primer-nucleic acid template complex, such that the second nucleotide binds to the second nucleotide, the polymerase, and the primer-nucleic acid template complex to form a structurally larger complex structure. The solution containing the second nucleotide and the polymerase may further contain a buffer, a divalent metal ion, an ammonium ion, etc.

Unlike step (b11), the non-polymerization reaction conditions in step (b12) refer to conditions that are not suitable for a polymerization reaction to occur, and may include the lack of any one or more reaction conditions among the necessary conditions for a polymerization reaction, which may be one or more of a temperature condition, a pH condition, a liquid environment condition, etc. Hence, the non-polymerization reaction conditions function to prevent the polymerase from promoting the polymerization reaction between the nucleotide and the reactive group at the 3′ end of the primer strand of the primer-nucleic acid template complex. Illustratively, the non-polymerization reaction conditions are conditions without Mg²⁺ and/or Mn²⁺. In some embodiments, at least one of e.g., Ca²⁺, Ni²⁺, and Si²⁺ may be used to replace Mg²⁺ and Mn²⁺ in the polymerization reaction conditions containing Mg²⁺ and/or Mn²⁺, such that the second nucleotide cannot undergoes a polymerization reaction with the reactive group at the 3′ end of the primer strand of the primer-nucleic acid template complex.

In some examples, step (b12) further comprises: washing to remove the second nucleotide which has not been reacted.

In one embodiment, the substrate is iii), comprising a third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand. The fluorescently labeled antibody provided in examples of the present application comprises an antibody, and a fluorescent label bound to the antibody, the fluorescent label being used to identify the third nucleotide; in this case, step (b1) is (b13). In step (b13), the third nucleotide, the polymerase and the primer-nucleic acid template complex are contacted under conditions suitable for a polymerization reaction to occur. The reactive group at the 3′ end of the primer strand of the primer-nucleic acid template complex, i.e., the 3′ end of the first nucleic acid segment, is polymerized with the third nucleotide, such that the third nucleotide is incorporated into the 3′ end of the primer strand of the primer-nucleic acid template complex, thereby achieving the extension of the 3′ end of the primer strand. The fluorescently labeled antibody forms a larger complex structure with the extension product by means of binding to the blocking group and base of the third nucleotide. The type of third nucleotide incorporated at the 3′ end of the primer strand of the primer-nucleic acid template complex can be determined by performing signal acquisition and identification on the fluorescent label of the fluorescently labeled antibody in the complex structure.

In this example, the third nucleotide includes fluorescent label-free nucleotide dATP or an analog thereof, fluorescent label-free nucleotide dTTP or an analog thereof, fluorescent label-free nucleotide dGTP or an analog thereof, and fluorescent label-free nucleotide dCTP or an analog thereof. In step (b13), the four kinds of fluorescent label-free nucleotides may be added at the same time; or, the four kinds of fluorescent label-free nucleotides may be grouped into two combinations, with one combination containing two of the fluorescent label-free nucleotides and the other combination containing the other two of the fluorescent label-free nucleotides, or one combination containing one of the fluorescent label-free nucleotides and the other combination containing the other three of the fluorescent label-free nucleotides, so as to add the four kinds of fluorescent label-free nucleotides in two instances; or, of course, the four kinds of fluorescent label-free nucleotides may also be added separately in four instances. One instance of addition refers to a process in which the third nucleotide and the polymerase are added such that one third nucleotide is incorporated into the 3′ end of the primer strand of at least one primer-nucleic acid template complex, and signal acquisition is achieved by means of the fluorescently labeled antibody bound to the third nucleotide. In one example, step (b13) and step (c1) described below are regarded as a cycle of one instance of addition. In some examples, for the sake of sequencing efficiency, in step (b13), the four kinds of fluorescent label-free nucleotides are added at the same time.

In examples of the present application, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the third nucleotide, the fluorescently labeled antibody and the polymerase is contacted with the primer-nucleic acid template complex, such that the third nucleotide is incorporated into the 3′ end of the primer strand of the primer-nucleic acid template complex, and the fluorescently labeled antibody binds to the third nucleotide of the extension product. The solution containing the third nucleotide, the fluorescently labeled antibody and the polymerase may further contain a buffer, a divalent metal ion, a magnesium ion, an ammonium ion, etc., wherein the divalent metal ion may be at least one of Mg²⁺ and/or Mn²⁺, so as to promote the occurrence of the polymerization reaction.

In step (b13), the third nucleotide, the fluorescently labeled antibody, the polymerase, and the primer-nucleic acid template complex are contacted under conditions suitable for a polymerization reaction to occur, such that one third nucleotide is incorporated into the 3′ end of the primer strand of at least one primer-nucleic acid template complex under the action of the polymerase. The conditions suitable for a polymerization reaction to occur refer to conditions capable of initiating a polymerization reaction between a phosphoric acid group of the nucleotide (in step (b13), the third nucleotide) and the reactive group at the 3′ end of the primer strand. In this example, the polymerization reaction conditions can also be suitable for the binding of the fluorescently labeled antibody to the third nucleotide. In some examples, the conditions suitable for a polymerization reaction to occur include one or two of the following conditions 1) and 2):
1) A reaction temperature of 30° C. to 75° C.; and
3) A pH of 8.0 to 10.

Illustratively, the foregoing reaction temperature may specifically be 30° C., 32° C., 35° C., 36° C., 37° C., 38° C., 39° C., 40° C., 41° C., 42° C., 43° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the foregoing pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application.

The described reaction temperature and pH cover relatively mild reaction conditions, for example, a reaction temperature of 30° C. to 55° C., and a pH of 8.0 to 8.5. However, it is worth noting that the present application uses the described sequencing primer to bind to the nucleic acid template, which improves the stability of the primer-nucleic acid template complex. On that basis, the polymerization reaction can be carried out under higher temperature and pH conditions, and the intactness and stability of the double-stranded structure can still be maintained. In some examples, the conditions for the polymerization reaction include: a reaction condition of a pH of 9.1 to 10.0, or a reaction condition of a temperature of 60° C. to 75° C., or reaction conditions of a pH of 9.1 to 10.0 and a temperature of 60° C. to 75° C. In some examples, the conditions for the polymerization reaction include: a reaction condition of a pH of 9.8 to 10.0, or a reaction condition of a temperature of 65° C. to 75° C., or reaction conditions of a pH of 9.8 to 10.0 and a temperature of 65° C. to 75° C.

In some examples, the polymerase is Phi29 or Klenow or a mutant thereof, and the temperature suitable for the polymerization reaction to occur is 30° C. to 45° C. In some examples, the polymerase is 9° N or KOD or a mutant thereof, and the temperature suitable for the polymerization reaction to occur is 50° C. to 75° C.

In some examples, step (b13) further comprises: washing to remove the third nucleotide which has not been reacted, and the fluorescently labeled antibody.
(c1) Exciting the fluorescent label to generate a fluorescence signal, and collecting a fluorescence image.

In this step, excitation light excites the fluorescent label in the first nucleotide, the second nucleotide, and the fluorescently labeled antibody, to generate a fluorescence signals. The fluorescence signal generated from the fluorescent labels of the four kinds of nucleotides or the fluorescently labeled antibody is collected. It should be understood that the wavelength of the excitation light required to generate an optical signal may be different for different fluorescent labels. Hence, different fluorescent labels may be excited using excitation light of different wavelengths respectively to generate fluorescence signals, and after each instance of excitation, a fluorescence signal is collected.

Within each imaging region, the fluorescence signals of the four kinds of nucleotides or the fluorescently labeled antibody bound with the four kinds of nucleotides constitute a fluorescence image.

When step (b1) is (b11), step (c1) is (c11).

Step (c11) further comprises, determining, based on the fluorescence image, the type of the first nucleotide incorporated into the primer strand. In some examples, the type of the incorporated first nucleotide is identified on the basis of a bright spot feature in the fluorescence image. The bright spot feature comprises the color of a bright spot and the intensity of a bright spot.

Further, after signal collection is completed, the step further comprises (d11): removing the blocking group of the first nucleotide to enable another incorporation of one first nucleotide in a next round of step (b11).

When step (b1) is (b12), step (c1) is (c12). Step (c12) further comprises: determining, based on the fluorescence image, the type of the second nucleotide bound to the polymerase, and further determining the type of a next nucleotide incorporated into the 3′ end of the primer strand of the primer-nucleic acid template complex. In some examples, the type of the nucleotide or the analog thereof bound to the polymerase is identified on the basis of a bright spot feature in the fluorescence image. The bright spot feature comprises the color of a bright spot and the intensity of a bright spot.

After step (c12), further comprised is step (d12): removing the second nucleotide. In some examples, the second nucleotide is removed by means adding a chelating agent and performing rinsing. The chelating agent can chelate the divalent metal ion, to dissociate the complex structure formed by the binding of the second nucleotide, the polymerase and the primer-nucleic acid template complex. In some examples, the removal of the second nucleotide can be achieved by means of converting the non-polymerization reaction conditions to polymerization reaction conditions. Illustratively, Mg²⁺ and/or Mn²⁺ are added to replace the Ca²⁺, Ni²⁺, Si²⁺, etc. originally contained in the solution, and the third nucleotide is added, such that the third nucleotide undergoes a polymerization reaction with the reactive group at the 3′ end of the primer strand of the primer-nucleic acid template complex under the action of the polymerase, thereby removing the second nucleotide.

Step (d12) involves contacting the third nucleotide, i.e., a nucleotide having a blocking group, or an analog thereof, with the primer-nucleic acid template complex under conditions suitable for the polymerization reaction, such that the third nucleotide is incorporated into the 3′ end of the primer strand of the primer-nucleic acid template complex.

In step (d12), the third nucleotide refers to a nucleotide or an analog thereof having, in the structure thereof, a blocking group capable of blocking the formation of a phosphate ester bond between the 3′-OH of the five-carbon sugar in the nucleotide or the analog thereof and a phosphate group of another nucleotide. The nucleotide or the analog thereof having a blocking group may not comprise a fluorescent label.

In step (d12), the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution of the third nucleotide and the polymerase is contacted with the primer-nucleic acid template complex, such that the third nucleotide is incorporated into the 3′ end of the primer strand of the primer-nucleic acid template complex under the action of the polymerase.

In some examples, a solution containing the third nucleotide and the polymerase is contacted with the primer-nucleic acid template complex, such that the third nucleotide is incorporated into the 3′ end of the primer strand of the primer-nucleic acid template complex. The solution containing the third nucleotide and the polymerase may further contain a buffer, a divalent metal ion, an ammonium ion, etc., wherein the divalent metal ion may be at least one of Mg²⁺ and/or Mn²⁺, so as to promote the occurrence of the polymerization reaction.

In step (d12), the third nucleotide, the polymerase, and the primer-nucleic acid template complex are contacted under conditions suitable for a polymerization reaction to occur, such that one nucleotide (third nucleotide) having a blocking group is incorporated into the 3′ end of the primer strand of at least one primer-nucleic acid template complex under the action of the polymerase. The conditions suitable for a polymerization reaction to occur refer to conditions capable of initiating a polymerization reaction between a phosphoric acid group of the nucleotide having a blocking group and the reactive group at the 3′ end of the primer strand. In some examples, the conditions suitable for a polymerization reaction to occur include one or two of the following conditions 1) and 2):
1) A reaction temperature of 30° C. to 75° C.; and
4) A pH of 8.0 to 10.

Illustratively, the foregoing reaction temperature may specifically be 30° C., 32° C., 35° C., 36° C., 37° C., 38° C., 39° C., 40° C., 41° C., 42° C., 43° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the foregoing pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application.

The described reaction temperature and pH cover relatively mild reaction conditions, for example, a reaction temperature of 30° C. to 55° C., and a pH of 8.0 to 8.5. However, it is worth noting that the present application uses the described sequencing primer to bind to the nucleic acid template, which improves the stability of the primer-nucleic acid template complex. On that basis, the polymerization reaction can be carried out under higher temperature and pH conditions, and the intactness and stability of the double-stranded structure can still be maintained. In some examples, the conditions for the polymerization reaction include: a reaction condition of a pH of 9.1 to 10.0, or a reaction condition of a temperature of 60° C. to 75° C., or reaction conditions of a pH of 9.1 to 10.0 and a temperature of 60° C. to 75° C. In some examples, the conditions for the polymerization reaction include: a reaction condition of a pH of 9.8 to 10.0, or a reaction condition of a temperature of 65° C. to 75° C., or reaction conditions of a pH of 9.8 to 10.0 and a temperature of 65° C. to 75° C.

In some examples, the polymerase is Phi29 or Klenow or a mutant thereof, and the temperature suitable for the polymerization reaction to occur is 30° C. to 45° C. In some examples, the polymerase is 9° N or KOD or a mutant thereof, and the temperature suitable for the polymerization reaction to occur is 50° C. to 75° C.

In some examples, the step further comprises: washing to remove the third nucleotide which has not been reacted.

When step (b1) is (b13), step (c1) is (c13). Step (c13) further comprises: determining, based on the fluorescence image, the type of the third nucleotide bound to the polymerase. In some examples, the type of the third nucleotide bound to the fluorescently labeled antibody is identified on the basis of a bright spot feature in the fluorescence image. The bright spot feature comprises the color of a bright spot and the intensity of a bright spot.

In some examples, after step (c13), further comprised is step (d13): removing the fluorescently labeled antibody. In some examples, the fluorescently labeled antibody can be removed by heating, or the fluorescently labeled antibody bound to the extension product can be unbound by adding the third nucleotide and the polymerase.
(e1) Repeating step (b1) to step (c1) one or more times.

In this step, in each instance of step (b1) to step (c1), the extension of one nucleotide or one analog thereof at the 3′ end of the primer strand is achieved, and the type of the nucleotide or the analog thereof incorporated into the primer strand is confirmed. By repeating multiple times, determination of the sequence of the inserted nucleic acid fragment in the nucleic acid template sequence is achieved.

When the sequencing method comprises step (b11), step (e1) involves repeating step (b11) to step (d11) one or more times, so as to determine the sequence of the nucleic acid template. When the sequencing method comprises step (b12), step (e1) involves repeating step (b12) to step (d12) one or more times, so as to determine the sequence of the nucleic acid template. When the sequencing method comprises step (b13), step (e1) involves repeating step (b13) to step (d13) one or more times, so as to determine the sequence of the nucleic acid template.

In the described sequencing method, since a tripartite primer comprising a first nucleic acid segment, a linker and a second nucleic acid segment is introduced as a sequencing primer to improve the stability of a double-stranded structure formed by the primer and a nucleic acid template, conditions for a sequencing reaction can be advantageously expanded, including selecting harsher polymerization reaction conditions, so as to accelerate the progress of the sequencing reaction or improve the level of one or more sequencing indexes of the sequencing reaction, without damaging the stability of the double-stranded structure.

A second sequencing method provided by examples of the present application comprises:
(a2) Hybridizing a first sequencing primer and a second sequencing primer to a cluster of nucleic acid templates to form primer-nucleic acid template complexes.

In this step, referring to FIG. 5 or FIG. 6, the cluster of nucleic acid templates is a cluster of clones formed from nucleic acid templates, including multiple nucleic acid templates having the same sequence, and the multiple nucleic acid templates having the same sequence at least comprise a first nucleic acid template and a second nucleic acid template having the same sequence. These nucleic acid templates each comprise a first inserted nucleic acid fragment and a second inserted nucleic acid fragment, the sequence of which is to be determined, the first inserted nucleic acid fragment being located at the 3′ end of the second inserted nucleic acid fragment. In some examples, the first inserted nucleic acid fragment and the second inserted nucleic acid fragment are two segments of nucleic acid molecule, the sequence of which is unknown, such as two nucleic acid sequences to be determined. In some examples, one of the first inserted nucleic acid fragment and the second inserted nucleic acid fragment is a nucleic acid molecule, the sequence of which is unknown, and the other is a nucleic acid molecule, the sequence of which is to be determined. By a nucleic acid molecule whose sequence is to be determined, what is meant is that the nucleic acid molecule is selected from one of multiple nucleic acid molecules having known sequences, but it is uncertain which of the multiple known sequences the nucleic acid molecule specifically is. Therefore, the sequence needs to be sequenced so as to determine the identity thereof. In some examples, the nucleic acid molecule the sequence of which is to be determined is a tag sequence, i.e., an Index sequence.

In examples of the present application, the 3′ end of the first inserted nucleic acid fragment in the nucleic acid template is further provided with a first primer binding site, the first primer binding site having a known sequence. The first primer binding site is used for the binding of a sequencing primer that extends a complementary strand of the first inserted nucleic acid fragment, so as to achieve sequencing of the first inserted nucleic acid fragment. In some examples, the first primer binding site is a first adaptor. In some embodiments, the first adaptor serves as an amplification primer for amplification of the inserted nucleic acid fragment. Illustratively, the first adaptor is a complementary sequence of P5 or a complementary sequence of P7, and is used for hybridizing with P5 or P7 to achieve amplification of a nucleic acid molecule comprising the inserted nucleic acid fragment, such as bridge amplification. In some examples, a polynucleotide sequence is provided at the 5′ end of the second inserted nucleic acid fragment and is used for fixing or linking the inserted nucleic acid fragment. In some examples, the polynucleotide sequence at the 5′ end of the second inserted nucleic acid fragment is a second adaptor. In some embodiments, the second adaptor serves as an amplification primer for amplification of the inserted nucleic acid fragment. Illustratively, the second adaptor is P5 or P7, and is used as an amplification primer to achieve amplification of a nucleic acid molecule comprising the inserted nucleic acid fragment, such as bridge amplification. In one example, the combination of the polynucleotide sequence at the 5′ end of the second inserted nucleic acid fragment and the known sequence is a combination formed of a complementary sequence of P7 and P5, or a combination formed of a complementary sequence of P5 and P7.

In examples of the present application, a second primer binding site is provided between the two inserted fragments of the nucleic acid template, and the second primer binding site has a known sequence, but the sequence of the first primer binding site is different from that of the second primer binding site. The second primer binding site is linked to the 3′ end of the second inserted nucleic acid fragment, and is used for the binding of a sequencing primer that extends a complementary strand of the second inserted nucleic acid fragment, so as to achieve sequencing of the second inserted nucleic acid fragment.

In a specific example, from the 3′ end to the 5′ end, the multiple number of nucleic acid templates (at least including first nucleic acid templates and second nucleic acid templates) having the same sequence comprises the first primer binding site, the first inserted nucleic acid fragment, the second primer binding site, the second inserted nucleic acid fragment, and the polynucleotide.

In some examples, the cluster of nucleic acid templates provided in examples of the present application is fixed on a surface of a solid phase substrate via the 5′ end, and the method of fixing is not strictly limited. In one implementation, the cluster of clones may be formed by means such as bridge amplification. In one example, the nucleic acid templates in at least one cluster of clones at least comprise, from the 3′ end to the 5′ end, the first primer binding site, the first inserted nucleic acid fragment, the second primer binding site, the second inserted nucleic acid fragment and the polynucleotide, and the polynucleotide is linked to a surface of the solid phase substrate.

In examples of the present application, the first sequencing primer is the sequencing primer (tripartite primer) described above, comprising the linker, the first nucleic acid segment and the second nucleic acid segment. The first nucleic acid segment is located at the 3′ end of the first sequencing primer, and the 3′ end of the first nucleic acid segment has a reactive group. In examples of the present application, the reactive group at the 3′ end of the first nucleic acid segment has reactivity to undergo a polymerization reaction with a nucleotide or an analog thereof, such that the first nucleic acid segment can be extended at the 3′ end by means of the polymerization reaction. In some embodiments, the reactive group comprises —OH. In this case, the —OH at the 3′ end of the first nucleic acid segment reacts with a phosphate group of the nucleotide or the analog thereof to form a phosphate ester group, thereby achieving the extension of the nucleotide or the analog thereof. As part of the first sequencing primer, the first nucleic acid segment is configured to be partially complementary or completely complementary to a sequence of the sequencing primer binding site of the nucleic acid template, and can undergo a polymerization reaction with the nucleotide or the analog thereof via the reactive group at the 3′ end to achieve extension of the primer strand from the 5′ end to the 3′ end. In one example, the first nucleic acid segment is configured to be completely complementary to a sequence of one sequencing primer binding site of the nucleic acid template.

The second nucleic acid segment is located at the 5′ end of the first sequencing primer, and is configured to be partially complementary or completely complementary to a sequence of one sequencing primer binding site of the target nucleic acid molecule. In one embodiment, the 3′ end of the second nucleic acid segment is linked to the linker, therefore the second nucleic acid segment bound to the sequencing primer binding site does not have an extension property, such that the sequencing primer binding site bound thereto is blocked. In another embodiment, the 5′ end of the second nucleic acid segment is linked to the linker, and at the same time, the second nucleic acid segment has a 3′ blocked end, and therefore the second nucleic acid segment bound to the sequencing primer binding site does not have a 5′ to 3′ extension property either, such that the sequencing primer binding site bound thereto is blocked. The 3′ occluded end can be achieved in various ways. For example, a blocking group that does not undergo a polymerization reaction with a nucleotides or an analog thereof, and particularly does not undergo a polymerization reaction with a phosphoric acid of the nucleotide or the analog thereof, is formed at the 3′ end, including but not limited to 3′ amino blocking, or using a dideoxynucleotide or an analogs thereof as the terminal nucleotide of the second nucleic acid segment, etc. In one example, the second nucleic acid segment is configured to be completely complementary to a sequence of one sequencing primer binding site of the nucleic acid template.

In this example, the first nucleic acid segment and the second nucleic acid segment are used to bind to the same sequencing primer binding sites in the nucleic acid templates of the cluster of clones, so as to block a portion of the same sequencing primer binding sites in the cluster of clones. Therefore, the sequence of the first nucleic acid segment is identical or complementary to the sequence of the second nucleic acid segment. In one embodiment, the first nucleic acid segment is partially complementary or completely complementary to the first primer binding sites of the first nucleic acid templates, and the second nucleic acid segment is partially complementary or completely complementary to the first primer binding sites of the second nucleic acid templates, such that a portion of the first primer binding sites are blocked. Hence, the number of extendable primers bound to the first primer binding sites is less than the number of extendable primers bound to the second primer binding sites, such that the sequence signals generated by the extension of the two kinds of primer strand in the same cluster of clones become different. In one embodiment, the first nucleic acid segment is partially complementary or completely complementary to the second primer binding sites of the first nucleic acid templates, and the second nucleic acid segment is partially complementary or completely complementary to the second primer binding sites of the second nucleic acid templates, such that a portion of the second primer binding sites are blocked. Hence, the number of extendable primers bound to the second primer binding sites is less than the number of extendable primers bound to the second primer binding sites, such that the sequence signals generated by the extension of the two kinds of primer strand in the same cluster of clones become different. In the case that both the first nucleic acid segment and the second nucleic acid segment are partially complementary or completely complementary to the second primer binding sites, the linker between the first nucleic acid segment and the second nucleic acid segment may constitute a certain spatial barrier to the sequencing of the first inserted nucleic acid fragment. In contrast, in the case that both the first nucleic acid segment and the second nucleic acid segment are partially complementary or completely complementary to the first primer binding sites, the linker is located at the 3′ end of the cluster clones, and sequencing will not be influenced because of the linker occupying a region of the two inserted nucleic acid fragments.

In examples of the present application, in the first sequencing primer, the number of the first nucleic acid segments and the number of the second nucleic acid segments and the ratio thereof can be adjusted within a certain range. In some examples, in each first sequencing primer, the molar ratio of the first nucleic acid segment to the second nucleic acid segment is 1:5 to 5:1. In this case, the ratio of the number of blocked first primer binding sites to the total number of first primer binding sites (which is also the total number of second primer binding sites) in the cluster of clones can be increased, and the sequencing signals generated by the number of extendable primers bound to the two primer binding sites during extension become more obviously different, which is beneficial to the identification of the two kinds of sequencing signal. Of course, in this way, the number of first nucleic acid segments linked by means of the linker and the spatial hindrance caused by the second nucleic acid segment can also be adjusted. Illustratively, in each first sequencing primer, the molar ratio of the first nucleic acid segment to the second nucleic acid segment may specifically be 1:5, 1:4, 1:3, 1:2, 1:1, 2:1, 3:1, 4:1, 5:1, etc. Illustratively, in each first sequencing primer, the number of first nucleic acid segments and the number of second nucleic acid segments are both 1. In examples of the present application, the difference in sequencing signal includes the difference in fluorescence color and the difference in fluorescence intensity. By virtue of the difference in fluorescence color and the difference in fluorescence intensity, synchronous sequencing of the two inserted nucleic acid fragments can be achieved.

In this example, the linker is used to link the first nucleic acid segment and the second nucleic acid segment together, for the spatial distance between the first nucleic acid template and the second nucleic acid template in the cluster of clones. Specifically, the linker is bound to the 5′ end of the first nucleic acid segment and the 3′ end or 5′ end of the second nucleic acid segment, respectively, and in the case that the linker is bound to the 3′ end of the second nucleic acid segment, the second nucleic acid segment has a 3′ occluded end.

In some embodiments, the linker has a length of 0.3 nm to 100 nm to enable spanning over a certain spatial distance. Illustratively, the length of the linker may be in the range of 0.3 nm to 1 nm, 0.3 nm to 5 nm, 0.3 nm to 10 nm, 1 nm to 10 nm, 1 nm to 20 nm, 5 nm to 20 nm, 10 nm to 20 nm, 10 nm to 50 nm, 20 nm to 50 nm, 30 nm to 50 nm, 50 nm to 100 nm, 50 nm to 60 nm, 50 nm to 80 nm, etc. In examples of the present application, the type of the linker may be selected from a variety of options and is not strictly limited. In one embodiment, the linker comprises one or multiple hydrophilic groups. The hydrophilic groups include, but are not limited to, a hydroxyl group, a carboxyl group, a sulfonate group, an ester group, a phosphate ester group, an amino group, an ether group, and an amide group.

In some embodiments, the linker comprises at least one selected from an optionally substituted or unsubstituted C5-C20 alkylene, a C5-C20 alkylene in which one or more carbon atoms are substituted by a heteroatom, a small molecule compound, a polymer, a peptide chain, a polynucleotide, a polysaccharide, and an aromatic ring.

In some specific embodiments of the present invention, the linker is selected from a polymer, the number of backbone atoms in the polymer being greater or equal to than 300 atoms, and a molecular weight thereof being greater than or equal to 4,000.

In some examples, the polymer comprises at least one of a hydroxyl group, a carboxyl group, a sulfonate group, an ester group, a phosphate group, an amino group, an ether group, and an amide group. In one aspect, these groups have good hydrophilicity; in another aspect, these groups can impart good flexibility to the linker, to achieve spanning thereof over a nucleic acid sequence and/or space.

In examples of the present application, the polymer that can be used as the linker comprises, but is not limited to, at least one of polyethylene glycol, polyvinyl alcohol, polyacrylamide, polymethacrylic acid, polyvinyl sulfonic acid, and polyvinylpyrrolidone. Illustratively, the polymer is at least one of iSp18 (polyethylene glycol 6), polyethylene glycol 200, polyethylene glycol 800, polyethylene glycol 2000, and polyethylene glycol 4000.

In some embodiments, the linker is selected from a small molecule compound the number of backbone atoms in which is greater than or equal to 6 and a molecular weight of which is no greater than 1,000.

In some specific embodiments of the present invention, the linker is a polynucleotide, and the length of the polynucleotide is greater than the length of the spacer sequence. Illustratively, when the spacer sequence is an Index sequence, the length of the linker is greater than the length of the Index sequence. In some examples, the GC content in the polynucleotide is 0% to 70%, which can reduce the influence of excessive hydrogen bonds on the flexibility of the linker. Of course, an excessively high GC content, such as a GC content of more than 70%, provides a favorable condition for the formation of a G-quadruplex, and a large number of G-quadruplex structures will appear in the polynucleotide structure, converting the linker into a more rigid structure.

In some embodiments, the linker is selected from a nucleotide or polynucleotide composed of 1 to 40 bases, preferably a polynucleotide composed of 3 to 26 bases.

In some embodiments, the linker is bound to the 5′ end of the first nucleic acid segment through a first linking group, and is bound to the 3′ end of the second nucleic acid segment through a second linking group. In examples of the present application, one or a multiple number of first linking groups are present in the linker. When multiple first linking groups are present in the linker, the multiple first linking groups may be bound to the 5′ end of one first nucleic acid segment, or may be bound to the 5′ ends of multiple first nucleic acid segments. The manner in which the first linking group may be bound to the 5′ end of the first nucleic acid segment includes chemical bonding, hydrogen bond formation, etc. Illustratively, the first linking group is selected from a hydroxyl group, a carboxyl group, a sulfonate group, an ester group, a phosphate ester group, an amino group, an ether group, a saccharide group, an alkyl group, an amide group, and aromatic ring, or a biological macromolecule having a molecular weight of not more than 100,000. Illustratively, the biological macromolecule is selected from a polysaccharide, a polypeptide chain, and an oligomeric nucleic acid.

In examples of the present application, one or multiple second linking groups are present in the linker. When multiple second linking groups are present in the linker, the multiple second linking groups may be bound to the 3′ end of one second nucleic acid segment, or may be bound to the 3′ ends of a multiple number of the second nucleic acid segments. The manner in which the second linking group may be bound to the 5′ end of the second nucleic acid segment includes chemical bonding, hydrogen bond formation, etc. Illustratively, the second linking group is selected from a hydroxyl group, a carboxyl group, a sulfonate group, an ester group, a phosphate ester group, an amino group, an ether group, a saccharide group, an alkyl group, an amide group, and aromatic ring, or a biological macromolecule having a molecular weight of not more than 100,000. Illustratively, the biological macromolecule is selected from a polysaccharide, a polypeptide chain, and an oligomeric nucleic acid.

In examples of the present application, one first sequencing primer may only comprise one first nucleic acid segment, and the 5′ end of the first nucleic acid segment is linked to the linker. One first sequencing primer may also comprise two or more first nucleic acid segments, and the 5′ ends of the multiple first nucleic acid segments are linked to the linker via a multiple number of first linking groups on the same linker.

In examples of the present application, one first sequencing primer may only comprise one second nucleic acid segment, and the 3′ end of the second nucleic acid segment is linked to the linker. One first sequencing primer may also comprise two or more second nucleic acid segments, and the 3′ ends of the multiple second nucleic acid segments are linked to the linker by means of multiple first linking groups on the same linker, or the multiple second nucleic acid segments may also be sequentially linked by means of multiple linkers.

Of course, one first sequencing primer may comprise one or multiple first nucleic acid segments and at the same time comprise one or multiple second nucleic acid segments. Illustratively, one first sequencing primer comprises one first nucleic acid segment and and one second nucleic acid segment; or, one first sequencing primer comprises two or more first nucleic acid segments and one second nucleic acid segment; or, one first sequencing primer comprises one first nucleic acid segment and two or more second nucleic acid segments; or, one first sequencing primer comprises two or more first nucleic acid segments and two or more second nucleic acid segments at the same time.

In one example, each first sequencing primer comprises X first nucleic acid segments, and the X first nucleic acid segments are partially complementary or completely complementary to the sequencing primer binding sites of X nucleic acid templates, respectively, wherein X is a positive integer greater than 1 and less than 10. Illustratively, X is selected from 1, 2, 3, 4, 5, 6, 7, 8, etc. In one example, the number of first nucleic acid segments is equal to the number of second nucleic acid segments. Illustratively, in each sequencing primer, the number of first nucleic acid segments and the number of second nucleic acid segments are both 1.

In some examples, each first sequencing primer comprises N first nucleic acid segments, and the N first nucleic acid segments are linked by means of one linker, wherein N is a positive integer less than or equal to 10. For example, N is selected from 1, 2, 3, 4, 5, 6, 7, 8, etc. In one example, each first sequencing primer comprises N first nucleic acid segments, the 5′ ends of the first nucleic acid segments each have a first linking group, and the N first nucleic acid segments are linked to one linker by means of N first linking groups, respectively. In some examples, each first sequencing primer comprises M second nucleic acid segments, and the M second nucleic acid segments are sequentially linked by means of 1 to M linkers, wherein M is a positive integer less than or equal to 10. For example, M is selected from 1, 2, 3, 4, 5, 6, 7, 8, etc. In one example, each first sequencing primer comprises M second nucleic acid segments, and the M second nucleic acid segments are linked by means of M linkers, respectively, wherein M is a positive integer less than or equal to 10. In one example, M=N=1.

In examples of the present application, the second sequencing primer is configured to be partially complementary or completely complementary to a sequence of the second primer binding site in the nucleic acid templates (including first nucleic acid templates and second nucleic acid templates) in the cluster of clones. In one example, the second sequencing primer is completely complementary to a sequence of the second primer binding site in the nucleic acid templates (including first nucleic acid templates and second nucleic acid templates) in the cluster of clones. Moreover, the 3′ end of the second sequencing primer has a reactive group, which allows chain extension with a nucleotide or an analog thereof through a polymerization reaction after binding to the second primer binding site.

In this step, the first sequencing primer and the second sequencing primer are contacted with the cluster of nucleic acid templates. The first nucleic acid segment in the first sequencing primer is partially complementary or completely complementary to the first sequencing primer binding sites of the first nucleic acid templates, and the second nucleic acid segment is partially complementary or completely complementary to the first sequencing primer binding sites of the second nucleic acid templates, so as to achieve hybridization of the sequencing primers and the nucleic acid templates to obtain primer-nucleic acid template complexes. In the primer-nucleic acid template complexes thus obtained, two primer strands are bound onto the nucleic acid template molecules. A portion of the primer-nucleic acid template complexes comprise the first nucleic acid segment and the second sequencing primer which are bound onto the nucleic acid template molecules, and are referred to herein as a first kind of primer-nucleic acid template complex; another portion of the primer-nucleic acid template complexes comprise the second nucleic acid segment and the second sequencing primer which are bound onto the nucleic acid template molecules, and are referred to herein as a second kind of primer-nucleic acid template complex. The first nucleic acid segment and the second sequencing primer of the first kind of primer-nucleic acid template complex are capable of being extended by means of polymerizing a nucleotide or an analog thereof, the second nucleic acid segment of the second kind of primer-nucleic acid template complex is incapable of being extended due to the inability to polymerize a nucleotide or an analog thereof, and the second sequencing primer of the second kind of primer-nucleic acid template complex is capable of being extended by means of polymerizing a nucleotide or an analog thereof.
(b2) Contacting one of the following substrates or substrate combinations i) to iii) and a polymerase with the primer-nucleic acid template complexes, such that the substrate or substrate combination is bound to the polymerase and the primer-nucleic acid template complexes and/or the substrate or substrate combination is incorporated into the 3′ end of the primer strand of the primer-nucleic acid template complexes,
iv) A first nucleotide, the first nucleotide comprising a fluorescent label and a blocking group used to prevent the incorporation of a next nucleotide or an analog thereof into the primer strand,
v) A second nucleotide, the second nucleotide being a nucleotide containing a fluorescent label or an analog thereof, and

A third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand.

In this step, the first nucleotide, the second nucleotide and the fluorescently labeled antibody comprises a fluorescent label, the first nucleotide polymerizes with the primer-nucleic acid template complexes under the action of the polymerase, and the second nucleotide and the fluorescently labeled antibody can bind to the primer-nucleic acid template complexes. The type of the nucleotide incorporated upon primer strand extension can be determined by means of determining the fluorescent label in the fluorescently labeled antibody or nucleotide that is bound to or polymerized with the primer-nucleic acid template complexes.

In one embodiment, the substrate is i), i.e., a first nucleotide, the first nucleotide comprising a fluorescent label and a blocking group used to prevent the incorporation of a next nucleotide or an analog thereof into the primer strand, and in this case, step (b2) is (b21). In step (b21), the first nucleotide, the polymerase and the primer-nucleic acid template complexes are contacted under conditions suitable for a polymerization reaction to occur. The reactive group at the 3′ end of the primer strand of the primer-nucleic acid template complexes, i.e., the 3′ end of the first nucleic acid segment, and the reactive group at the 3′ end of the second sequencing primer each polymerize with the first nucleotide, such that the first nucleotide is incorporated into the 3′ ends of the primer strands of the primer-nucleic acid template complexes, thereby achieving the extension of the 3′ ends of the two primer strands (the first sequencing primer strand and the second sequencing primer strand). The type of first nucleotide incorporated at the 3′ ends of the two primer strands of the primer-nucleic acid template complexes can be separately determined by performing signal acquisition and identification on the fluorescent label of the first nucleotide. In addition, since the first nucleotide further comprises a blocking group used to prevent the incorporation of another nucleotide into the primer strands, in each step (b21), the 3′ ends of the primer strands tend to introduce a nucleotide reversible terminator, so as to extend one nucleotide.

In this example, the nucleotide reversible terminator includes reversible terminator dATP, reversible terminator dTTP, reversible terminator dGTP and reversible terminator dCTP. In step (b21), the reversible terminator dATP, reversible terminator dTTP (or reversible terminator dUTP), reversible terminator dGTP, and reversible terminator dCTP may be added at the same time; or, the four kinds of reversible terminator may be grouped into two combinations, with one combination containing two of the reversible terminators and the other combination containing the other two of the reversible terminators, or one combination containing one of the reversible terminators and the other combination containing the other three of the reversible terminators, so as to add the four kinds of reversible terminator in two instances; or, of course, the four kinds of reversible terminator may also be added separately in four instances. One instance of addition refers to a process in which a reversible terminator or reversible terminators and the polymerase are added such that one nucleotide reversible terminator is incorporated into the 3′ end of the primer strand of at least one primer-nucleic acid template complex, and signal acquisition is achieved. In one example, step (b21) and step (c2) described below are regarded as a cycle of one instance of addition. In some examples, for the sake of sequencing efficiency, in step (b21), the reversible terminator dATP, reversible terminator dTTP (or reversible terminator dUTP), reversible terminator dGTP and reversible terminator dCTP are added at the same time to perform the polymerization reaction.

In this example, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the first nucleotide and the polymerase is contacted with the primer-nucleic acid template complexes, such that the first nucleotide is incorporated into the 3′ ends of the primer strands (the first sequencing primer strand and the second sequencing primer strand) of the primer-nucleic acid template complexes. The solution containing the first nucleotide and the polymerase may further contain a buffer, a divalent metal ion, an ammonium ion, etc., wherein the divalent metal ion may be at least one of Mg²⁺ and/or Mn²⁺, so as to promote the occurrence of the polymerization reaction.

In step (b21), the first nucleotide, the polymerase, and the primer-nucleic acid template complexes are contacted under conditions suitable for a polymerization reaction to occur, such that one first nucleotide is incorporated into the 3′ ends of the primer strands (including the first sequencing primer strand and the second sequencing primer strand) of at least one primer-nucleic acid template complex under the action of the polymerase. The conditions suitable for a polymerization reaction to occur refer to conditions capable of initiating a polymerization reaction between a phosphoric acid group of the first nucleotide and the reactive group at the 3′ ends of the primer strands. In some examples, the conditions suitable for a polymerization reaction to occur include one or two of the following conditions 1) and 2):
1) A reaction temperature of 30° C. to 75° C.; and
5) A pH of 8.0 to 10.

Illustratively, the foregoing reaction temperature may specifically be 30° C., 32° C., 35° C., 36° C., 37° C., 38° C., 39° C., 40° C., 41° C., 42° C., 43° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the foregoing pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application.

The described reaction temperature and pH cover relatively mild reaction conditions, for example, a reaction temperature of 30° C. to 55° C., and a pH of 8.0 to 8.5. However, it is worth noting that the present application uses the described sequencing primers to bind to the nucleic acid templates, which improves the stability of the primer-nucleic acid template complexes. On that basis, the polymerization reaction can be carried out under higher temperature and pH conditions, and the intactness and stability of the double-stranded structure can still be maintained. In some examples, the conditions for the polymerization reaction include: a reaction condition of a pH of 9.1 to 10.0, or a reaction condition of a temperature of 60° C. to 75° C., or reaction conditions of a pH of 9.1 to 10.0 and a temperature of 60° C. to 75° C. In some examples, the conditions for the polymerization reaction include: a reaction condition of a pH of 9.8 to 10.0, or a reaction condition of a temperature of 65° C. to 75° C., or reaction conditions of a pH of 9.8 to 10.0 and a temperature of 65° C. to 75° C.

In some examples, the polymerase is Phi29 or Klenow or a mutant thereof, and the temperature suitable for the polymerization reaction to occur is 30° C. to 45° C. In some examples, the polymerase is 9° N or KOD or a mutant thereof, and the temperature suitable for the polymerization reaction to occur is 50° C. to 75° C.

In some examples, step (b21) further comprises: washing to remove the first nucleotide which has not been reacted.

In one embodiment, the substrate is ii), i.e., a second nucleotide fluorescently labeled antibody or the nucleotide is a fluorescently labeled nucleotide or an analog thereof, the second nucleotide being a nucleotide containing a fluorescent label or an analog thereof, and in this case, step (b2) is (b22). In step (b22), the second nucleotide, the polymerase and the primer-nucleic acid template complexes are contacted under non-polymerization reaction conditions. Under the action of the polymerase, the second nucleotide binds to the polymerase and the primer-nucleic acid template complexes to form a structurally larger complex structure. In the complex structure, the second nucleotide does not polymerize with the primer-nucleic acid template complexes. That is, under non-polymerization conditions, no polymerization occurs between the reactive group at the 3′ end of the primer strand of the primer-nucleic acid template complexes, i.e., the 3′ end of the first nucleic acid segment, and the second nucleotide, and the second nucleotide is not incorporated into the 3′ ends of the primer strands (including the first sequencing primer strand and the second sequencing primer strand) of the primer-nucleic acid template complexes. The type of a next nucleotide incorporated at the 3′ ends of the primer strands (including the first sequencing primer strand and the second sequencing primer strand) of the primer-nucleic acid template complexes can be determined by performing signal acquisition, identification and analysis on the fluorescent label in the second nucleotide.

In some examples, since the second nucleotide does not undergo a polymerization reaction with the primer-nucleic acid template complexes, the second nucleotide may not have a blocking group used to prevent the incorporation of another nucleotide into the primer strands. In some other examples, the second nucleotide may also have a blocking group used to prevent the incorporation of another nucleotide into the primer strands.

In this example, the second nucleotide includes fluorescently labeled nucleotide dATP or an analog thereof, fluorescently labeled nucleotide dTTP or an analog thereof, fluorescently labeled nucleotide dGTP or an analog thereof, and fluorescently labeled nucleotide dCTP or an analog thereof. In step (b22), the four kinds of fluorescently labeled nucleotides may be added at the same time; or, the four kinds of fluorescently labeled nucleotides may be grouped into two combinations, with one combination containing two of the fluorescently labeled nucleotides and the other combination containing the other two of the fluorescently labeled nucleotides, or one combination containing one of the fluorescently labeled nucleotides and the other combination containing the other three of the fluorescently labeled nucleotides, so as to add the four kinds of fluorescently labeled nucleotides in two instances; or, of course, the four kinds of fluorescently labeled nucleotides may also be added separately in four instances. One instance of addition refers to a process in which a fluorescently labeled nucleotide or fluorescently labeled nucleotides and the polymerase are added such that one nucleotide reversible terminator is incorporated into the 3′ end of the primer strand of at least one primer-nucleic acid template complex, and signal acquisition is achieved. In one example, step (b22) and step (c2) described below are regarded as a cycle of one instance of addition. In some examples, for the sake of sequencing efficiency, in step (b22), the four kinds of fluorescently labeled nucleotides are added at the same time.

In this example, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the second nucleotide and the polymerase is contacted with the primer-nucleic acid template complexes, such that the second nucleotide binds to the second nucleotide, the polymerase, and the primer-nucleic acid template complexes to form a structurally larger complex structure. The solution containing the second nucleotide and the polymerase may further contain a buffer, a divalent metal ion, an ammonium ion, etc.

Unlike step (b21), the non-polymerization reaction conditions in step (b22) refer to conditions that are not suitable for a polymerization reaction to occur, and may include the lack of any one or more reaction conditions among the necessary conditions for a polymerization reaction, which may be one or more of temperature condition, pH condition, liquid environment condition, etc. Hence, the non-polymerization reaction conditions function to prevent the polymerase from promoting the polymerization reaction between the nucleotide and the reactive groups at the 3′ ends of the primer strands of the primer-nucleic acid template complexes. Illustratively, the non-polymerization reaction conditions are conditions without Mg²⁺ and/or Mn²⁺. In some embodiments, at least one of e.g., Ca²⁺, Ni²⁺, and Si²⁺ may be used to replace Mg²⁺ and Mn²⁺ in the polymerization reaction conditions containing Mg²⁺ and/or Mn²⁺, such that the second nucleotide cannot undergoes a polymerization reaction with the reactive groups at the 3′ ends of the primer strands of the primer-nucleic acid template complexes.

In some examples, step (b12) further comprises: washing to remove the second nucleotide which has not been reacted.

In one embodiment, the substrate is iii), comprising a third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strands. The fluorescently labeled antibody provided in examples of the present application comprises an antibody and a fluorescent label bound to the antibody, and the fluorescent label is used to identify the third nucleotide; in this case, step (b2) is (b23). In step (b23), the third nucleotide, the polymerase and the primer-nucleic acid template complexes are contacted under conditions suitable for a polymerization reaction to occur. The reactive groups at the 3′ ends of the primer strands of the primer-nucleic acid template complexes, i.e., the 3′ end of the first nucleic acid segment and the 3′ end of the second sequencing primer polymerize with the third nucleotide, such that the third nucleotide is incorporated into the 3′ ends of the primer strands of the primer-nucleic acid template complexes, thereby achieving the extension of the 3′ ends of the primer strands (including the first sequencing primer strand and the second sequencing primer strand). The fluorescently labeled antibody forms a larger complex structure with the extension product by binding to the blocking group and base of the third nucleotide. The type of the third nucleotide incorporated at the 3′ ends of the primer strands (including the first sequencing primer strand and the second sequencing primer strand) of the primer-nucleic acid template complexes can be respectively determined by performing signal acquisition, identification and analysis of the fluorescent label of the fluorescently labeled antibody in the complex structure.

In this example, the third nucleotide includes fluorescent label-free nucleotide dATP or an analog thereof, fluorescent label-free nucleotide dTTP or an analog thereof, fluorescent label-free nucleotide dGTP or an analog thereof, and fluorescent label-free nucleotide dCTP or an analog thereof. In step (b23), the four kinds of fluorescent label-free nucleotides may be added at the same time; or, the four kinds of fluorescent label-free nucleotides may be grouped into two combinations, with one combination containing two of the fluorescent label-free nucleotides and the other combination containing the other two of the fluorescent label-free nucleotides, or one combination containing one of the fluorescent label-free nucleotides and the other combination containing the other three of the fluorescent label-free nucleotides, so as to add the four kinds of fluorescent label-free nucleotides in two instances; or, of course, the four kinds of fluorescent label-free nucleotides may also be added separately in four instances. One instance of addition refers to a process in which the third nucleotide and the polymerase are added such that one third nucleotide is incorporated into the 3′ ends of the primer strands (including the first sequencing primer strand and the second sequencing primer strand) of at least one primer-nucleic acid template complex, and signal acquisition is achieved through the fluorescently labeled antibody bound to the third nucleotide. In one example, step (b23) and step (c2) described below are regarded as a cycle of one instance of addition. In some examples, for the sake of sequencing efficiency, in step (b23), the four kinds of fluorescent label-free nucleotides are added at the same time.

In examples of the present application, the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution containing the third nucleotide, the fluorescently labeled antibody and the polymerase is contacted with the primer-nucleic acid template complexes, such that the third nucleotide is incorporated into the 3′ ends of the primer strands (including the first sequencing primer strand and the second sequencing primer strand) of the primer-nucleic acid template complexes, and the fluorescently labeled antibody binds to the third nucleotide of the extension product. The solution containing the third nucleotide, the fluorescently labeled antibody and the polymerase may further contain a buffer, a divalent metal ion, a magnesium ion, an ammonium ion, etc., wherein the divalent metal ion may be at least one of Mg²⁺ and/or Mn²⁺, so as to promote the occurrence of the polymerization reaction. In step (b23), the third nucleotide, the fluorescently labeled antibody and the primer-nucleic acid template complexes are contacted under conditions suitable for a polymerization reaction to occur, such that one third nucleotide is incorporated into the 3′ ends of the primer strands (including the first sequencing primer strand and the second sequencing primer strand) of at least one primer-nucleic acid template complex under the action of the polymerase. The conditions suitable for a polymerization reaction to occur refer to conditions capable of initiating a polymerization reaction between a phosphoric acid group of the nucleotide (in step (b23), the third nucleotide) and the reactive groups at the 3′ ends of the primer strands. In this example, the polymerization reaction conditions can also be suitable for the binding of the fluorescently labeled antibody to the third nucleotide. In some examples, the conditions for the polymerization reaction include: a pH of 8.0 to 10.0 and/or a temperature of 30° C. to 75° C. Illustratively, the foregoing reaction temperature may specifically be 30° C., 32° C., 35° C., 36° C., 37° C., 38° C., 39° C., 40° C., 41° C., 42° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the foregoing pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application. In some examples, the conditions for the polymerization reaction include: a pH of 9.0 to 10.0 and/or a temperature of 50° C. to 75° C.

In some examples, the conditions for the polymerization reaction include: a reaction condition of a pH of 9.0 to 10.0, or a reaction condition of a temperature of 50° C. to 75° C., or reaction conditions of a pH of 9.0 to 10.0 and a temperature of 50° C. to 75° C. In some examples, the conditions for the polymerization reaction include: a reaction condition of a pH of 9.5 to 10.0, or a reaction condition of a temperature of 55° C. to 75° C., or reaction conditions of a pH of 9.5 to 10.0 and a temperature of 55° C. to 75° C.

In some examples, the polymerase is a Phi29 or Klenow mutant, and the temperature suitable for the polymerization reaction to occur is 30° C. to 45° C. In some examples, the polymerase is a 9° N or KOD mutant, and the temperature suitable for the polymerization reaction to occur is 50° C. to 75° C.

In some examples, step (b23) further comprises: washing to remove the third nucleotide which has not been reacted, and the fluorescently labeled antibody.
(c2) Exciting the fluorescent label to generate a fluorescence signal, and collecting a fluorescence image.

In this step, excitation light excites the fluorescent label in the first nucleotide, the second nucleotide, and the fluorescently labeled antibody, to generate a fluorescence signal. The fluorescence signal generated from the fluorescent labels of the four kinds of nucleotides or the fluorescently labeled antibody is collected. It should be understood that the wavelength of the excitation light required to generate an optical signal may be different for different fluorescent labels. Hence, different fluorescent labels may be excited using excitation light of different wavelengths respectively to generate a fluorescence signal, and after each excitation, the fluorescence signal is collected.

Within each imaging region, the fluorescence signals of the four kinds of nucleotides or the fluorescently labeled antibody bound with the four kinds of nucleotides constitute a fluorescence image.

In this step, further comprised is identifying the type of the incorporated nucleotide reversible terminator on the basis of a bright spot feature in the fluorescence image.

When step (b1) is (b21), step (c2) is (c21).

In some examples, after step (c21) or before step (f2), further comprised is: (d21) determining the base type of the first nucleotide of the first sequencing primer strand and of the second sequencing primer strand, respectively.

In one embodiment, (d21) comprises:
1) On the basis of the sequencing signals at one or multiple specified positions in the fluorescence image, determining a first sequencing signal component derived from the first sequencing primer strand and a second sequencing signal component derived from the second sequencing primer strand in the sequencing signals, respectively.

In this step, the specified position may be a region in the fluorescence image to which a position occupied by a cluster of nucleic acid templates on the solid phase substrate corresponds. The one or more specified positions at least comprise a first position, where fluorescent labels of the first nucleotides incorporated into the 3′ ends of the two sequencing primer strands are different, and the difference can be identified from the fluorescent image. That is, the sequencing signal at the first position comprises two fluorescence signals of different colors. The two fluorescence signals are derived from the first sequencing primer extension strand and the second sequencing primer extension strand, respectively. Since as described above, a portion of the first sequencing primer binding sites are blocked, in the two fluorescent signals, the signal with a larger sequencing signal component is classified as being derived from the second sequencing primer strand, and the signal with a smaller sequencing signal component is classified as being derived from the first sequencing primer strand. That is, the second sequencing signal component is greater than the first sequencing signal component.
2) Assigning the fluorescence signal corresponding to the first sequencing signal component in the fluorescence image obtained in step (c2) to the fluorescence signal of the first inserted nucleic acid fragment, and assigning the fluorescence signal corresponding to the second sequencing signal component to the fluorescence signal of the second inserted nucleic acid fragment.

For the fluorescence image obtained in step (c2) in each instance, the fluorescence signal corresponding to the first sequencing signal component is assigned to the fluorescence signal of the first inserted nucleic acid fragment, and the fluorescence signal corresponding to the second sequencing signal component is assigned to the fluorescence signal of the second inserted nucleic acid fragment. The fluorescence signal is fluorescence color and fluorescence intensity. Thus, the type of the nucleotide reversible terminators incorporated into the first sequencing primer strand and the second sequencing primer strand in each round of polymerization reaction can be determined.
3) On the basis of the fluorescence signals to which the first sequencing signal component and the second sequencing signal component correspond, respectively, determining the base type of each nucleotide reversible terminator incorporated into the first sequencing primer strand and the second sequencing primer strand, respectively first inserted nucleic acid fragment.

Further, after signal acquisition has been completed, the step further comprises removing the blocking group of the first nucleotide to enable another incorporation of one first nucleotide in a next round of (b1) step.

When step (b2) is (b22), step (c2) is (c22).

In some examples, after step (c22) or before step (f2), further comprised after step (d22) is: (d22) determining the base type of the second nucleotide bound to the 3′ end of the first sequencing primer strand and the 3′ end of the second sequencing primer strand, respectively. In one embodiment, (d22) comprises:
1) On the basis of the sequencing signals at one or multiple specified positions in the fluorescence image, determining a first sequencing signal component derived from the first sequencing primer strand and a second sequencing signal component derived from the second sequencing primer strand in the sequencing signals, respectively.

In this step, the specified position may be a region in the fluorescence image to which a position occupied by a cluster of nucleic acid templates on the solid phase substrate corresponds. The one or more specified positions at least comprise a first position, where fluorescent labels of the second nucleotides incorporated into the 3′ ends of the two sequencing primer strands are different, and the difference can be identified from the fluorescence image. That is, the sequencing signal at the first position includes two fluorescence signals of different colors. The two fluorescence signals are derived from the fluorescent label in the second nucleotide bound to the first sequencing primer strand and the fluorescent label in the second nucleotide bound to the second sequencing primer strand, respectively. Since, as described above, a portion of the first sequencing primer binding sites are blocked, in the two fluorescence signals, the signal with a larger sequencing signal component is classified as being derived from the second sequencing primer strand, and the signal with a smaller sequencing signal component is classified as being derived from the first sequencing primer strand. That is, the second sequencing signal component is greater than the first sequencing signal component.
2) Assigning the fluorescence signal corresponding to the first sequencing signal component in the fluorescence image obtained in step (c22) to the fluorescence signal of the first inserted nucleic acid fragment, and assigning the fluorescence signal corresponding to the second sequencing signal component to the fluorescence signal of the second inserted nucleic acid fragment.

For the fluorescence image obtained in step (c22) in each instance, the fluorescence signal corresponding to the first sequencing signal component is assigned to the fluorescence signal of the first inserted nucleic acid fragment, and the fluorescence signal corresponding to the second sequencing signal component is assigned to the fluorescence signal of the second inserted nucleic acid fragment. The fluorescence signal is fluorescence color and fluorescence intensity. Thus, the type of a next nucleotide reversible terminator (the third nucleotide) incorporated into the first sequencing primer strand and the second sequencing primer strand by means of a polymerization reaction can be determined.
3) On the basis of the fluorescence signals to which the first sequencing signal component and the second sequencing signal component correspond, respectively, determining the base type of each nucleotide reversible terminator incorporated into the first sequencing primer strand and the second sequencing primer strand, respectively first inserted nucleic acid fragment.

After step (d22), further comprised is step (e22): removing the second nucleotide. In some examples, the second nucleotide is removed by means of adding a chelating agent and performing rinsing. The chelating agent can chelate the divalent metal ion to dissociate the complex structure formed by the binding of the second nucleotide, the polymerase and the primer-nucleic acid template complexes. In some examples, the removal of the second nucleotide can be achieved by means of converting the non-polymerization reaction conditions to polymerization reaction conditions. Illustratively, Mg²⁺ and/or Mn²⁺ are added to replace the Ca²⁺, Ni²⁺, Si²⁺, etc. originally contained in the solution, and the third nucleotide is added, such that the third nucleotide undergoes a polymerization reaction with the reactive groups at the 3′ ends of the primer strands of the primer-nucleic acid template complexes under the action of the polymerase, thereby removing the second nucleotide.

Step (e22) involves contacting the third nucleotide, i.e., a nucleotide having a blocking group or an analog thereof, with the primer-nucleic acid template complexes under conditions suitable for a polymerization reaction, such that the third nucleotide is incorporated into the 3′ ends of the primer strands (including the first sequencing primer strand and the second sequencing primer strand) of the primer-nucleic acid template complexes. The type of the third nucleotide incorporated into the 3′ ends of the primer strands (including the first sequencing primer strand and the second sequencing primer strand) of the primer-nucleic acid template complexes is determined by the type of the second nucleotide determined by the foregoing step (d22), the two being theoretically in one to one correspondence.

In step (e22), the third nucleotide refers to a nucleotide or an analog thereof having, in the structure thereof, a blocking group capable of blocking the formation of a phosphate ester bond between the 3′-OH of the five-carbon sugar in the nucleotide or the analog thereof and a phosphate group of another nucleotide. The nucleotide or the analog thereof having a blocking group may not comprise a fluorescent label.

In step (e22), the polymerase may be a DNA polymerase or an RNA polymerase. In some examples, the polymerase is a DNA polymerase, including but not limited to one or more of 9° Nase or a mutant thereof, Pfu or a mutant thereof, KOD1 or a mutant thereof, Phi29 or a mutant thereof, Klenow or a mutant thereof, and MMS2 or a mutant thereof.

In some examples, a solution of the third nucleotide and the polymerase is contacted with the primer-nucleic acid template complexes, such that the third nucleotide is incorporated into the 3′ ends of the primer strands (including the first sequencing primer strand and the second sequencing primer strand) of the primer-nucleic acid template complexes under the action of the polymerase.

In some examples, a solution containing the third nucleotide and the polymerase is contacted with the primer-nucleic acid template complexes, such that the third nucleotide is incorporated into the 3′ ends of the primer strands (including the first sequencing primer strand and the second sequencing primer strand) of the primer-nucleic acid template complexes. The solution containing the third nucleotide and the polymerase may further contain a buffer, a divalent metal ion, an ammonium ion, etc., wherein the divalent metal ion may be at least one of Mg²⁺ and/or Mn²⁺, so as to promote the occurrence of the polymerization reaction.

In step (e22), the third nucleotide, the polymerase, and the primer-nucleic acid template complexes are contacted under conditions suitable for a polymerization reaction to occur, such that one nucleotide, the third nucleotide, having a blocking group is incorporated into the 3′ ends of the primer strands (including the first sequencing primer strand and the second sequencing primer strand) of at least one primer-nucleic acid template complex under the action of the polymerase. The conditions suitable for a polymerization reaction to occur refer to conditions capable of initiating a polymerization reaction between a phosphoric acid group of the nucleotide having a blocking group and the reactive groups at the 3′ ends of the primer strands. In some examples, the conditions suitable for a polymerization reaction to occur include one or two of the following conditions 1) and 2):
1) A reaction temperature of 30° C. to 75° C.; and
6) A pH of 8.0 to 10.

Illustratively, the foregoing reaction temperature may specifically be 30° C., 32° C., 35° C., 36° C., 37° C., 38° C., 39° C., 40° C., 41° C., 42° C., 43° C., 45° C., 46° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., etc.; the foregoing pH is specifically 8.0, 8.1, 8.2, 8.3, 8.4, 8.5, 8.6, 8.7, 8.7, 8.8, 8.9, 9.0, 9.1, 9.2, 9.3, 9.4, 9.5, 9.6, 9.7, 9.8, 9.8, 10.0, etc. Of course, different polymerization reaction conditions formed from any combination of the described reaction temperature and pH are also within the range of conditions suitable for a polymerization reaction to occur in examples of the present application.

The described reaction temperature and pH cover relatively mild reaction conditions, for example, a reaction temperature of 30° C. to 55° C., and a pH of 8.0 to 8.5. However, it is worth noting that the present application uses the foregoing sequencing primers to bind to the nucleic acid templates, which improves the stability of the primer-nucleic acid template complexes. However, it is worth noting that the present application uses the described sequencing primers to bind to the nucleic acid templates, which improves the stability of the primer-nucleic acid template complexes. In some examples, the conditions for the polymerization reaction include: a reaction condition of a pH of 9.1 to 10.0, or a reaction condition of a temperature of 60° C. to 75° C., or reaction conditions of a pH of 9.1 to 10.0 and a temperature of 60° C. to 75° C. In some examples, the conditions for the polymerization reaction include: a reaction condition of a pH of 9.8 to 10.0, or a reaction condition of a temperature of 65° C. to 75° C., or reaction conditions of a pH of 9.8 to 10.0 and a temperature of 65° C. to 75° C.

In some examples, the polymerase is Phi29 or Klenow or a mutant thereof, and the temperature suitable for the polymerization reaction to occur is 30° C. to 45° C. In some examples, the polymerase is 9° N or KOD or a mutant thereof, and the temperature suitable for the polymerization reaction to occur is 50° C. to 75° C.

In some examples, the step further comprises: washing to remove the third nucleotide which has not been reacted.

When step (b2) is (b23), step (c2) is (c23).

In some examples, after step (c23) or before step (f2), further comprised is: (d23) determining the base type of the third nucleotide of the first sequencing primer strand and of the second sequencing primer strand, respectively.

In one embodiment, (d23) comprises:
1) On the basis of the sequencing signals at one or multiple specified positions in the fluorescence image, determining a first sequencing signal component derived from the first sequencing primer strand and a second sequencing signal component derived from the second sequencing primer strand in the sequencing signals, respectively.

In this step, the specified position may be a region in the fluorescence image to which a position occupied by a cluster of nucleic acid templates on the solid phase substrate corresponds. The one or more specified positions at least include a first position, where fluorescent labels of the third nucleotides incorporated into the 3′ ends of the two sequencing primer strands are different, and the difference can be identified from the fluorescence image. That is, the sequencing signal at the first position includes two fluorescence signals of different colors. The two fluorescence signals are derived from the first sequencing primer extension strand and the second sequencing primer extension strand, respectively. Since, as described above, a portion of the first sequencing primer binding sites are blocked, in the two fluorescent signals, the signal with a larger sequencing signal component is classified as being derived from the second sequencing primer strand, and the signal with a smaller sequencing signal component is classified as being derived from the first sequencing primer strand. That is, the second sequencing signal component is greater than the first sequencing signal component.
2) Assigning the fluorescence signal corresponding to the first sequencing signal component in the fluorescence image obtained in step (c23) to the fluorescence signal of the first inserted nucleic acid fragment, and assigning the fluorescence signal corresponding to the second sequencing signal component to the fluorescence signal of the second inserted nucleic acid fragment.

For the fluorescence image obtained in step (c23) each time, the fluorescence signal corresponding to the first sequencing signal component in the fluorescence image obtained in step (c2) is assigned to the fluorescence signal of the first inserted nucleic acid fragment, and the fluorescence signal corresponding to the second sequencing signal component is assigned to the fluorescence signal of the second inserted nucleic acid fragment. The fluorescence signal is fluorescence color and fluorescence intensity. Thus, the type of the nucleotide reversible terminators incorporated into the first sequencing primer strand and the second sequencing primer strand in each round of polymerization reaction can be determined.
3) On the basis of the fluorescence signals to which the first sequencing signal component and the second sequencing signal component correspond, respectively, determining the base type of each nucleotide reversible terminator incorporated into the first sequencing primer strand and the second sequencing primer strand, respectively first inserted nucleic acid fragment.

In some examples, after step (d23), further comprised is step (e23): removing the fluorescently labeled antibody. In some examples, the fluorescently labeled antibody can be removed by heating, or the fluorescently labeled antibody bound to the extension product can be unbound by means of adding the third nucleotide and the polymerase.
(f2) Repeating step (b2) to step (c2) one or more times.

In this step, in each instance of step (b2) to step (c2), the extension of one nucleotide or one analog thereof at the 3′ ends of the primer strands is achieved, and the type of the nucleotide or the analog thereof incorporated into the primer strands is confirmed. By means of repeating multiple times, the determination of the sequence of the inserted nucleic acid fragments in the nucleic acid template sequence is achieved.

When the sequencing method comprises step (b21), step (f2) involves repeating step (b21) to step (d21) one or more times, so as to determine the sequence of the nucleic acid template. When the sequencing method comprises step (b22), step (f2) involves repeating step (b22) to step (e22) one or more times, so as to determine the sequence of the nucleic acid template. When the sequencing method comprises step (b23), step (f2) involves repeating step (b23) to step (e23) one or more times, so as to determine the sequence of the nucleic acid template.

The solutions of the present invention will be explained below in conjunction with examples. A person skilled in the art will understand that the following examples are only used to illustrate the present invention, and should not be construed as limiting the scope of the present invention. If no specific techniques or conditions are indicated in the examples, the techniques or conditions described in the literature in the art, or product specifications, are to be followed. The reagents or instruments used without indicating the manufacturers are all conventional products that can be obtained commercially.

### Example 1: Performing Sequencing Using Tripartite Primer P5-PEG-RD1

In this example, a tripartite primer P5-PEG-RD1 was constructed, which consisted of three of 5′-parts and had a structure AATGATACGGCGACCACCGAGATCTACAC/iSp18/ACACTCTTTCCCTACACGACGCTC TTCCGATCT-3 (SEQ ID NO: 1). At the same time, primer RD1 was constructed as a control. The structure of RD1 was 5′-ACACTCTTTCCCTACACGACGCTCTTCCGATCT-3′ (SEQ ID NO: 2).

Performing sequencing using tripartite primer P5-PEG-RD1 comprised:

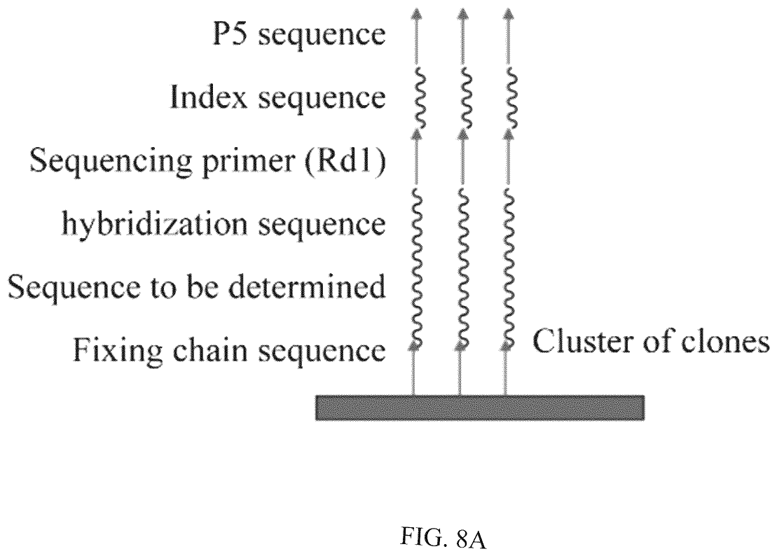

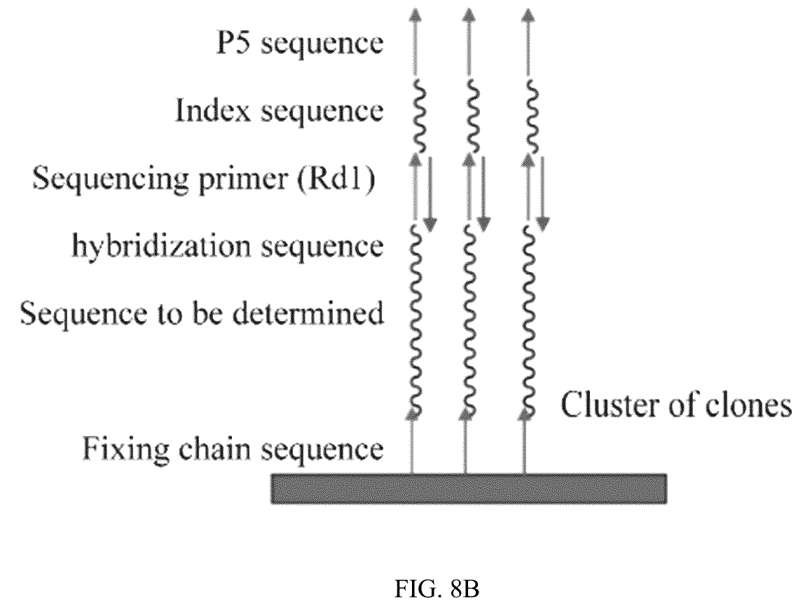

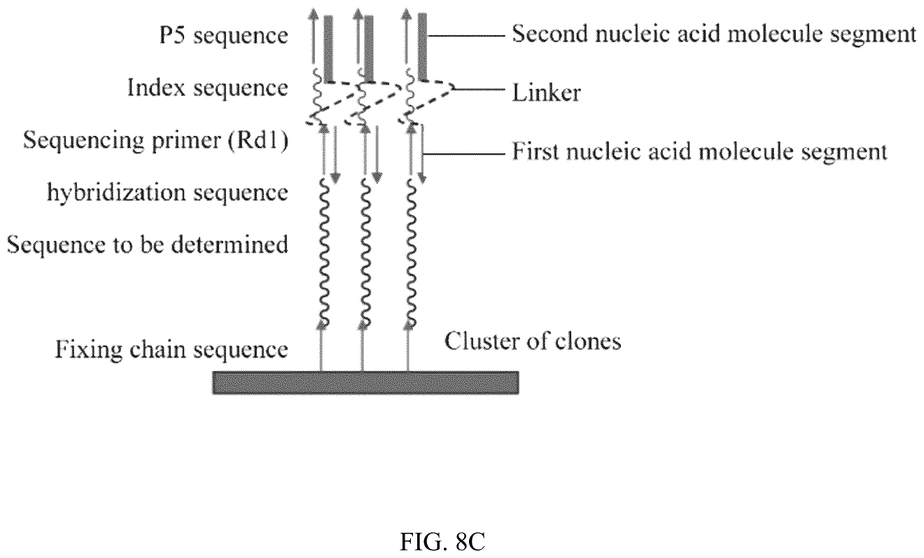

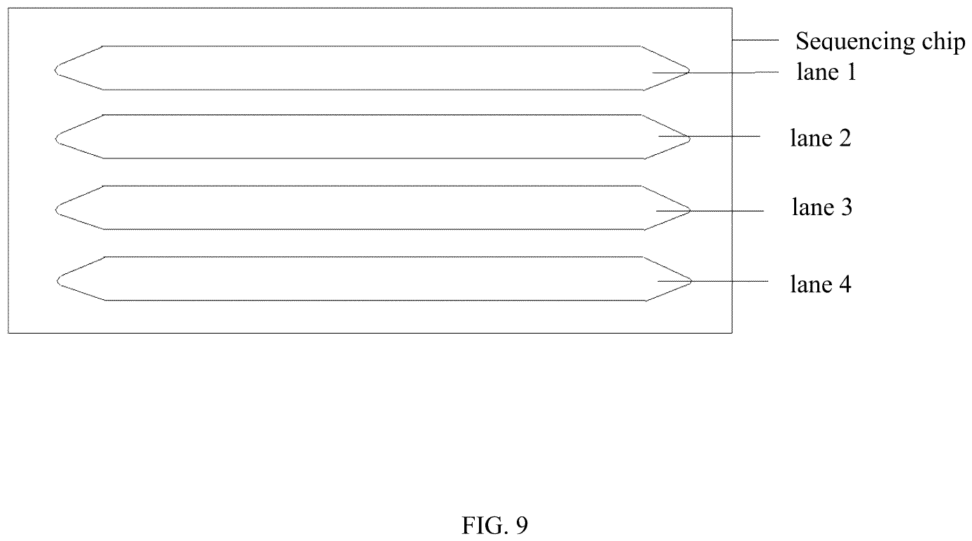

As shown in FIG. 8A, a cluster of clones of a nucleic acid template was fixed on a surface of a sequencing chip. The nucleic acid template comprised, from the 3′ end to the 5′ end, a P5 sequence, an Index sequence, a sequencing primer (Rd1) hybridization sequence, a sequence to be determined and a fixed strand sequence. As shown in FIG. 9, the sequencing chip was provided with four sequencing channels, which were lane1, lane2, lane3 and lane4, respectively.

As shown in FIG. 8C, under the condition of a temperature of 37° C., a buffer containing the tripartite primer P5-PEG-RD1 was introduced into lane2 and lane4 to allow hybridization with the nucleic acid template for 5 minutes to form a primer-nucleic acid template complex, wherein the primer concentration was 1 μmol/L, and the buffer was 5×SSC (Saline Sodium Citrate Buffer). At the same time, as shown in FIG. 8B, RD1 was used as a sequencing primer, and a buffer of RD1 was introduced into lane1 and lane3 under the same condition to allow hybridization with the nucleic acid template to form a primer-nucleic acid template complex as a control.

A synthetic reagent containing a nucleotide reversible terminator and a polymerase was added, and a polymerization reaction was performed at a temperature of 60° C. and at a pH of 9.80 and 10.0, respectively. The fluorescent signals on the surface of the sequencing chip after each round of polymerization reaction were collected. After the collection was completed, a reversible termination group of the nucleotide reversible terminator was removed, and a synthetic reagent containing the nucleotide reversible terminator and the polymerase was added again to perform a polymerization reaction. A total of 20 rounds of sequencing were completed.

Under the condition of pH 9.80, the intensities (in A.U.) of the fluorescence signals collected from the surface of the chip in the 1st round and the 10th round of sequencing are shown in Table 1 and Table 2 below, respectively.

TABLE 1

| | | | | | Average-base- |
|---|---|---|---|---|---|
| lane | Base A | Base C | Base G | Base T | 1st cycle |
| lane1 (control) | 153.227 | 102.85 | 158.967 | 50.101 | 116.28625 |
| lane2 | 148.892 | 95.93 | 151.522 | 48.484 | 111.207 |
| lane3 (control) | 146.667 | 105.256 | 161.447 | 51.978 | 116.337 |
| lane4 | 150.725 | 111.394 | 166.815 | 55.454 | 121.097 |

TABLE 2

| | | | | | | Strand |
|---|---|---|---|---|---|---|
| | | | | | Average-base- | detachment |
| lane | Base A | Base C | Base G | Base T | 10th cycle | percentage |
| lane1 (control) | 115.431 | 92.188 | 113.588 | 89.202 | 102.60225 | 11.8% |
| lane2 | 115.159 | 88.456 | 111.132 | 88.382 | 100.78225 | 9.4% |
| lane3 (control) | 117.051 | 91.976 | 118.484 | 91.237 | 104.687 | 10.0% |
| lane4 | 125.127 | 98.582 | 124.77 | 98.723 | 111.8005 | 7.7% |

As can be seen from Table 1 and Table 2, after 10 rounds of sequencing cycles using the tripartite primer P5-PEG-RD1 as the sequencing primer, the brightness decrease was more advantageous than that of the control examples using RD1 as the sequencing primer, indicating that the strand detachment percentage of the tripartite primer strand was lower than that of the RD1 primer strand. Specifically, the tripartite primer P5-PEG-RD1 was used as the sequencing primer to perform a polymerization reaction under the condition of pH 9.80, and the chain detachment percentage after 10 rounds of sequencing cycles was 7.7% and 9.4%, while the chain detachment percentage of the control primer was 10.0% and 11.8%. It can be seen that, under the condition of pH 9.8, using the tripartite primer P5-PEG-RD1 as the sequencing primer could improve the binding stability between the primer and the nucleic acid template.

Under the condition of pH 10.0, the intensities (in A.U.) of the fluorescence signals collected from the surface of the chip in the 1st round and the 10th round of sequencing are shown in Table 3 and Table 4 below, respectively.

TABLE 3

| | | | | | Average-base- |
|---|---|---|---|---|---|
| lane | Base A | Base C | Base G | Base T | 10th cycle |
| lane1 (control) | 154.116 | 107.902 | 163.429 | 53.735 | 119.7955 |
| lane2 | 154.881 | 105.144 | 161.778 | 54.766 | 119.14225 |
| lane3 (control) | 153.379 | 117.814 | 172.131 | 59.284 | 125.652 |
| lane4 | 157.742 | 120.31 | 173.98 | 61.732 | 128.441 |

TABLE 4

| | | | | | Average- | Strand |
|---|---|---|---|---|---|---|
| | | | | | base-1st | detachment |
| lane | Base A | Base C | Base G | Base T | cycle | percentage |
| lane1 (control) | 64.747 | 51.728 | 69.126 | 50.275 | 58.969 | 50.8% |
| lane2 | 114.536 | 93.09 | 116.542 | 88.968 | 103.284 | 13.3% |
| lane3 (control) | 71.389 | 57.169 | 78.735 | 56.322 | 65.90375 | 47.6% |
| lane4 | 127.031 | 106.831 | 133.477 | 101.747 | 117.2715 | 8.7% |

As can be seen from Table 1 and Table 2, after 10 rounds of sequencing cycle using the tripartite primer P5-PEG-RD1 as the sequencing primer, the brightness decrease was significantly more advantageous than that of the control examples using RD1 as the sequencing primer, and the strand detachment percentage of the tripartite primer strand was significantly lower than that of the RD1 primer strand. Specifically, the tripartite primer P5-PEG-RD1 was used as the sequencing primer to perform a polymerization reaction under the condition of a pH of 10.0, and the chain detachment percentage after 10 rounds of sequencing cycle was 8.7% and 13.3%, while the chain detachment percentage of the control primer was as high as 47.6% and 50.8%. It can be seen that, under the condition of pH 10.0, using the tripartite primer P5-PEG-RD1 as the sequencing primer could improve the binding stability between the primer and the nucleic acid template.

In addition, compared with the polymerization reaction condition of pH 9.8, under the polymerization reaction condition of pH 10.0, the strand detachment percentage after 10 rounds of sequencing was significantly increased in the control examples; whereas in the experimental examples using the tripartite primer P5-PEG-RD1 as the sequencing primer, the decrease in strand detachment percentage was slow as the pH increased. This indicates that using the tripartite primer P5-PEG-RD1 to improve the binding stability between the primer and a nucleic acid template can allow a sequencing reaction to be performed under harsher reaction conditions, and can afford a better sequencing quality.

### Example 2: Using Tripartite Primer sR Int to Perform Synchronous Sequencing on a Fragment with Two Inserts

In this example, four primers were constructed as shown in Table 5 below.

TABLE 5

| | | 3′ |
|---|---|---|
| Primer | Sequence | hydroxyl |
| RD2 | 5′-ACACGACGCTCTTCCGATCT-3′ | Unblocked |
| | (SEQ ID NO: 3) | |
| sR | 5′-CACGTTCAGAGTTCTACAGTCCG | Unblocked |
| | ACGATC-3′ | |
| | (SEQ ID NO: 4) | |
| sR-3Am | 5′-CACGTTCAGAGTTCTACAGTCCG | 3 Am |
| | ACGATC-3′ | blocked |
| | (SEQ ID NO: 5) | |
| Tripartite | 5′-CACGTTCAGAGTTCTACAGTCCG | Unblocked |
| primer | ACGATC/isp18/CACGTTCAGAGTT | |
| sR int | CTACAGTCCGACGATC-3′ | |
| | (SEQ ID NO: 6) | |

The sequencing comprised:

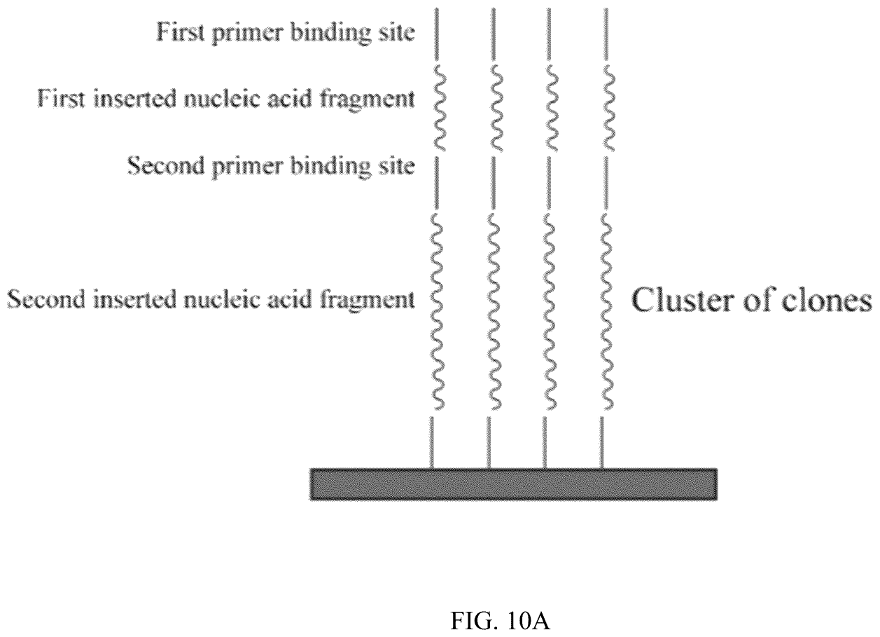

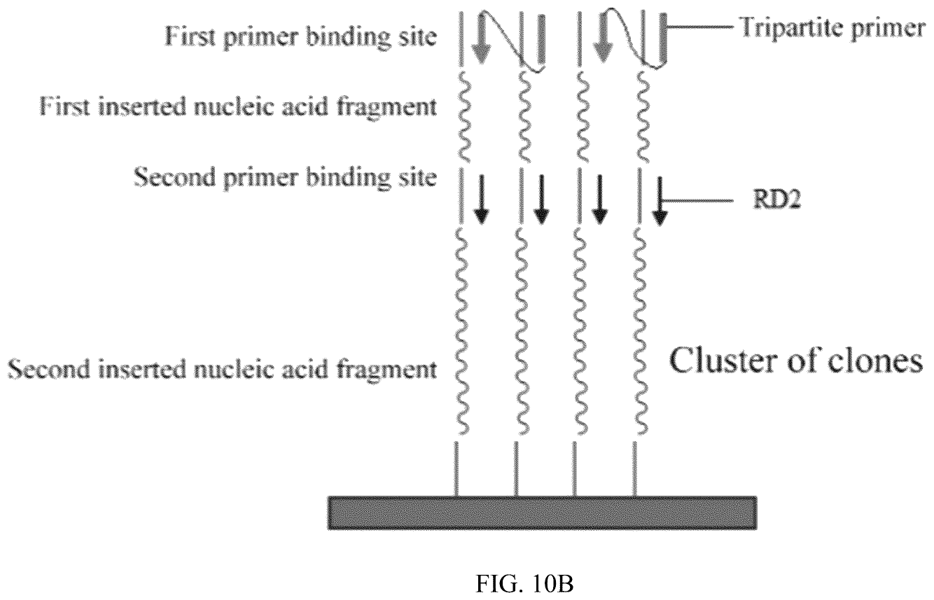

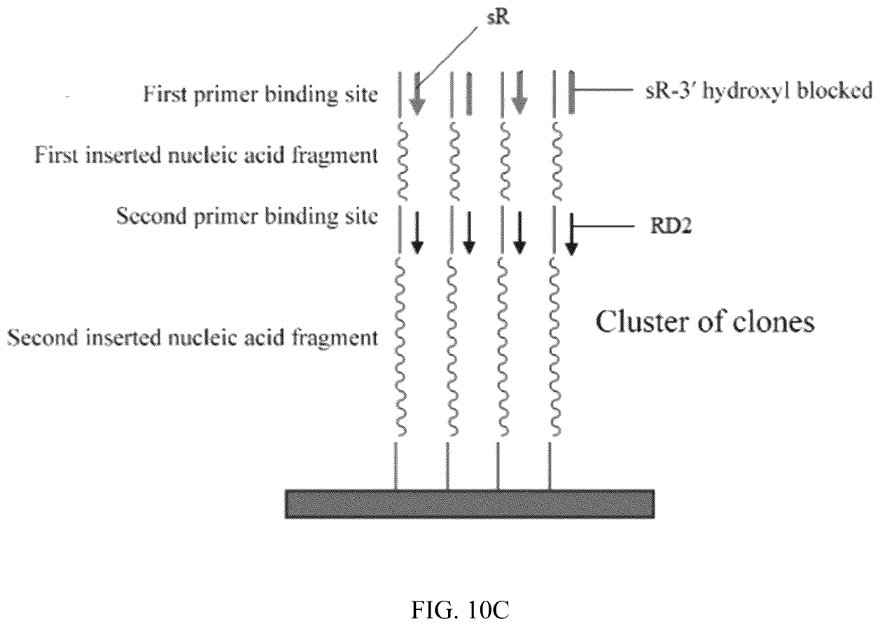

As shown in FIG. 10A, a cluster of clones of a nucleic acid template was fixed on a surface of a sequencing chip. The nucleic acid template comprised, from the 3′ end to the 5′ end, a first primer binding site, a first inserted nucleic acid fragment (or a first sequence to be determined), a second primer binding site, a second inserted nucleic acid fragment (or a second sequence to be determined), and a fixed strand sequence. The first primer binding site is complementary to a 3′ end sequence of the tripartite primer sR-int, a 5′ end sequence of sR-int, the sR sequence, the sR-3ddC sequence and the sR-3Am sequence, and the second primer binding site is complementary to the RD2 sequence.

As shown in FIG. 10B, under the condition of a temperature of 37° C., a buffer containing the sR-int primer (tripartite primer) and the RD2 primer was introduced into a surface of a sequencing chip to allow hybridization with the nucleic acid template for 5 minutes to form a primer-nucleic acid template complex, wherein the primer concentration was 1 μmol/L, and the buffer was 5×SSC (Saline Sodium Citrate Buffer). At the same time, as shown in FIG. 10C, a mixed primer of sR and sR-3Am in a molar ratio of 1:1 and RD2 was used as a sequencing primer to hybridize with the nucleic acid template under the same conditions to form a primer-nucleic acid template complex as a control.

A synthetic reagent containing a nucleotide reversible terminator and a polymerase was added, and a polymerization reaction was performed at a temperature of 60° C. and at a pH of 9.0. The fluorescent signals on the surface of the sequencing chip after each round of polymerization reaction were collected. After the collection was completed, a reversible termination group of the nucleotide reversible terminator was removed, and a synthetic reagent containing the nucleotide reversible terminator and the polymerase was added again to perform a polymerization reaction.

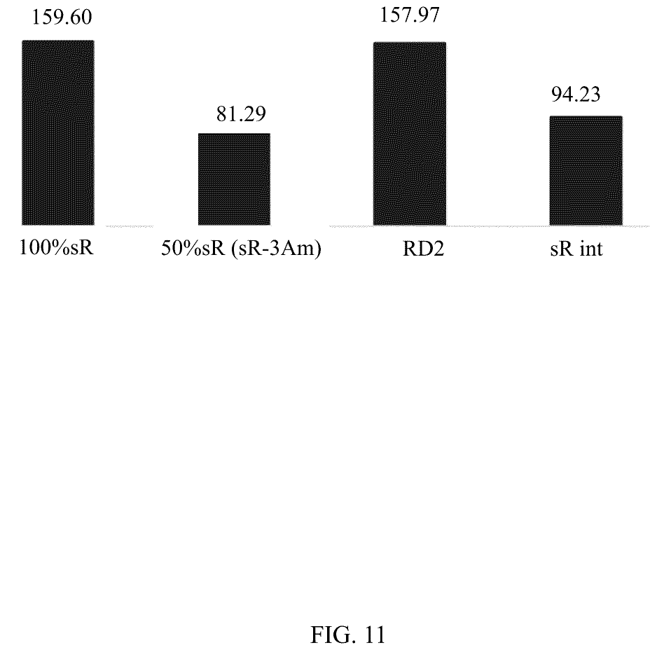

The intensities of the fluorescent signals collected from the surface of the chip in the 11th to 20th rounds of sequencing were summed, and the average value was calculated. The results are as shown in FIG. 11, and it can be seen from the figure that: compared with the signal intensity when the RD2 primer was used alone for sequencing, when the tripartite primer (sR Int) was used as the sequencing primer, the decrease in signal intensity was less than half, indicating that using the tripartite primer could improve the binding stability between the primer and the target nucleic acid molecule and reduce the strand detachment proportion. In addition, compared with the sequencing on a complex obtained by using 50% blocking primer (50% sR-3Am) for hybridization, the signal intensity obtained when using the tripartite primer (sR Int) as the sequencing primer was slightly higher, but the fluctuation in brightness of the tripartite primer (sR Int) example was smaller. The smaller the fluctuation in brightness, the easier it is to identify and distinguish the two fluorescence signals.

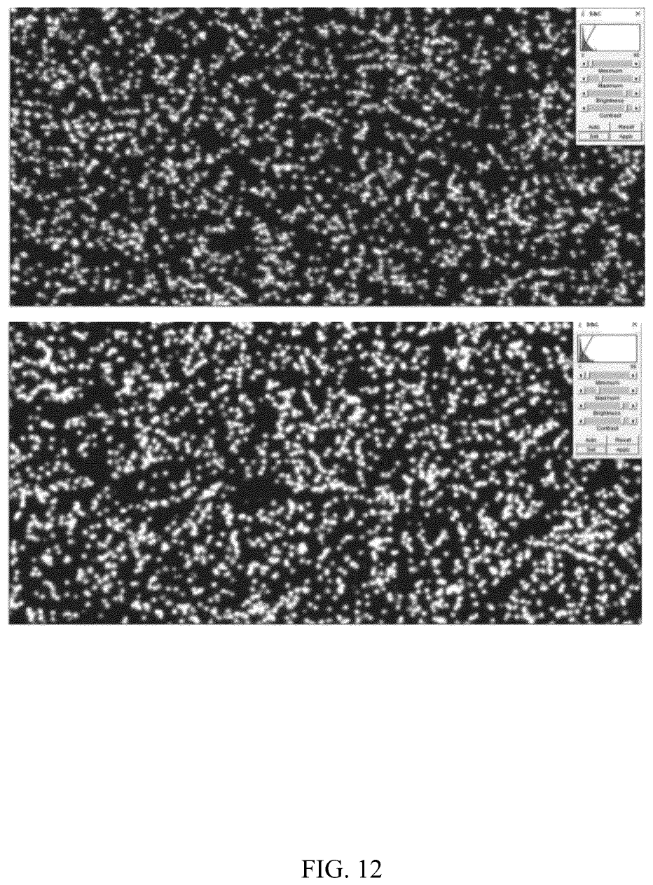

The tripartite primer of the Example and the control group were each used to perform 200 rounds of sequencing under the same conditions, and the fluorescence signals determined from one fluorescence signal channel in one of the sequencing rounds were collected for analysis. FIG. 12 shows fluorescence images of base G obtained in the 5th round of sequencing, wherein the upper panel shows the fluorescence image of base G of the control group, and the lower panel shows the fluorescence image of base G of the Example using the tripartite primer to perform sequencing. FIG. 12 shows that: in a center region of the same gray scale, the brightness of the image of base G when using the mixed primer of sR:sR 3Am in a molar ratio of 1:1 to perform sequencing was weaker than that of the image of base G when using primer sR int to perform sequencing. The average value, variance and CV of the brightness of the signals of base G in the two panels of FIG. 12 were calculated, as shown in Table 6 below. It can be seen that the average value of the fluorescence intensities of the fluorescence signals from sequencing using the tripartite primer of the Example was similar to that of the control example, but the variance and CV were significantly lower, facilitating base identification.

TABLE 6

| Item | Control group | Example |
|---|---|---|
| Mean (average value) | 241.93 | 239.75 |
| Stdev (variance) | 13.99 | 8.61 |
| CV | 5.78% | 3.55% |

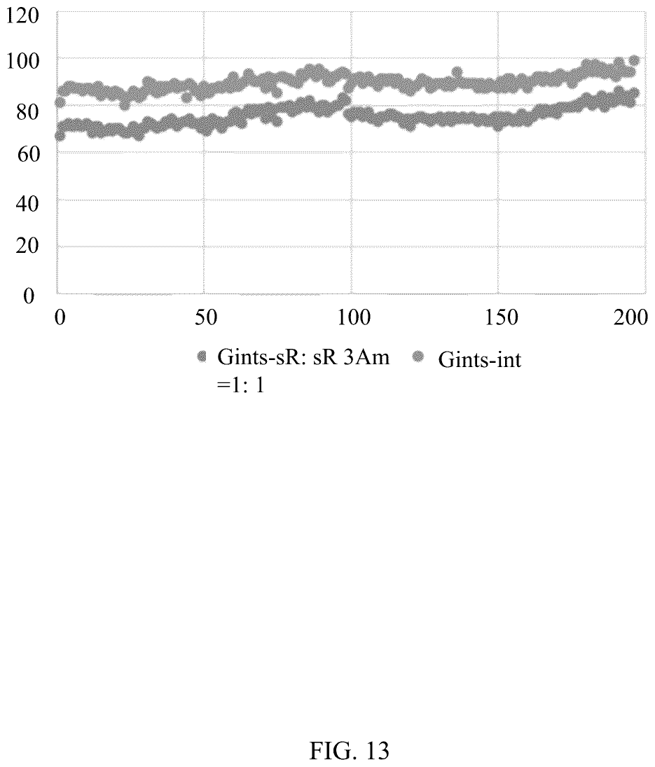

The tripartite primer of the Example and the control group were each used to perform 200 rounds of sequencing under the same conditions. The fluorescence signal intensities collected are as shown in FIG. 13, in which the upper curve represents the change in fluorescence signal intensity of the control group in 200 rounds of sequencing, and the lower curve represents the change in fluorescence signal intensity of the Example, which used the tripartite primer to perform sequencing, in 200 rounds of sequencing. The mean CV value of the Example and the control group were calculated to be 3.57% and 5.3%, respectively. It can be seen from the figure that the Example which used the tripartite primer to perform sequencing had less fluctuation in sequencing signal and lower CV value of signal intensity in 200 rounds of sequencing, which helps to distinguish and identify the sequencing signals of two inserted nucleic acid fragments by using brightness information, and can improve the accuracy.

In the description of the present specification, description with reference to terms such as “one example”, “some example”, “exemplification”, “specific exemplification”, or “some exemplifications”, etc. denotes that a specific feature, structure, material or characteristic described in conjunction with the example or exemplification is included in at least one example or exemplification of the present invention. In the present specification, the schematic expressions of the described terms do not necessarily refer to the same example or exemplification. Moreover, the specific feature, structure, material or characteristic described may be combined in any one or more examples or exemplifications in a suitable manner.

Although the examples of the present invention have been illustrated and described, it may be appreciated that the described examples are illustrative, and should not be construed as a limitation to the present invention. A person of ordinary skilled in the art can make changes, modifications, substitutions and variations to the foregoing examples within the scope of the present invention without departing from the principle and spirit of the present invention.

What is claimed is:

1. A sequencing primer, characterized by comprising:
a first nucleic acid segment, the first nucleic acid segment having a reactive group at the 3′ end, the reactive group having reactivity to undergo a polymerization reaction with a nucleotide or an analog thereof, and the first nucleic acid segment being configured to specifically bind to at least a portion of a sequence within a first region of a target nucleic acid molecule;
a second nucleic acid segment, the second nucleic acid segment being configured to specifically bind to at least a portion of a sequence within a second region of a target nucleic acid molecule; and
a linker, the linker being bound to the 5′ end of the first nucleic acid segment and the 3′ end of the second nucleic acid segment, respectively, or the linker being bound to the 5′ end of the first nucleic acid segment and the 5′ end of the second nucleic acid segment, respectively, and when the linker is bound to the 5′ end of the first nucleic acid segment, the first nucleic acid segment having a 3′ occluded end.

2. The sequencing primer according to claim 1, wherein the reactive group comprises —OH.

3. The sequencing primer according to claim 1, wherein the linker is bound to the 5′ end of the first nucleic acid segment through a first linking group, and the linker is bound to the 3′ end or 5′ end of the second nucleic acid segment through a second linking group.

4. The sequencing primer according to claim 3, wherein the first linking group and/or the second linking group are each independently selected from a hydroxyl group, a carboxyl group, a sulfonate group, an ester group, a phosphate ester group, an amino group, an ether group, a saccharide group, an alkyl group, an amide group, an aromatic ring, or a biological macromolecule having a molecular weight of no more than 100,000.

5. The sequencing primer according to claim 4, wherein the biological macromolecule is selected from a polysaccharide, a polypeptide chain, and an oligomeric nucleic acid, and the linker has a length of 0.3 nm to 100 nm.

6. The sequencing primer according to claim 4, wherein the linker is selected from a polymer, and the polymer comprises at least one selected from polyethylene glycol, polyvinyl alcohol, polyacrylamide, polymethacrylic acid, polyvinyl sulfonic acid, and polyvinylpyrrolidone.

7. The sequencing primer according to claim 4, wherein the linker is selected from a nucleotide or polynucleotide composed of 1 to 40 bases, preferably a polynucleotide composed of 3 to 26 bases.

8. The sequencing primer according to claim 4, wherein the linker is selected from a small molecule compound the number of backbone atoms in which is greater than or equal to 6 and a molecular weight of which is no greater than 1,000.

9. The sequencing primer according to claim 1, wherein in each sequencing primer, the molar ratio of the first nucleic acid segment to the second nucleic acid segment is 1:10 to 10:1.

10. The sequencing primer according to claim 1, wherein the target nucleic acid molecule comprises an inserted nucleic acid fragment, and a first region and a second region located at the 3′ end of the inserted nucleic acid fragment, the first segment nucleic acid molecule is configured to specifically bind to the first region of the sequencing library molecule, and the second nucleic acid segment is configured to specifically bind to the second region of the sequencing library molecule.

11. The sequencing primer according to claim 10, wherein a spacer sequence is provided between the first region and the second region, and the length of the linker is greater than the length of the spacer sequence.

12. The sequencing primer according to claim 1, wherein there are multiple target nucleic acid molecules, and the multiple target nucleic acid molecules are derived from the same cluster of nucleic acid molecule clones, the target nucleic acid molecules each comprising a first inserted nucleic acid fragment and a second inserted nucleic acid fragment, and the second inserted nucleic acid fragment being located to the 5′ end of the first inserted nucleic acid fragment; the 3′ end of the first inserted nucleic acid fragment is provided with a first primer binding site, and the first region and the second region are the first primer binding sites of different target nucleic acid molecules.

13. A sequencing method, characterized by comprising:
(a1) hybridizing a sequencing primer to a nucleic acid template, to form a primer-nucleic acid template complex, wherein the nucleic acid template comprises an inserted nucleic acid fragment and, at the 3′ end of the inserted fragment, a sequencing primer binding site and a known sequence, the known sequence being located at the 3′ end of the sequencing primer binding site, and a spacer sequence being provided between the sequencing primer binding site and the known sequence; the sequencing primer comprises a linker, a first nucleic acid segment, and a second nucleic acid segment, two ends of the linker binding to the 5′ end of the first nucleic acid segment and the 3′ end of the second nucleic acid segment, respectively, and the 3′ end of the first nucleic acid segment having a reactive group, the reactive group having reactivity to undergo a polymerization reaction with a nucleotide or an analog thereof; in the primer-nucleic acid template complex, the first nucleic acid segment is partially complementary or completely complementary to the sequencing primer binding site, the second nucleic acid segment is partially complementary or completely complementary to the known sequence of the nucleic acid template, and the sequence of the first nucleic acid segment is different from the sequence of the second nucleic acid segment;
(b1) contacting one of the following substrates or substrate combinations i) to iii) and a polymerase with the primer-nucleic acid template complex, such that the substrate or substrate combination binds to the polymerase and the primer-nucleic acid template complex, and/or the substrate or substrate combination is incorporated into the 3′ end of the primer strand of the primer-nucleic acid template complex,
i) a first nucleotide, the first nucleotide comprising a fluorescent label, and a blocking group used to prevent the incorporation of a next nucleotide or an analog thereof into the primer strand,
ii) a second nucleotide, the second nucleotide being a nucleotide containing a fluorescent label, or an analog thereof, and
iii) a third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand;
(c1) exciting the fluorescent label to generate a fluorescence signal, and collecting a fluorescence image; and
(e1) repeating steps (b1) to step (c1) one or more times.

14. The sequencing method according to claim 13, wherein, in step (b1), the substrate is i), and the first nucleotide, the polymerase, and the primer-nucleic acid template complex are contacted under conditions suitable for a polymerization reaction to occur, such that the first nucleotide is incorporated into the 3′ end of the primer strand of the primer-nucleic acid template complex;
step (c1) further comprises: determining, based on the fluorescence image, the type of the nucleotide or the analog incorporated into the primer strand.

15. The sequencing method according to claim 13, wherein, in step (b1), the substrate is ii), and the second nucleotide, the polymerase and the primer-nucleic acid template complex are contacted under non-polymerization reaction conditions, such that the second nucleotide binds to the polymerase and the primer-nucleic acid template complex;
step (c1) further comprises: determining, based on the fluorescence image, the type of the nucleotide or the analog thereof bound to the polymerase;
after step (c1), further comprised is:
(d1) removing the second nucleotide; contacting the third nucleotide, the polymerase, and the primer-nucleic acid template complex under conditions suitable for a polymerization reaction, so as to incorporate the third nucleotide into the 3′ end of the primer strand of the primer-nucleic acid template;
contacting under non-polymerization reaction conditions comprises:
contacting under conditions in which Mg²⁺ and/or Mn²⁺ are not contained,
contacting under conditions in which at least one of Ca²⁺, Ni²⁺, and Si²⁺ is contained.

16. The sequencing method according to claim 13, wherein, in step (b1), the substrate is iii), and the third nucleotide, the fluorescently labeled antibody, the polymerase, and the primer-nucleic acid template complex are contacted under conditions suitable for a polymerization reaction, such that a nucleotide or an analog thereof having a blocking group is incorporated into the 3′ end of the primer strand of the primer-nucleic acid template complex, and the fluorescently labeled antibody binds to the third nucleotide;
step (c1) further comprises: determining, based on the fluorescence image, the type of the nucleotide or the analog thereof bound to the polymerase;
after step (c1), further comprised is:
(d1) removing the fluorescently labeled antibody.

17. A sequencing method, characterized by comprising:
(a2) hybridizing a first sequencing primer and a second sequencing primer to a cluster of nucleic acid templates, to form primer-nucleic acid template complexes, wherein the cluster of nucleic acid templates at least comprises a first nucleic acid template and a second nucleic acid template, the first nucleic acid template and the second nucleic acid template each comprise a first inserted nucleic acid fragment and a second inserted nucleic acid fragment, and a first primer binding site and a second primer binding site located at the 3′ end of the first inserted nucleic acid fragment and the 3′ end of the second inserted nucleic acid fragment, respectively, and the first inserted nucleic acid fragment is located at the 3′ end of the second inserted nucleic acid fragment; the first sequencing primer comprises a linker, a first nucleic acid segment and a second nucleic acid segment, two ends of the linker are bound to the 5′ end of the first nucleic acid segment and the 3′ end or 5′ end of the second nucleic acid segment, respectively, and the 3′ end of the first nucleic acid segment has a reactive group, the reactive group having reactivity to undergo a polymerization reaction with a nucleotide or an analog thereof, and when the linker binds to the 5′ end of the second nucleic acid segment, the second nucleic acid segment has a 3′ occluded end; in the primer-nucleic acid template complexes, the first nucleic acid segment is partially complementary or completely complementary to the first primer binding site of the first nucleic acid template, the second nucleic acid segment is partially complementary or completely complementary to the first primer binding site of the second nucleic acid template, the second sequencing primer is partially complementary or completely complementary to the second primer binding site of the nucleic acid templates, and the sequence of the first nucleic acid segment is identical or complementary to the sequence of the second nucleic acid segment; the 3′ end of the second sequencing primer has a reactive group;
(b2) contacting one of the following substrates or substrate combinations i) to iii) and a polymerase with the primer-nucleic acid template complexes, such that the substrate or substrate combination is bound to the polymerase and the primer-nucleic acid template complexes, and/or the substrate or substrate combination is incorporated into the 3′ end of the primer strand of the primer-nucleic acid template complexes,
i) a first nucleotide, the first nucleotide comprising a fluorescent label, and a blocking group used to prevent the incorporation of a next nucleotide or an analog thereof into the primer strand,
ii) a second nucleotide, the second nucleotide being a nucleotide containing a fluorescent label, or an analog thereof, and
iii) a third nucleotide and a fluorescently labeled antibody, wherein the third nucleotide is a fluorescent label-free nucleotide or an analog thereof, and the third nucleotide comprises a blocking group used to prevent the incorporation of a next nucleotide or an analog thereof into the primer strand;
(c2) exciting the fluorescent label to generate a fluorescence signal, and collecting a fluorescence image; and
(f2) repeating steps (b2) to step (c2) one or more times.

18. The sequencing method according to claim 17, wherein, in step (b2), the fluorescently labeled antibody substrate is i), and the first nucleotide, the polymerase and the primer-nucleic acid template complexes are contacted under conditions suitable for a polymerization reaction to occur, such that the first nucleotide is incorporated into the 3′ end of the primer strand of the primer-nucleic acid template complexes;
step (c2) further comprises: determining, based on the fluorescence image, the type of the nucleotide or the analog incorporated into the primer strand.

19. The sequencing method according to claim 17, wherein, in step (b2), the substrate is ii), and the second nucleotide, the polymerase and the primer-nucleic acid template complexes are contacted under non-polymerization reaction conditions, such that the second nucleotide binds to the polymerase and the primer-nucleic acid template complexes;
step (c2) further comprises: determining, based on the fluorescence image, the type of the nucleotide or the analog thereof bound to the polymerase;
after step (c2), further comprised is:
(d2) removing the second nucleotide; contacting the third nucleotide, the polymerase and the primer-nucleic acid template complex under conditions suitable for a polymerization reaction, so as to incorporate the third nucleotide into the 3′ end of the primer strand of the primer-nucleic acid template;
contacting under non-polymerization reaction conditions comprises:
contacting under conditions in which Mg²⁺ and/or Mn²⁺ are not contained,
contacting under conditions in which at least one of Ca²⁺, Ni²⁺ and Si²⁺ is contained.

20. The sequencing method according to claim 17, wherein, in step (b2), the substrate is iii), and the third nucleotide, the fluorescently labeled antibody, the polymerase and the primer-nucleic acid template complex are contacted under conditions suitable for a polymerization reaction, such that a nucleotide or an analog thereof having a blocking group is incorporated into the 3′ end of the primer strand of the primer-nucleic acid template complex, and the fluorescently labeled antibody binds to the third nucleotide;
step (c2) further comprises: determining, based on the fluorescence image, the type of the nucleotide or the analog thereof bound to the polymerase;
after step (c2), further comprised is:
(d2) removing the fluorescently labeled antibody.


SEQUENCE LISTING (6 sequences)

SEQ ID NO: 1 - DNA, 62 bp, synthetic construct
  source 1..62: mol_type = other DNA
aatgatacggcgaccaccgagatctacacacactctttccctacacgacgctcttccgatct

SEQ ID NO: 2 - DNA, 33 bp, synthetic construct
  source 1..33: mol_type = other DNA
acactctttccctacacgacgctcttccgatct

SEQ ID NO: 3 - DNA, 20 bp, synthetic construct
  source 1..20: mol_type = other DNA
acacgacgctcttccgatct

SEQ ID NO: 4 - DNA, 29 bp, synthetic construct
  source 1..29: mol_type = other DNA
cacgttcagagttctacagtccgacgatc

SEQ ID NO: 5 - DNA, 29 bp, synthetic construct
  source 1..29: mol_type = other DNA
cacgttcagagttctacagtccgacgatc

SEQ ID NO: 6 - DNA, 58 bp, synthetic construct
  source 1..58: mol_type = other DNA
cacgttcagagttctacagtccgacgatccacgttcagagttctacagtccgacgatc